(19) United States
(12) Patent Application Publication
(10) Pub. No.: US 2009/0076081 A1
(43) Pub. Date: Mar. 19, 2009

(54) PIPERIDINE DERIVATIVES AS NK3 ANTAGONISTS

(76) Inventors: Henner Knust, Rheinfelden (DE); Matthias Nettekoven, Grenzach-Wyhlen (DE); Hasane Ratni, Habsheim (FR); Walter Vifian, Olten (CH); Xihan Wu, Shanghai (CN)

Correspondence Address: HOFFMANN-LA ROCHE INC.;PATENT LAW DEPARTMENT, 340 KINGSLAND STREET, NUTLEY, NJ, 07110, US

(21) Appl. No.: 12/207,596

(22) Filed: Sep. 10, 2008

(30) Foreign Application Priority Data
Sep. 14, 2007 (EP) 07116441.2

(51) Int. Cl. A61K 31/445; C07D 211/22; A61K 31/4523; A61P 25/24; A61P 25/16; C07D 401/04

(52) U.S. Cl. 514/326; 546/237; 546/208; 514/331

(57) ABSTRACT

The invention relates to a compound of formula

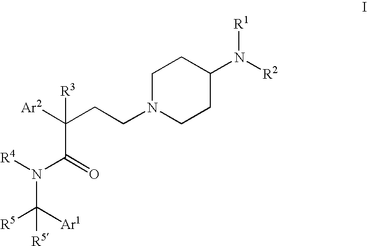

wherein
Ar¹, Ar², R¹, R², R³, R⁴, R⁵, and R^{5′} or to a pharmaceutically active salt thereof.

The compounds are high potential NK-3 receptor antagonists for the treatment of depression, pain, psychosis, Parkinson's disease, schizophrenia, anxiety and attention deficit hyperactivity disorder (ADHD).

### PRIORITY TO RELATED APPLICATION(S)

This application claims the benefit of European Patent Application No. 07116441.2, filed Sep. 14, 2007, which is hereby incorporated by reference in its entirety.

### BACKGROUND OF THE INVENTION

The three main mammalian tachykinins, substance P (SP), neurokinin A (NKA) and neurokinin B (NKB) belong to the family of neuropeptides sharing the common COOH-terminal pentapeptide sequence of Phe-X-Gly-Leu-Met-NH₂. As neurotransmitters, these peptides exert their biological activity via three distinct neurokinin (NK) receptors termed as NK-1, NK-2 and NK-3. SP binds preferentially to the NK-1 receptor, NKA to the NK-2 and NKB to the NK-3 receptor.

The NK-3 receptor is characterized by a predominant expression in CNS and its involvement in the modulation of the central monoaminergic system has been shown. These properties make the NK-3 receptor a potential target for central nervous system disorders such as anxiety, depression, bipolar disorders, Parkinson's disease, schizophrenia and pain (Neurosci. Letters, 2000, 283, 185-188; Exp. Opin. Ther. Patents 2000, 10, 939-960; Neuroscience, 1996, 74, 403-414; Neuropeptides, 1998, 32, 481-488).

Schizophrenia is one of the major neuropsychiatric disorders, characterized by severe and chronic mental impairment. This devastating disease affects about 1% of the world's population. Symptoms begin in early adulthood and are followed by a period of interpersonal and social dysfunction. Schizophrenia manifests as auditory and visual hallucinations, paranoia, delusions (positive symptoms), blunted affect, depression, anhedonia, poverty of speech, memory and attention deficits as well as social withdrawal (negative symptoms).

For decades scientists and clinicians have made efforts with the aim of discovering an ideal agent for the pharmacological treatment of schizophrenia. However, the complexity of the disorders, due to a wide array of symptoms, has hampered those efforts. There are no specific focal characteristics for the diagnosis of schizophrenia and no single symptom is consistently present in all patients. Consequently, the diagnosis of schizophrenia as a single disorder or as a variety of different disorders has been discussed but not yet resolved. The major difficulty in the development of a new drug for schizophrenia is the lack of knowledge about the cause and nature of this disease. Some neurochemical hypotheses have been proposed on the basis of pharmacological studies to rationalize the development of a corresponding therapy: the dopamine, the serotonin and the glutamate hypotheses. But taking into account the complexity of schizophrenia, an appropriate multireceptor affinity profile might be required for efficacy against positive and negative signs and symptoms. Furthermore, an ideal drug against schizophrenia would preferably have a low dosage allowing once-per-day dosage, due to the low adherence of schizophrenic patients.

In recent years clinical studies with selective NK1 and NK2 receptor antagonists appeared in the literature showing results for the treatment of emesis, depression, anxiety, pain and migraine (NK1) and asthma (NK2 and NK1). The most exciting data were produced in the treatment of chemotherapy-induced emesis, nausea and depression with NK1 and in asthma with NK2-receptor antagonists. In contrast, no clinical data on NK3 receptor antagonists have appeared in the literature until 2000. Osanetant (SR 142,801) from Sanofi-Synthelabo was the first identified potent and selective non-peptide antagonist described for the NK3 tachykinin receptor for the potential treatment of schizophrenia, which was reported in the literature (Current Opinion in Investigational Drugs, 2001, 2(7), 950-956 and Psychiatric Disorders Study 4, Schizophrenia, June 2003, Decision Recources, Inc., Waltham, Mass.). The proposed drug SR 142,801 has been shown in a phase II trial as active on positive symptoms of schizophrenia, such as altered behaviour, delusion, hallucinations, extreme emotions, excited motor activity and incoherent speech, but inactive in the treatment of negative symptoms, which are depression, anhedonia, social isolation or memory and attention deficits.

The neurokinin-3 receptor antagonists have been described as useful in pain or inflammation, as well as in schizophrenia, Exp. Opinion. Ther. Patents (2000), 10(6), 939-960 and Current Opinion in Investigational Drugs, 2001, 2(7), 950-956 956 and Psychiatric Disorders Study 4, Schizophrenia, June 2003, Decision Recources, Inc., Waltham, Mass.).

### SUMMARY OF THE INVENTION

The invention provides compounds of formula I

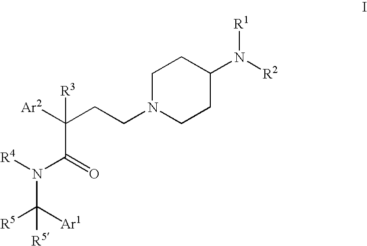

wherein
Ar¹ and Ar² are each independently phenyl or pyridinyl, each of which is optionally substituted by one or two substituents selected from the group consisting of halogen, lower alkyl, lower alkoxy, lower alkyl substituted by halogen, lower alkoxy substituted by halogen, lower alkyl substituted by alkoxy, lower alkyl substituted by cyano, lower di-alkyl amino, pyridinyl and cyano;
R¹ is hydrogen, lower alkyl, —(CH₂)₂O-lower alkyl or cycloalkyl:
R² is —S(O)₂-lower alkyl or —C(O)-lower alkyl;
or R¹ and R² together with the N-atom to which they are attached form a pyrrolidin-2-one or a piperidin-2-one group;
R³ is hydrogen, halogen or lower alkyl;
R⁴ is hydrogen or lower alkyl;
R⁵ and R^{5′} are each independently hydrogen, lower alkyl, lower alkyl substituted by halogen, lower alkyl substituted by hydroxy or cycloalkyl;
or R⁵ and R⁵ together with the carbon-atom to which they are attached form a cycloalkyl group;
and pharmaceutically active salts thereof.

The invention includes all stereoisomeric forms, including individual diastereoisomers and enantiomers of the compound of formula (I) as well as racemic and non-racemic mixtures thereof.

The present invention also provides pharmaceutical compositions containing one or more compounds of formula I. The invention further provides processes for the preparation of the compounds and compositions of the invention.

The present compounds are high potential NK-3 receptor antagonists for the treatment of depression, pain, biopolar disorders, psychosis, Parkinson's disease, schizophrenia, anxiety and attention deficit hyperactivity disorder (ADHD).

The preferred indications using the compounds of the present invention are depression, psychosis, Parkinson's disease, schizophrenia, anxiety and attention deficit hyperactivity disorder (ADHD).

### DETAILED DESCRIPTION OF THE INVENTION

The following definitions of the general terms used in the present description apply irrespective of whether the terms in question appear alone or in combination.

As used herein, the term “lower alkyl” denotes a straight- or branched-chain hydrocarbon group containing from 1-8 carbon atoms, for example, methyl, ethyl, propyl, isopropyl, n-butyl, i-butyl, t-butyl and the like. Preferred lower alkyl groups are groups with 1-4 carbon atoms.

The term “halogen” denotes chlorine, iodine, fluorine and bromine.

The term “lower alkyl substituted by halogen” denotes an alkyl group as defined above, wherein at least one hydrogen atom is replaced by halogen, for example —CF₃, —CHF₂, —CH₂F, —CH₂CF₃, —CH₂CH₂CF₃, —CH₂CF₂CF₃ and the like. Preferred lower alkyl substituted by halo groups are groups having 1-4 carbon atoms.

The term “lower alkyl substituted by hydroxy” denotes an alkyl group as defined above, wherein at least one hydrogen atom is replaced by a hydroxy, for example —CH₂OH, —CH₂CH₂OH, and the like.

The term “lower alkyl substituted by CN” denotes an alkyl group as defined above, wherein at least one hydrogen atom is replaced by a cyano group, for example —CH₂CN, —CH₂CH₂CN, and the like.

The term “lower alkyl substituted by alkoxy” denotes an alkyl group as defined above, wherein at least one hydrogen atom is replaced by an alkoxy group as defined herein, for example —CH₂CN, —CH₂CH₂CN, and the like.

The term “lower alkoxy” denotes a group having an alkyl group as defined above that is attached via an oxygen atom, for example, methoxy, ethoxy, propoxy, isopropoxy, n-butoxy, i-butoxy, 2-butoxy, t-butoxy and the like. Preferred alkoxy groups are groups with 1-4 carbon atoms.

The term “lower alkoxy substituted by halogen” denotes an alkoxy group as defined above wherein at least one hydrogen atom on the alkyl residue is replaced by a halogen atom. Preferred lower alkoxy substituted by halogen groups are groups having 1-4 carbon atoms.

The term“(C₃-C₇)-cycloalkyl” denotes a saturated carbocyclic group, containing 3 to 7 carbon atoms. For example, a cycloalkyl group is cyclopropyl, cyclobutyl, cyclopentyl, cyclohexyl and cycloheptyl, and these groups may optionally be substituted by one or two (C₁-C₄)-alkyl substituents, for example methyl or ethyl.

“Pharmaceutically acceptable,” such as pharmaceutically acceptable carrier, excipient, etc., means pharmacologically acceptable and substantially non-toxic to the subject to which the particular compound is administered.

The term “pharmaceutically acceptable acid addition salt” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methanesulfonic acid, p-toluenesulfonic acid and the like.

“Therapeutically effective amount” means an amount that is effective to prevent, alleviate or ameliorate symptoms of disease or prolong the survival of the subject being treated.

The following groups of compounds of formula I are preferred:

Preferred are compounds of formula I, wherein Ar¹ and Ar² are both phenyl.

Especially preferred from this group are compounds, wherein
—R¹ is methyl and R² is S(O)₂CH₃, for example
2-(3,4-dichloro-phenyl)-N-(3-fluoro-4-trifluoromethyl-benzyl)-4-[4-(methanesulfonyl-methyl-amino)-piperidin-1 -yl]-2,N-dimethyl-butyramide,
N-(4-chloro-3-trifluoromethyl-benzyl)-2-(3,4-dichloro-phenyl)-4-[4-(methanesulfonyl-methyl-amino)-piperidin-1-yl]-2,N-dimethyl-butyramide,
2-(3,4-dichloro-phenyl)-N-(4-fluoro-3-trifluoromethyl-benzyl)-4-[4-(methanesulfonyl-methyl-amino)-piperidin-1 -yl]-2,N-dimethyl-butyramide,
N-(3,4-dichloro-benzyl)-2-(3,4-dichloro-phenyl)-4-[4-(methanesulfonyl-methyl-amino)-piperidin-1-yl]-2,N-dimethyl-butyramide, and
2-(3,4-dichloro-phenyl)-N-[1-(3,4-dichloro-phenyl)-2-hydroxy-ethyl]-4-[4-(methanesulfonyl-methyl-amino)-piperidin-1-yl]-2-methyl-butyramide; diastereoisomer 1.

—R¹ is cyclopropyl and R² is S(O)₂CH₃, for example
4-[4-(cyclopropyl-methanesulfonyl-amino)-piperidin-1 -yl]-2-(3,4-dichloro-phenyl)-N-(3-fluoro-4-trifluoromethyl-benzyl)-2,N-dimethyl-butyramide,
N-(4-chloro-3-trifluoromethyl-benzyl)-4-[4-(cyclopropyl-methanesulfonyl-amino)-piperidin-1-yl]-2-(3,4-dichloro-phenyl)-2,N-dimethyl-butyramide,
4-[4-(cyclopropyl-methanesulfonyl-amino)-piperidin-1-yl]-2-(3,4-dichloro-phenyl)-N-(4-fluoro-3-trifluoromethyl-benzyl)-2,N-dimethyl-butyramide, and
4-[4-(cyclopropyl-methanesulfonyl-amino)-piperidin-1-yl]-N-(3,4-dichloro-benzyl)-2-(3,4-dichloro-phenyl)-2,N-dimethyl-butyramide.

—R¹ is ethyl and R² is S(O)₂CH₃, for example
2-(3,4-dichloro-phenyl)-4-[4-(ethyl-methanesulfonyl-amino)-piperidin-1-yl]-N-(3-fluoro-4-trifluoromethyl-benzyl)-2,N-dimethyl-butyramide,
N-(3,4-dichloro-benzyl)-2-(3,4-dichloro-phenyl)-4-[4-(ethyl-methanesulfonyl-amino)-piperidin-1-yl]-2,N-dimethyl-butyramide,
N-(3,4-dichloro-benzyl)-2-(3,4-dichloro-phenyl)-4-[4-(ethyl-methanesulfonyl-amino)-piperidin-1-yl]-2-methyl-butyramide,
N-(4-chloro-benzyl)-2-(3,4-dichloro-phenyl)-4-[4-(ethyl-methanesulfonyl-amino)-piperidin-1-yl]-2,N-dimethyl-butyramide,
N-(4-chloro-3-trifluoromethyl-benzyl)-2-(3,4-dichloro-phenyl)-4-[4-(ethyl-methanesulfonyl-amino)-piperidin-1-yl]-2,N-dimethyl-butyramide,
2-(3,4-dichloro-phenyl)-4-[4-(ethyl-methanesulfonyl-amino)-piperidin-1-yl]-N-(4-fluoro-3-trifluoromethyl-benzyl)-2,N-dimethyl-butyramide,
N-(3-chloro-benzyl)-2-(3,4-dichloro-phenyl)-4-[4-(ethyl-methanesulfonyl-amino)-piperidin-1-yl]-2,N-dimethyl-butyramide,
2-(3,4-dichloro-phenyl)-4-[4-(ethyl-methanesulfonyl-amino)-piperidin-1-yl]-2,N-dimethyl-N-(4-trifluoromethyl-benzyl)-butyramide,
2-(3,4-dichloro-phenyl)-4-[4-(ethyl-methanesulfonyl-amino)-piperidin-1-yl]-2,N-dimethyl-N-(4-methyl-benzyl)-butyramide,
N-(2-chloro-benzyl)-2-(3,4-dichloro-phenyl)-4-[4-(ethyl-methanesulfonyl-amino)-piperidin-1-yl]-2,N-dimethyl-butyramide,
2-(3,4-dichloro-phenyl)-N-[1-(3,4-dichloro-phenyl)-2-hydroxy-ethyl]-4-[4-(ethyl-methanesulfonyl-amino)-piperidin-1-yl]-2-methyl-butyramide; diastereoisomer 1.

—R¹ and R² form together with the N-atom to which they are attached a pyrrolidin-2-one group, for example
2-(3,4-dichloro-phenyl)-N-(3-fluoro-4-trifluoromethyl-benzyl)-2,N-dimethyl-4-[4-(2-oxo-pyrrolidin-1-yl)-piperidin-1-yl]-butyramide,
N-(3,4-dichloro-benzyl)-2-(3,4-dichloro-phenyl)-2,N-dimethyl-4-[4-(2-oxo-pyrrolidin-1yl)-piperidin-1-yl]-butyramide,
N-(3,4-dichloro-benzyl)-2-(3,4-dichloro-phenyl)-2-methyl-4-[4-(2-oxo-pyrrolidin-1-yl)-piperidin-1-yl]-butyramide,
N-(4-chloro-benzyl)-2-(3,4-dichloro-phenyl)-2,N-dimethyl-4-[4-(2-oxo-pyrrolidin-1-yl)-piperidin-1-yl]-butyramide,
N-(4-chloro-3-trifluoromethyl-benzyl)-2-(3,4-dichloro-phenyl)-2,N-dimethyl-4-[4-(2-oxo-pyrrolidin-1-yl)-piperidin-1-yl]-butyramide,
2-(3,4-dichloro-phenyl)-N-(4-fluoro-3-trifluoromethyl-benzyl)-2,N-dimethyl-4-[4-(2-oxo-pyrrolidin-1-yl)-piperidin-1-yl]-butyramide,
N-(3-chloro-benzyl)-2-(3,4-dichloro-phenyl)-2,N-dimethyl-4-[4-(2-oxo-pyrrolidin-1-yl)-piperidin-1-yl]-butyramide,
2-(3,4-dichloro-phenyl)-2,N-dimethyl-4-[4-(2-oxo-pyrrolidin-1-yl)-piperidin-1-yl]-N-(4-trifluoromethyl-benzyl)-butyramide,
2-(3,4-dichloro-phenyl)-2,N-dimethyl-N-(4-methyl-benzyl)-4-[4-(2-oxo-pyrrolidin-1-yl)-piperidin-1-yl]-butyramide,
N-(2-chloro-benzyl)-2-(3,4-dichloro-phenyl)-2,N-dimethyl-4-[4-(2-oxo-pyrrolidin-1-yl)-piperidin-1-yl]-butyramide,
2-(3,4-dichloro-phenyl)-N-[-1-(3,4-dichloro-phenyl)-2-hydroxy-ethyl]-2-methyl-4-[4-(2-oxo-pyrrolidin-1-yl)-piperidin-1-yl]-butyramide; diastereoisomer 1,
2-(3,4-dichloro-phenyl)-2-methyl-4-[4-(2-oxo-pyrrolidin-1-yl)-piperidin-1-yl]-N-(2,2,2-trifluoro-1-phenyl-ethyl)-butyramide,
2-(3,4-dichloro-phenyl)-2-methyl-4-[4-(2-oxo-pyrrolidin-1-yl)-piperidin-1-yl]-N-[2,2,2-trifluoro-1-(4-fluoro-phenyl)-ethyl]-butyramide,
N-(4-chloro-benzyl)-2-(3,4-dichloro-phenyl)-N-methyl-4-[4-(2-oxo-pyrrolidin-1-yl)-piperidin-1-yl]-butyramide,
(R or S)-2-(3,4-dichloro-phenyl)-N-(4-fluoro-3-trifluoromethyl-benzyl)-2,N-dimethyl-4-[4-(2-oxo-pyrrolidin-1-yl)-piperidin-1-yl]-butyramide,
(R or S)-2-(3,4-dichloro-phenyl)-N-[(S or R)-1-(3,4-dichloro-phenyl)-2-hydroxy-ethyl]-2-methyl-4-[4-(2-oxo-pyrrolidin-1-yl)-piperidin-1-yl]-butyramide,
2-(3,4-dichloro-phenyl)-2-methyl-4-[4-(2-oxo-pyrrolidin-1-yl)-piperidin-1-yl]-N-((S)-1-phenyl-propyl)-butyramide,
2-(3,4-dichloro-phenyl)-N-[(R)-1-(4-fluoro-phenyl)-2-hydroxy-ethyl]-2-(R or S) methyl-4-[4-(2-oxo-pyrrolidin-1-yl)-piperidin-1-yl]-butyramide, and
2-(3,4-dichloro-phenyl)-N-(4-fluoro-3-trifluoromethyl-benzyl)-2-methyl-4-[4-(2-oxo-pyrrolidin-1-yl)-piperidin-1-yl]-butyramide.

—R¹ is lower alkyl and R² is C(O)CH₃, for example
4-[4-(acetyl-methyl-amino)-piperidin-1-yl]-2-(3,4-dichloro-phenyl)-N-(3-fluoro-4-trifluoromethyl-benzyl)-2-methyl-butyramide,
4-[4-(acetyl-methyl-amino)-piperidin-1-yl]-N-(3,4-dichloro-benzyl)-2-(3,4-dichloro-phenyl)-2,N-dimethyl-butyramide,
4-[4-(acetyl-methyl-amino)-piperidin-1-yl]-N-(3,4-dichloro-benzyl)-2-(3,4-dichloro-phenyl)-2-methyl-butyramide,
4-[4-(acetyl-methyl-amino)-piperidin-1-yl]-N-(4-chloro-benzyl)-2-(3,4-dichloro-phenyl)-2,N-dimethyl-butyramide,
4-[4-(acetyl-methyl-amino)-piperidin-1-yl]-N-(4-chloro-3-trifluoromethyl-benzyl)-2-(3,4-dichloro-phenyl)-2,N-dimethyl-butyramide,
4-[4-(acetyl-methyl-amino)-piperidin-1-yl]-2-(3,4-dichloro-phenyl)-N-(4-fluoro-3-trifluoromethyl-benzyl)-2,N-dimethyl-butyramide,
4-[4-(acetyl-methyl-amino)-piperidin-1-yl]-N-(3-chloro-benzyl)-2-(3,4-dichloro-phenyl)-2,N-dimethyl-butyramide,
4-[4-(acetyl-methyl-amino)-piperidin-1-yl]-2-(3,4-dichloro-phenyl)-2,N-dimethyl-N-(4-trifluoromethyl-benzyl)-butyramide,
4-[4-(acetyl-methyl-amino)-piperidin-1-yl]-2-(3,4-dichloro-phenyl)-N-[1-(3,4-phenyl)-2-hydroxy-ethyl]-2-methyl-butyramide; diastereoisomer 1,
(R or S)-4-[4-(acetyl-methyl-amino)-piperidin-1-yl]-2-(3,4-dichloro-phenyl)-N-(4-fluoro-3-trifluoromethyl-benzyl)-2,N-dimethyl-butyramide,
4-[4-(acetyl-methyl-amino)-piperidin-1-yl]-N-[1 -(4-chloro-phenyl)-2-hydroxy-ethyl]-2-(3,4-dichloro-phenyl)-2-methyl-butyramide (diastereosisomer 1),
4-[4-(acetyl-propyl-amino)-piperidin-1-yl]-2-(3,4-dichloro-phenyl)-N-[(R)-1-(4-fluoro-phenyl)-2-hydroxy-ethyl]-2-methyl-butyramide (epimer 1),
4-[4-(acetyl-propyl-amino)-piperidin-1-yl]-2-(3,4-dichloro-phenyl)-N-[(R)-1-(4-fluoro-phenyl)-2-hydroxy-ethyl]-2-methyl-butyramide (epimer 2),
4-[4-(acetyl-propyl-amino)-piperidin-1-yl]-2-(3,4-dichloro-phenyl)-2-methyl-N-((S)-1-phenyl-propyl)-butyramide,
4-[4-(acetyl-ethyl-amino)-piperidin-1-yl]-2-(3,4-dichloro-phenyl)-N-[(R)-1-(4-fluoro-phenyl)-2-hydroxy-ethyl]-2-methyl-butyramide (epimer 1), and
4-[4-(acetyl-ethyl-amino)-piperidin-1-yl]-2-(3,4-dichloro-phenyl)-2-methyl-N-((S)-1-phenyl-propyl)-butyramide.

—R¹ is —(CH₂)₂OCH₃ and R² is C(O)CH₃, for example
4-{4-[acetyl-(2-methoxy-ethyl)-amino]-piperidin-1-yl}-2-(3,4-dichloro-phenyl)-N-[1-(4-fluoro-phenyl)-2-hydroxy-ethyl]-2-methyl-butyramide (diastereoisomer 1),
4-{4-[acetyl-(2-methoxy-ethyl)-amino]-piperidin-1-yl}-2-(3,4-dichloro-phenyl)-N-[1-( 3,4-dichloro-phenyl)-2-hydroxy-ethyl]-2-methyl-butyramide (diastreoisomer 1),
4-{4-[acetyl-(2-methoxy-ethyl)-amino]-piperidin-1-yl}-2-(3,4-dichloro-phenyl)-N-[(R)-1-(4-fluoro-phenyl)-2-hydroxy-ethyl]-2-(R or S) methyl-butyramide, and
4-{4-[acetyl-(2-methoxy-ethyl)-amino]-piperidin-1-yl}-2-(3,4-dichloro-phenyl)-2-methyl-N-((S)-1-phenyl-propyl)-butyramide.

The preparation of compounds of formula I of the present invention can be carried out in sequential or convergent synthetic routes. Syntheses of the compounds of the invention are shown in the following scheme 1. The skills required for carrying out the reaction and purification of the resulting products are known to those skilled in the art. The substituents and indices used in the following description of the processes have the significance given herein before unless indicated to the contrary.

In more detail, the compounds of formula I can be manufactured by the methods given below, by the methods given in the examples or by analogous methods. Appropriate reaction conditions for the individual reaction steps are known to a person skilled in the art. The reaction sequence is not limited to the one displayed in scheme 1, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

The present compounds of formula I and their pharmaceutically acceptable salts can be prepared by processes described below, which processes comprise
a) cleaving off an O-protecting group under aqueous basic conditions from a compound of formula IV

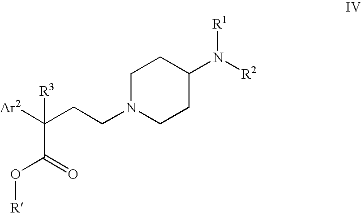

and reacting a compound of formula IV under coupling conditions with an amine of formula

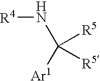

to obtain a compound of formula

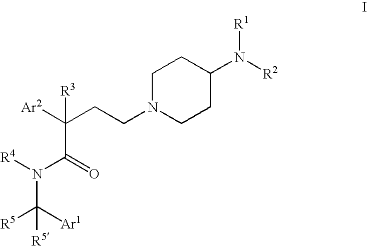

wherein the definitions have same meanings as described above, and
if desired, converting the compounds obtained into pharmaceutically acceptable acid addition salts.

The process is described in scheme 1 in more detail.

### General Experimental Part

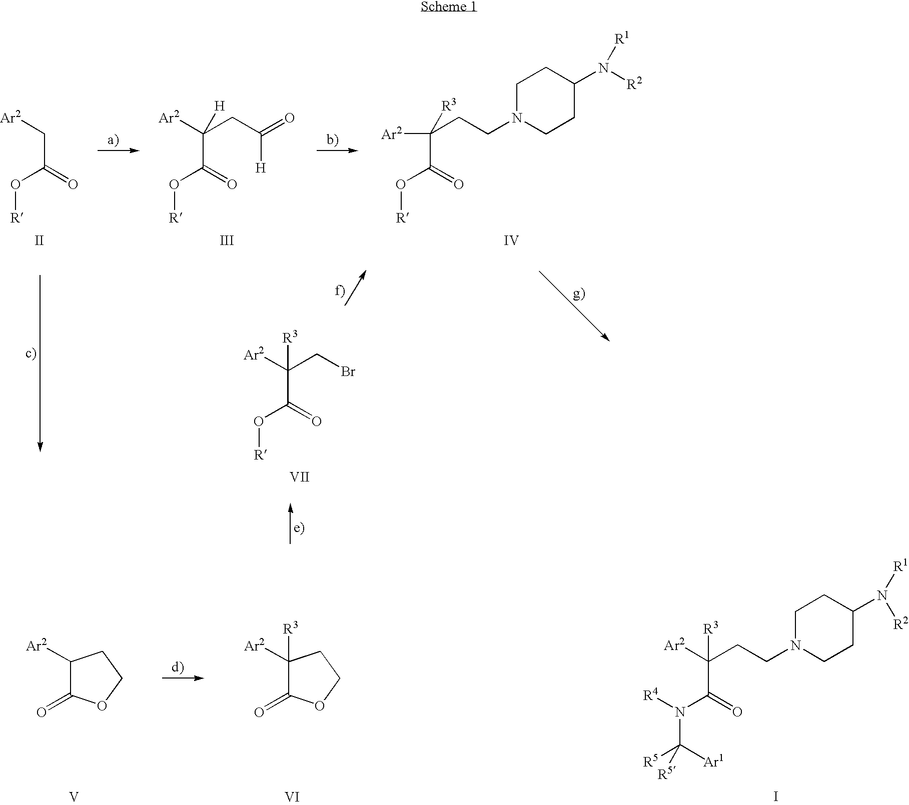

R¹, R², R³, R⁴, R⁵, R^{5′}, Ar¹ and Ar² are as described above and R′ is an O-protecting group, such as benzyl.

### Step a)

Phenylacetic acid ester derivatives II are commercially available or can be accessed by methods described in literature. Reaction of ester derivatives II with protected bromo alkyl aldehydes (either commercially available or synthetically accessible by methods known in the art) under basic conditions lead to aldehyde derivatives III as described analogously in literature (for reaction conditions described in literature affecting such reactions see for example: Comprehensive Organic Transformations: A Guide to Functional Group Preparations, 2nd Edition, Richard C. Larock. John Wiley & Sons, New York, N.Y. 1999). However, it is convenient to react ester derivative II with the respective protected bromo alkyl aldehyde (commercially available or accessible by methods known) in the presence of a base and a solvent. There is no particular restriction on the nature of the solvent to be employed, provided that it has no adverse effect on the reaction or the reagents involved and that it can dissolve the reagents, at least to some extent. Examples for suitable solvents include dimethylformamide (DMF), tetrahydrofuran (THF) and the like. There is no particular restriction on the nature of the base used in this stage, and any base commonly used in this type of reaction may equally be employed here. Examples of such bases include NaH and the like. The reaction can take place over a wide range of temperatures, and the precise reaction temperature is not critical to the invention. It is convenient to carry out the reaction with heating from ambient temperature to reflux. The time required for the reaction may also vary widely, depending on many factors, notably the reaction temperature and the nature of the reagents. However, a period of from 0.5 h to several days will usually suffice to yield the aldehyde protected intermediate which can be subjected to acidic cleavage of the protecting group in the presence of a solvent. There is no particular restriction on the nature of the solvent to be employed, provided that it has no adverse effect on the reaction or the reagents involved and that it can dissolve the reagents, at least to some extent. Examples for suitable solvents include tetrahydrofuran (THF) and the like. There is no particular restriction on the nature of the acid used in this stage, and any acid commonly used in this type of reaction may equally be employed here. Examples of such acid include HCl and the like. The reaction can take place over a wide range of temperatures, and the precise reaction temperature is not critical to the invention. It is convenient to carry out the reaction with heating from ambient temperature to reflux. The time required for the reaction may also vary widely, depending on many factors, notably the reaction temperature and the nature of the reagents. However, a period of from 0.5 h to several days will usually suffice to yield aldehyde derivatives III.

### Step b)

Reductive aminations are widely described in literature (for reaction conditions described in literature affecting such reactions see for example: Comprehensive Organic Transformations: A Guide to Functional Group Preparations, 2nd Edition, Richard C. Larock. John Wiley & Sons, New York, N.Y. 1999). However, we find it convenient to transform aldehyde derivative III with piperidine derivatives (Journal of Medicinal Chemistry (2006), 49(16), 4801-4804) under reductive conditions in the presence of a solvent to afford ester derivatives IV. There is no particular restriction on the nature of the solvent to be employed, provided that it has no adverse effect on the reaction or the reagents involved and that it can dissolve the reagents, at least to some extent. Examples for suitable solvents include tetrahydrofuran (THF) and the like. There is no particular restriction on the nature of the reducing agent used in this stage, and any reducing agent commonly used in this type of reaction may equally be employed here. Examples of such reducing agents include sodium triacetoxyborohydride and the like. The reaction can take place over a wide range of temperatures, and the precise reaction temperature is not critical to the invention. It is convenient to carry out the reaction with heating from ambient temperature to reflux. The time required for the reaction may also vary widely, depending on many factors, notably the reaction temperature and the nature of the reagents. However, a period of from 0.5 h to several days will usually suffice to yield ester derivatives IV.

### Step c)

Reaction of ester derivatives II with hydroxy-protected alkyl halides (either commercially available or synthetically accessible by methods known in the art) under basic conditions lead upon cleavage of the hydroxyl protecting group to lactones V as described in literature (for reaction conditions described in literature affecting such reactions see for example: Comprehensive Organic Transformations: A Guide to Functional Group Preparations, 2nd Edition, Richard C. Larock. John Wiley & Sons, New York, N.Y. 1999). However, it is convenient to react ester derivative II with 2-(2-bromoethoxy) tetrahydro-2-H-pyrane (commercially available in the presence of a base and a solvent. There is no particular restriction on the nature of the solvent to be employed, provided that it has no adverse effect on the reaction or the reagents involved and that it can dissolve the reagents, at least to some extent. Examples for suitable solvents include dimethylformamide (DMF), tetrahydrofuran (THF) and the like. There is no particular restriction on the nature of the base used in this stage, and any base commonly used in this type of reaction may equally be employed here. Examples of such bases include NaH and the like. The reaction can take place over a wide range of temperatures, and the precise reaction temperature is not critical to the invention. It is convenient to carry out the reaction with heating from ambient temperature to reflux. The time required for the reaction may also vary widely, depending on many factors, notably the reaction temperature and the nature of the reagents. However, a period of from 0.5 h to several days will usually suffice to yield the hydroxy protected intermediate which can be subjected to acidic cleavage of the protecting group in the presence of a solvent. There is no particular restriction on the nature of the solvent to be employed, provided that it has no adverse effect on the reaction or the reagents involved and that it can dissolve the reagents, at least to some extent. Examples for suitable solvents include dimethylformamide (DMF), tetrahydrofuran (THF) and the like. There is no particular restriction on the nature of the acid used in this stage, and any acid commonly used in this type of reaction may equally be employed here. Examples of such acid include HCl and the like. The reaction can take place over a wide range of temperatures, and the precise reaction temperature is not critical to the invention. It is convenient to carry out the reaction with heating from ambient temperature to reflux. The time required for the reaction may also vary widely, depending on many factors, notably the reaction temperature and the nature of the reagents. However, a period of from 0.5 h to several days will usually suffice to yield lactone derivatives V.

### Step d)

Lactone derivatives V can conveniently be transferred into the respective substituted lactone derivatives VI by reaction of lactone V with an electrophile in the presence of a base in the presence of a solvent. There is no particular restriction on the nature of the solvent to be employed, provided that it has no adverse effect on the reaction or the reagents involved and that it can dissolve the reagents, at least to some extent. Examples for suitable solvents include dimethylformamide (DMF), tetrahydrofuran (THF) and the like. There is no particular restriction on the nature of the base used in this stage, and any base commonly used in this type of reaction may equally be employed here. Examples of such bases include NaH and the like. The reaction can take place over a wide range of temperatures, and the precise reaction temperature is not critical to the invention. It is convenient to carry out the reaction with heating from ambient temperature to reflux. The time required for the reaction may also vary widely, depending on many factors, notably the reaction temperature and the nature of the reagents. However, a period of from 0.5 h to several days will usually suffice to yield lactone derivatives VI.

### Step e)

Lactone derivative VI can conveniently be transferred into the respective ester derivative VII by a two step reaction sequence. Any commonly used synthetic sequence is applicable however, we find it convenient to open the lactone derivative VII with HBr in the presence of an acid. Any commonly used acid which in combination with HBr affects such a reaction can be used. Examples of such acids include acetic acid and the like. The reaction can take place over a wide range of temperatures, and the precise reaction temperature is not critical to the invention. It is convenient to carry out the reaction with heating from ambient temperature to reflux. The time required for the reaction may also vary widely, depending on many factors, notably the reaction temperature and the nature of the reagents. However, a period of from 0.5 h to several days will usually suffice to yield the intermediately built acid derivative which is subjected to esterification conditions. Common procedures are described in literature, however, we find it convenient to transform the intermediately built acid into the respective ester derivative VII by reaction with SOCl₂ in methanol. The reaction can take place over a wide range of temperatures, and the precise reaction temperature is not critical to the invention. It is convenient to carry out the reaction with heating from ambient temperature to reflux. The time required for the reaction may also vary widely, depending on many factors, notably the reaction temperature and the nature of the reagents. However, a period of from 0.5 h to several days will usually suffice to yield ester derivative VII.

### Step f)

Transformation of ester derivatives VII with piperidine derivatives to access piperidine derivatives IV can be done by any commonly used procedure. However, we find it convenient to react ester derivative VII with piperidine derivatives in the presence of a solvent and a base. There is no particular restriction on the nature of the solvent to be employed, provided that it has no adverse effect on the reaction or the reagents involved and that it can dissolve the reagents, at least to some extent. Examples for suitable solvents include dimethylformamide (DMF), tetrahydrofuran (THF) and the like. There is no particular restriction on the nature of the base used in this stage, and any base commonly used in this type of reaction may equally be employed here. Examples of such bases include DIPEA, NEt₃ and the like. The reaction can take place over a wide range of temperatures, and the precise reaction temperature is not critical to the invention. It is convenient to carry out the reaction with heating from ambient temperature to reflux. The time required for the reaction may also vary widely, depending on many factors, notably the reaction temperature and the nature of the reagents. However, a period of from 0.5 h to several days will usually suffice to yield piperidine derivatives IV.

### Step g)

Transformation of piperidine derivatives IV into the final amide derivatives can be done according to procedures described in literature. However, we find it convenient to employ a two step reaction sequence in which the ester functionality in IV is cleaved under aqueous basic conditions and the liberated acid functionality converted with the respective amines under coupling conditions and to the piperidine derivatives I. There is no particular restriction on the nature of the aqueous base to be employed, provided that it has no adverse effect on the reaction or the reagents involved and that it can dissolve the reagents, at least to some extent. Examples for suitable aqueous bases include NaOH, LiOH and the like. Any commonly used co-solvent can be employed. Examples include THF and the like. The coupling of carboxylic acids with amines is widely described in literature and the procedures are known to those in the art (For reaction conditions described in literature affecting such reactions see for example: Comprehensive Organic Transformations: A Guide to Functional Group Preparations, 2nd Edition, Richard C. Larock. John Wiley & Sons, New York, N.Y. 1999). The intermediately built acid can conveniently be transformed to the respective amide through coupling with an amine (either commercially available or accessible by methods described in references or by methods known in the art; as appropriate) by employing the usage of coupling reagents. For example coupling reagents like N,N′-carbonyldiimidazole (CDI), N,N′-dicyclohexylcarbodiimide (DCC), 1-(3-dimethylaminopropyl)-3-ethylcarbodiimide hydrochloride (EDCI), 1-[bis(dimethylamino)methylene]-1H-1,2,3-triazolo [4,5-b]pyridinium-3-oxide hexafluorophosphate (HATU), 1-hydroxy-1,2,3-benzotriazole (HOBT), O-benzotriazol-1-yl-N,N,N′,N′-tetramethyluronium tetrafluoroborate (TBTU) and the like can equally well be employed to affect such transformation. We find it convenient to carry out the reaction in a solvent like dimethylformamide (DMF) and in the presence of a base. There is no particular restriction on the nature of the solvent to be employed, provided that it has no adverse effect on the reaction or the reagents involved and that it can dissolve the reagents, at least to some extent. Examples for suitable solvents include: DMF, dichloromethane (DCM), dioxane, THF, and the like. There is no particular restriction on the nature of the base used in this stage, and any base commonly used in this type of reaction may equally be employed here. Examples of such bases include triethylamine and diisopropylethylamine, and the like. The reaction can take place over a wide range of temperatures, and the precise reaction temperature is not critical to the invention. We find it convenient to carry out the reaction with heating from ambient temperature to reflux. The time required for the reaction may also vary widely, depending on many factors, notably the reaction temperature and the nature of the reagents. However, a period of from 0.5 h to several days will usually suffice to yield piperidine derivatives I.

Where examples are said to be performed in analogy to a specific procedure, it is understood that variables within the synthesis may be optimized for the specific compound produced in accordance with knowledge within the level of skill in the art.

### EXAMPLES

### Intermediate 1

### 2-(3,4-dichloro-phenyl)-4-(4-methanesulfonylamino-piperidin-1-yl)-2-methyl-butyric acid

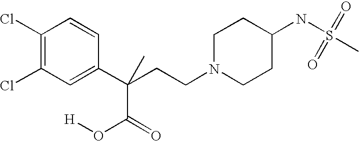

### a) Step 1:

### 3-(3,4-Dichloro-phenyl)-dihydro-furan-2-one (commercially available)

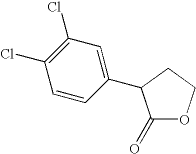

A mixture of 30.00 g (137 mmol) (3,4-dichloro-phenyl)-acetic acid methyl ester (commercially available), 6.47 g (151 mmol) NaH (55%) and 35.80 g (171 mmol) 2-(2-bromo-ethoxy)-tetrahydro-pyran in 100 mL DMF was stirred at room temperature for 17 h. The mixture was evaporated to dryness and partitioned between water and ethyl acetate. The combined organic phases were washed with NaCl aq., dried with Na₂SO₄ and evaporated. The residue was treated with 400 mL 4N HCl in dioxane and stirred for 16 h at room temperature. The mixture was evaporated to dryness and subjected to column chromatography on silica eluting with a gradient formed from ethyl acetate and heptane. The combined product fractions were evaporated to yield 18.5 g (58%) of the title compound as yellow oil.

### b) Step 2:

### 3-(3,4-Dichloro-phenyl)-3-methyl-dihydro-furan-2-one

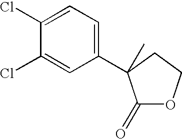

A mixture of 18.50 g (80 mmol) 3-(3,4-dichloro-phenyl)-dihydro-furan-2-one, 3.84 g (88 mmol) NaH (55% suspension) and 14.20 g (100 mmol) iodomethane in 300 mL THF was stirred for 64 h at room temperature. NH₄Cl aq. sat. was added and the mixture was extracted with ethyl acetate. The organic phases were washed with NaCl aq. sat. dried with Na₂SO₄, filtered and evaporated to dryness. The residue was purified by flash column chromatography on silica eluting with a gradient formed from ethyl acetate and heptane. The product containing fractions were evaporated to yield 16 g (82%) of the title compound as yellow oil. MS(m/e): 246.0 (MH⁺).

### c) Step 3:

### 4-Bromo-2-(3,4-dichloro-phenyl)-2-methyl-butyric acid methyl ester

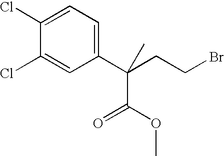

To a mixture of 3.30 g (13.5 mmol) 3-(3,4-dichloro-phenyl)-3-methyl-dihydro-furan-2-one in 15 mL acetic acid was added 48 mL HBr (33%) in acetic acid and after 63 h 20 mL HBr (33%) in acetic acid was added again and stirred for another 21 h at room temperature. The mixture was pored onto ice-water and extracted with ethyl ether. The combined organic phases were washed with NaCl aq. sat., dried with Na₂SO₄, filtered and evaporated to dryness, The residue was taken up in 150 mL toluene and 6.50 mL (89.0 mmol) thionylchloride were added. The mixture was heated to 75° C. for 4 h, cooled to 0° C., treated with 20 mL methanol and allowed to stand for 16 h at room temperature. The mixture was evaporated to dryness and subjected to column chromatography on silica eluting with a gradient formed from ethyl acetate and heptane. The product containing fractions were evaporated to yield 4.32 g (94%) of the title compound as light yellow oil. MS(m/e): 341.9 (MH⁺).

### d) Step 4:

### 2-(3,4-dichloro-phenyl)-4-(4-methanesulfonylamino-piperidin-1-yl)-2-methyl-butyric acid methyl ester

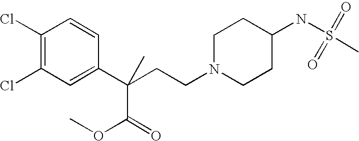

A mixture of 0.70 g (2.05 mmol) 4-bromo-2-(3,4-dichloro-phenyl)-2-methyl-butyric acid methyl ester and 1.83 g (10.30 mmol) N-piperidin-4-yl-methanesulfonamide (commercially available) in 30 mL N,N-dimethylacetamide was stirred for 89 h at 60° C. The mixture was evaporated to dryness and methanol, DCM and isolute was added and evaporated to dryness. The residue was subjected to column chromatography on silica eluting with a gradient formed from DCM, methanol and NH₃ aq. The product containing fractions were evaporated to yield 0.76 g (84%) of the title compound as light brown waxy solid. MS(m/e): 437.3 (MH⁺).

### e) Step 5:

A mixture of 0.76 g (1.7 mmol) 2-(3,4-dichloro-phenyl)-4-(4-methanesulfonylamino-piperidin-1-yl)-2-methyl-butyric acid methyl ester and 0.23 g (5.4 mmol) LiOH.H₂O in 15 mL water and 15 mL THF was stirred at room temperature for 18 h. The mixture was evaporated and treated with 4N HCl aq. and evaporated to dryness. The residue was used without further purification in the subsequent step. MS(m/e): 423.1 (MH⁺).

### Intermediate 2

### 2-(3,4-Dichloro-phenyl)-4-(4-methanesulfonylamino-piperidin-1-yl)-butyric acid

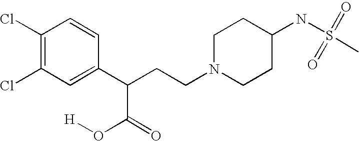

### a) Step 1:

### 2-(3,4-Dichloro-phenyl)-4-oxo-butyric acid methyl ester

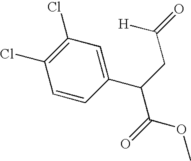

A mixture of 25.0 g (114 mmol) (3,4-dichloro-phenyl)-acetic acid methyl ester (commercially available), 5.7 g (131 mmol) NaH (55%) and 23.1 g (137 mmol) bromoacetaldehyde dimethylacetal in 80 mL DMF was stirred at room temperature for 3 h. The mixture was poured onto ice/water and extracted with ethyl acetate. The combined organic phases were washed with NaCl aq., dried with Na₂SO₄ and evaporated to dryness. The residue was dissolved in 250 mL THF and treated with 300 mL IN HCl at room temperature for 20 h. Water was added and the mixture was extracted with ethyl acetate. The combined organic phases were washed with NaCl aq., dried with Na₂SO₄, evaporated to dryness and subjected to column chromatography on silica eluting with a gradient formed from heptane and ethyl acetate. The product containing fractions were evaporated to yield 9.7 g (32%) of the title compound as light yellow oil. MS(m/e): 260.1/262.2 (MH⁺)

### b) Step 2:

### 2-(3,4-Dichloro-phenyl)-4-(4-methanesulfonylamino-piperidin-1-yl)-butyric acid methyl ester

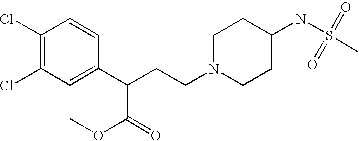

A mixture of 0.89 g (3.4 mmol) 2-(3,4-dichloro-phenyl)-4-oxo-butyric acid methyl ester, 0.67 g (3.7 mmol) N-Piperidin-4-yl-methanesulfonamide (commercially available), 1.08 g (5.0 mmol) sodium triacteoxyborohydride and 0.30 g (5.0 mmol) acetic acid in 40 mL THF was stirred at room temperature for 64 h. Water and Na₂CO₃ aq. was added and the mixture was extracted with ethyl acetate. The combined organic fractions were washed with NaCl sat. aq. dried with Na₂SO₄, filtered and evaporated to dryness. The residue was purified by flash column chromatography on silica eluting with a gradient formed from DCM, methanol and NH₃ aq. The product containing fractions were evaporated to yield 1.21 g (84%) of the title compound as off-white solid. MS(m/e): 423.1 (MH⁺).

### c) Step 3:

A mixture of 1.16 g (2.7 mmol) 2-(3,4-dichloro-phenyl)-4-(4-methanesulfonylamino-piperidin-1-yl)-butyric acid methyl ester and 0.35 g (8.2 mmol) LiOH.H₂O in 20 mL water and 20 mL THF was stirred for 69 h at room temperature and evaporated. The residue was treated with 4N HCl aq. and evaporated to dryness and dried in vacuo. The residue was used without further purification in the subsequent step. MS(m/e): 409.4 (MH⁺).

### Intermediate 3

### 2-(3,4-Dichloro-phenyl)-4-[4-(methanesulfonyl-methyl-amino)-piperidin-1-yl]-2-methyl-butyric acid

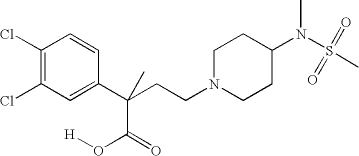

### a) Step 1:

### 2-(3,4-Dichloro-phenyl)-4-[4-(methanesulfonyl-methyl-amino)-piperidin-1-yl]-2-methyl-butyric acid methyl ester

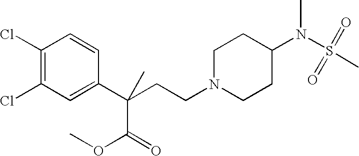

In analogy to the procedure described for the synthesis of 2-(3,4-dichloro-phenyl)-4-(4-methanesulfonylamino-piperidin-1-yl)-2-methyl-butyric acid methyl ester (intermediate 1, step 4) the title compound was prepared from 4-bromo-2-(3,4-dichloro-phenyl)-2-methyl-butyric acid methyl ester and N-methyl-N-piperidin-4-yl-methanesulfonamide (DE 2824064) as light yellow viscous oil. MS(m/e): 451.2 (MH⁺).

### b) Step 2:

In analogy to the procedure described for the synthesis of 2-(3,4-dichloro-phenyl)-4-(4-methanesulfonylamino-piperidin-1-yl)-2-methyl-butyric acid (intermediate 1, step 5) the title compound was prepared from 2-(3,4-dichloro-phenyl)-4-[4-(methanesulfonyl-methyl-amino)-piperidin-1-yl]-2-methyl-butyric acid methyl ester by saponification with LiOH.H₂O as off-white foam. MS(m/e): 437.1 (MH⁺).

### Intermediate 4

### 2-(3,4-Dichloro-phenyl)-2-fluoro-4-[4-(methanesulfonyl-methyl-amino)-piperidin-1-yl]-butyric acid

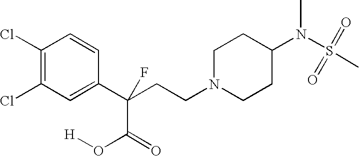

### a) Step 1:

### 3-(3,4-Dichloro-phenyl)-3-fluoro-dihydro-furan-2-one

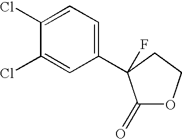

A mixture of 4.00 g (18.7 mmol) 3-(3,4-dichloro-phenyl)-dihydro-furan-2-one (commercially available), 0.83 g (19.0 mmol) NaH (55% suspension in oil) and 6.00 g (19.0 mmol ) N-fluorobenzenesulfonimide (commercially available) in 100 mL THF stirred at room temperature for 1 h. The mixture was filtered and the filtrate evaporated to dryness. DCM and isolute was added and the mixture was evaporated to dryness. The residues was subjected to column chromatography on silica eluting with a gradient formed from heptane and toluene. The product containing fraction were evaporated to yield 3.8 g (88%) of the title compound as colourless oil. MS(m/e): 248.1/250.1 /251.1 (MH⁺).

### b) Step 2:

### 4-Bromo-2-(3,4-dichloro-phenyl)-2-fluoro-butyric acid methyl ester

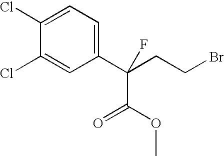

In analogy to the procedure described for the preparation of 4-bromo-2-(3,4-dichloro-phenyl)-2-methyl-butyric acid methyl ester (intermediate 1, step 3) the title compound was synthesized from 3-(3,4-dichloro-phenyl)-3-fluoro-dihydro-furan-2-one by acidic bromination with acetic acid and HBr and subsequent esterification with SOC1₂ and methanol. The title compound was obtained as colourless oil. MS(m/e): 342/346/348 (MH⁺).

### c) Step 3:

### 2-(3,4-Dichloro-phenyl)-2-fluoro-4-[4-(methanesulfonyl-methyl-amino)-piperidin-1-yl]-butyric acid methyl ester

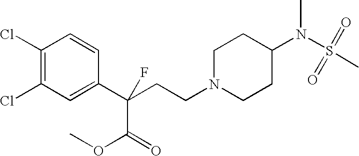

In analogy to the procedure described for the synthesis of 2-(3,4-dichloro-phenyl)-4-(4-methanesulfonylamino-piperidin-1-yl)-2-methyl-butyric acid methyl ester (intermediate 1, step 4) the title compound was prepared from 4-bromo-2-(3,4-dichloro-phenyl)-2-fluoro-butyric acid methyl ester and N-methyl-N-piperidin-4-yl-methanesulfonamide (DE 2824064) as light brown waxy solid. MS(m/e): 455.1 (MH⁺).

### d) Step 4:

In analogy to the procedure described for the synthesis of 2-(3,4-dichloro-phenyl)-4-(4-methanesulfonylamino-piperidin-1-yl)-2-methyl-butyric acid (intermediate 1, step 5) the title compound was prepared from 2-( 3,4-dichloro-phenyl)-2-fluoro-4-[4-(methanesulfonyl-methyl-amino)-piperidin-1-yl]-butyric acid methyl ester by saponification with LiOH.H₂O as off-white solid. MS(m/e): 441.1 (MH⁺).

### Intermediate 5

### N-Cyclopropyl-N-piperidin-4-yl-methanesulfonamide

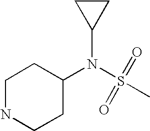

### a) Step 1:

### N-(-1-Benzyl-piperidin-4-yl)-N-cyclopropyl-methanesulfonamide

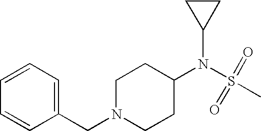

A mixture of 9.40 g (40.8 mmol) 1-benzyl-cyclopropylpiperidine-4-amine (commercially available), 5.14 g (44.8 mmol) methanesulfonyl chloride and 4.95 g (49.0 mmol) NEt₃ in 150 mL DCM was stirred for 17 h at room temperature. The mixture was evaporated to dryness and subjected to column chromatography on silica eluting with a gradient formed from DCM, methanol and NH₃ aq. The product containing fractions were evaporated to yield 11.40 g (90%) of the title compound as light yellow solid. MS(m/e): 309.3 (MH⁺).

### b) Step 2:

A mixture of 11.3 g (36.6 mmol) N-(1-benzyl-piperidin-4-yl)-N-cyclopropyl methane sulfonamide and 5.1 g Pd/C (10%) in 150 mL THF, 300 mL methanol and 31.7 mL formic acid was hydrogenated at room temperature. The mixture was filtered and evaporated to dryness. Water and Na₂CO₃ aq. was added and the mixture was extracted with THF/ethyl acetate. The combined organic phases were washed with NaCl aq. sat., dried with Na₂SO₄, filtered and evaporated to dryness. The residue was taken up in DCM and subjected to flash column chromatography on silica eluting with a gradient formed from DCM, methanol and NH₃ aq. The product containing fractions were evaporated to yield 5 g (62%) of the title compound as white solid. MS(m/e): 219.1 (MH⁺).

### Intermediate 6

### 4-[4-(Cyclopropyl-methanesulfonyl-amino)-piperidin-1-yl]-2-(3,4-dichloro-phenyl)-2-methyl-butyric acid

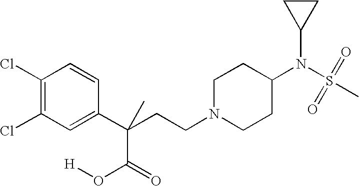

### a) Step 1:

### 4-[4-(Cyclopropyl-methanesulfonyl-amino)-piperidin-1-yl]-2-(3,4-dichloro-phenyl)-2-methyl-butyric acid methyl ester

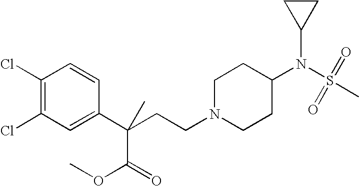

In analogy to the procedure described for the synthesis of 2-(3,4-dichloro-phenyl)-4-(4-methanesulfonylamino-piperidin-1-yl)-2-methyl-butyric acid methyl ester (intermediate 1, step 4) the title compound was prepared from 4-bromo-2-(3,4-dichloro-phenyl)-2-methyl-butyric acid methyl ester and N-cyclopropyl-N-piperidin-4-yl-methanesulfonamide as viscous colourless oil. MS(m/e): 476.9 (MH⁺).

### b) Step 2:

In analogy to the procedure described for the synthesis of 2-(3,4-dichloro-phenyl)-4-(4-methanesulfonylamino-piperidin-1-yl)-2-methyl-butyric acid (intermediate 1, step 5) the title compound was prepared from 4-[4-cyclopropyl-methanesulfonyl-amino)-piperidin-1-yl]-2-(3,4-dichloro-phenyl)-2-methyl-butyric acid methyl ester by saponification with LiOH.H₂O as white foam. MS(m/e): 446.1 (MH⁺).

### Intermediate 7

### 2-(3,4-Dichloro-phenyl)-2-fluoro-4-(4-methanesulfonylamino-piperidin-1-yl)-butyric acid

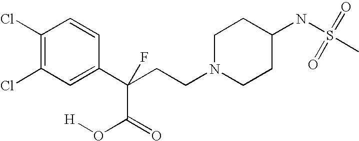

### a) Step 1:

### 2-(3,4-Dichloro-phenyl)-2-fluoro-4-(4-methanesulfonylamino-piperidin-1-yl)-butyric acid methyl ester

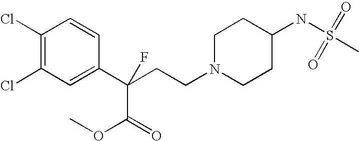

In analogy to the procedure described for the synthesis of 2-(3,4-dichloro-phenyl)-4-(4-methanesulfonylamino-piperidin-1-yl)-2-methyl-butyric acid methyl ester (intermediate 1, step 4) the title compound was prepared from 4-bromo-2-(3,4-dichloro-phenyl)-2-fluoro-butyric acid methyl ester and N-piperidin-4-yl-methanesulfonamide (commercially available) as light brown waxy solid. MS(m/e): 441.1 (MH⁺).

### b) Step 2:

In analogy to the procedure described for the synthesis of 2-(3,4-dichloro-phenyl)-4-(4-methanesulfonylamino-piperidin-1-yl)-2-methyl-butyric acid (intermediate 1, step 5) the title compound was prepared from 2-(3,4-dichloro-phenyl)-2-fluoro-4-(4-methanesulfonylamino-piperidin-1-yl)-butyric acid methyl ester by saponification with LiOH.H₂O and conversion with HCR into the respective salt as off-white solid. MS(m/e): 427.1 (MH⁺).

### Intermediate 8

### N-Ethyl-N-piperidin-4-yl-methane sulfonamide

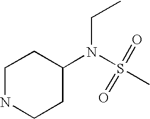

### a) Step 1:

### 4-Ethylamino-piperidine-1-carboxylic acid benzyl ester

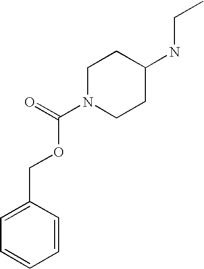

A mixture of 28.93 g (124 mmol) 1-(benzyloxycarbonyl)-4-piperidinone (commercially available, 8.30 g (186 mmol) ethylamine (2N in THF), 39.42 g (186 mmol) sodium triacetoxyborohydride and 11.10 g (186 mmol) acetic acid in 300 mL THF was stirred for 68 h at room temperature. Water and Na₂CO₃ aq. was added and extracted with ethyl acetate. The organic phases were washed with NaCl aq. sat., dried with Na₂SO₄, filtered and evaporated to dryness. The crude title compound was used in the consecutive step. MS(m/e): 263.0 (MH⁺).

### b) Step 2:

### 4-(Ethyl-methanesulfonyl-amino)-piperidine-1-carboxylic acid benzyl ester

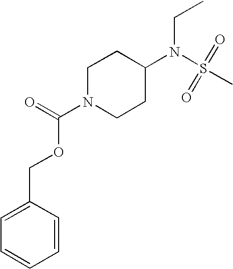

A mixture of 32.68 g (125 mmol) 4-ethylamino-piperidine-1-carboxylic acid benzyl ester, 16.00 g (140 mmol) methanesulfonyl chloride and 19.30 (149 mmol) DIPEA in 300 mL DCM was stirred for 80 min at room temperature. The mixture was concentrated, isolute was added and evaporated to dryness. The residue was subjected to flash column chromatography on silica eluting with a gradient formed from DCM, methanol and NH₃ aq. The product containing fractions were evaporated and the residue was triturated with a mixture formed from heptane and diethyl ether. The precipitate was filtered and dried to yield 32.4 g (76%) of the title compound as white solid. MS(m/e): 341.1 (MH⁺).

### c) Step 3:

A solution of 32.4 g (95 mmol) 4-(ethyl-methanesulfonyl-amino)-piperidine-1-carboxylic acid benzyl ester in 400 mL THF and 300 mL methanol was hydrogenated at room temperature over 3.5 g Pd/C (10%). The mixture was filtered and evaporated to dryness to yield the crude title compound which was used without further purification in the consecutive step. MS(m/e): 207.1 (MH⁺).

### Intermediate 9

### 2-(3,4-Dichloro-phenyl)-4-[4-(ethyl-methanesulfonyl-amino)-piperidin-1-yl]-2-methyl-butyric acid

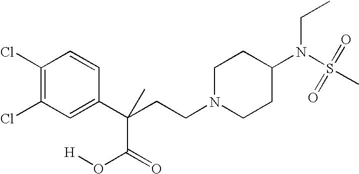

### a) Step 1:

### 2-(3,4-Dichloro-phenyl)-4-[4-(ethyl-methanesulfonyl-amino)-piperidin-1-yl]-2-methyl-butyric acid methyl ester

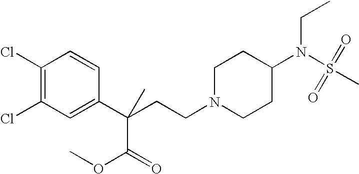

In analogy to the procedure described for the synthesis of 2-(3,4-dichloro-phenyl)-4-(4-methanesulfonylamino-piperidin-1-yl)-2-methyl-butyric acid methyl ester (intermediate 1, step 4) the title compound was prepared from 4-bromo-2-(3,4-dichloro-phenyl)-2-methyl-butyric acid methyl ester and N-ethyl-N-piperidin-4-yl-methanesulfonamide (intermediate 8) as light yellow viscous oil. MS(m/e): 467.0 (MH⁺).

### b) Step 2:

In analogy to the procedure described for the synthesis of 2-(3,4-dichloro-phenyl)-4-(4-methanesulfonylamino-piperidin-1-yl)-2-methyl-butyric acid (intermediate 1, step 5) the title compound was prepared from 2-(3,4-dichloro-phenyl)-4-[4-(ethyl-methanesulfonyl-amino)-piperidin-1-yl]-2-methyl-butyric acid methyl ester by saponification with LiOH.H₂O and conversion with HCl into the respective salt as white foam. MS(m/e): 451.0 (MH⁺).

### Intermediate 10

### 2-(3,4-Dichloro-phenyl)-2-methyl-4-[4-(2-oxo-pyrrolidin-1-yl)-piperidin-1-yl]-butyric acid

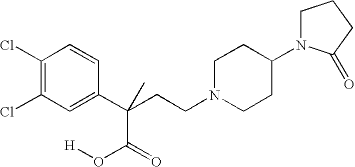

### a) Step 1:

### 2-(3,4-Dichloro-phenyl)-2-methyl-4-[4-(2-oxo-pyrrolidin-1-yl)-piperidin-1-yl]-butyric acid methyl ester

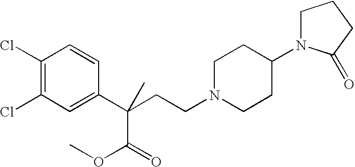

In analogy to the procedure described for the synthesis of 2-(3,4-dichloro-phenyl)-4-(4-methanesulfonylamino-piperidin-1-yl)-2-methyl-butyric acid methyl ester (intermediate 1, step 4) the title compound was prepared from 4-bromo-2-(3,4-dichloro-phenyl)-2-methyl-butyric acid methyl ester and 1-piperidin-4-yl-pyrrolidin-2-one ( commercially available) as light yellow viscous oil. MS(m/e): 427.1 (MH⁺).

### b) Step 2:

In analogy to the procedure described for the synthesis of 2-(3,4-dichloro-phenyl)-4-(4-methanesulfonylamino-piperidin-1-yl)-2-methyl-butyric acid (intermediate 1, step 5) the title compound was prepared from 2-(3,4-dichloro-phenyl)-2-methyl-4-[4-(2-oxo-pyrrolidin-1-yl)-piperidin-1-yl]-butyric acid methyl ester by saponification with LiOH.H₂O and conversion with HCl into the respective salt as white foam. MS(m/e): 415.2 (MH⁺).

### Intermediate 11

### 4-[4-(Acetyl-methyl-amino)-piperidin-1-yl]-2-(3,4-dichloro-phenyl)-2-methyl-butyric acid

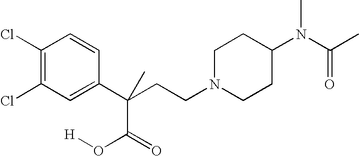

### a) Step 1:

### 4-[4-(Acetyl-methyl-amino)-piperidin-1-yl]-2-(3,4-dichloro-phenyl)-2-methyl-butyric acid methyl ester

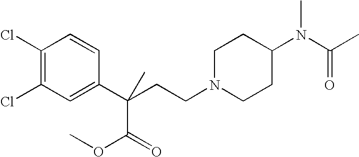

In analogy to the procedure described for the synthesis of 2-(3,4-dichloro-phenyl)-4-(4-methanesulfonylamino-piperidin-1-yl)-2-methyl-butyric acid methyl ester (intermediate 1, step 4) the title compound was prepared from 4-bromo-2-(3,4-dichloro-phenyl)-2-methyl-butyric acid methyl ester and N-methyl-N-piperidin-4-yl-acetamide (WO2005019194) as yellow viscous oil. MS(m/e): 415.3 (MH⁺).

### b) Step 2:

In analogy to the procedure described for the synthesis of 2-(3,4-dichloro-phenyl)-4-(4-methanesulfonylamino-piperidin-1-yl)-2-methyl-butyric acid (intermediate 1, step 5) the title compound was prepared from 4-[4-(acetyl-methyl-amino)-piperidin-1-yl]-2-(3,4-dichloro-phenyl)-2-methyl-butyric acid methyl ester by saponification with LiOH.H₂O and conversion with HCR into the respective salt as white foam. MS(m/e): 401.1 (MH⁺).

### Intermediate 12

### (3-Fluoro-4-trifluoromethyl-benzyl)-methyl-amine, hydrochloride

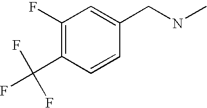

A mixture of 7.65 g (39.8 mmol) 3-fluoro-4-trifluoro-benzaldehyde, 24.39 mL (49.0 mmol) methylamine (2M in THF), 3.60 mL acetic acid and 12.46 g (59.0 mmol) sodium triacetoxyborohydride in 200 mL THF was stirred for 22 h at room temperature. Water and Na₂CO₃ aq. was added and the mixture was extracted with ethyl acetate. The organic phases were washed with NaCl aq., dried with Na₂SO₄, filtered and evaporated to dryness. The residue was purified by flash column chromatography on silica eluting with a gradient formed from DCM, methanol and NH₃ aq. The combined product containing fractions were evaporated and treated with HCl in diethyl ether. The precipitate was filtered, washed with diethyl ether and dried to yield 4.00 g (42%) of the title compound as white solid. MS(m/e): 208.1 (MH⁺).

### Intermediate 13

### (4-Fluoro-3-trifluoromethyl-benzyl)-methyl-amine, hyrdochloride

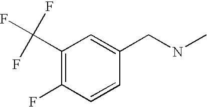

In analogy to the procedure described for the synthesis of (3-fluoro-4-trifluoromethyl-benzyl)-methyl-amine, hydrochloride (intermediate 12) the title compound was prepared from 4-fluoro-3-trifluoro-benzaldehyde and methylamine under reductive conditions as white solid. MS(m/e): 208.3 (MH⁺).

### Intermediate 14

### 4-[4-(Acetyl-cyclopropyl-amino)-piperidin-1-yl]-2-(3,4-dichloro-phenyl)-2-methyl-butyric acid

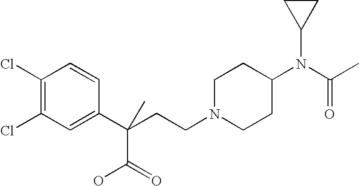

In analogy to the procedure described for the synthesis of 2-(3,4-dichloro-phenyl)-4-(4-methanesulfonylamino-piperidin-1-yl)-butyric acid (intermediate 2) the title compound was prepared from 2-(3,4-dichloro-phenyl)-4-oxo-butyric acid methyl ester, N-Cyclopropyl-N-piperidin-4-yl-acetamide (intermediate 22) and subsequent saponification as exemplified in the synthesis of intermediate 2, c) step 3. MS(m/e): 427.2 (MH⁺).

### Intermediate 15

### 4-{4-[Acetyl-(2-methoxy-ethyl)-amino]-piperidin-1-yl} -2-(3,4-dichloro-phenyl)-2-methyl-butyric acid

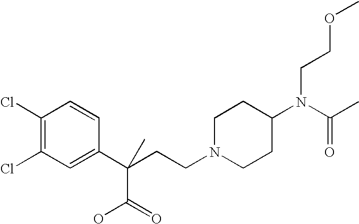

In analogy to the procedure described for the synthesis of 2-(3,4-dichloro-phenyl)-4-(4-methanesulfonylamino-piperidin-1-yl)-butyric acid (intermediate 2) the title compound was prepared from 2-(3,4-dichloro-phenyl)-4-oxo-butyric acid methyl ester, N-(2-Methoxy-ethyl)-N-piperidin-4-yl-acetamide, hydrochloride (intermediate 23) and subsequent saponification as exemplified in the synthesis of intermediate 2, c) step 3. MS(m/e): 445.1 (MH⁺).

### Intermediate 16

### 4-(4-Acetylamino-piperidin-1-yl)-2-(3,4-dichloro-phenyl)-2-methylbutyric acid

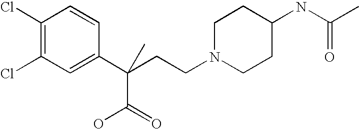

In analogy to the procedure described for the synthesis of 2-(3,4-Dichloro-phenyl)-4-(4-methanesulfonylamino-piperidin-1-yl)-butyric acid (intermediate 2) the title compound was prepared from 2-(3,4-Dichloro-phenyl)-4-oxo-butyric acid methyl ester, 4-Acetylamino-piperidine (commercially available) and subsequent saponification as exemplified in the synthesis of intermediate 2, c) step 3. MS(m/e): 387.2 (MH⁺).

### Intermediate 17

### 4-[4-(Acetyl-propyl-amino)-piperidin-1-yl]-2-(3,4-dichloro-phenyl)-2-methyl-butyric acid

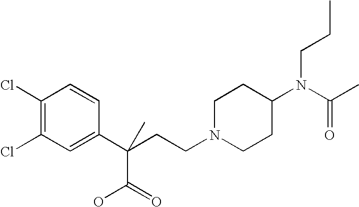

In analogy to the procedure described for the synthesis of 2-(3,4-Dichloro-phenyl)-4-(4-methanesulfonylamino-piperidin-1-yl)-butyric acid (intermediate 2) the title compound was prepared from 2-(3,4-Dichloro-phenyl)-4-oxo-butyric acid methyl ester, N-Piperidin-4-yl-N-propyl-acetamide (WO 9410146) and subsequent saponification as exemplified in the synthesis of intermediate 2, c) step 3. MS(m/e): 429.2 (MH⁺).

### Intermediate 18

### 4-[4-(Acetyl-ethyl-amino)-piperidin-1-yl]-2-(3,4-dichloro-phenyl)-2-methyl-butyric acid

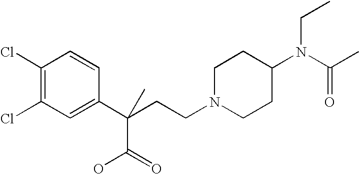

In analogy to the procedure described for the synthesis of 2-(3,4-Dichloro-phenyl)-4-(4-methanesulfonylamino-piperidin-1-yl)-butyric acid (intermediate 2) the title compound was prepared from 2-(3,4-Dichloro-phenyl)-4-oxo-butyric acid methyl ester, N-Ethyl-N-piperidin-4-yl-acetamide (EP 457686) and subsequent saponification as exemplified in the synthesis of intermediate 2, c) step 3. MS(m/e): 415.2 (MH⁺).

### Intermediate 19

### 1-(4-Fluoro-3-trifluoromethyl-phenyl)-ethylamine; hydrochloride

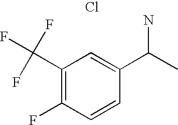

A mixture of log (49 mmol) 4-Fluoro-3-(trifluoromethyl) acetopheneone (commercially available), 27.5 g (97 mmol) titanium tetraisopropoxide and 34 mL (243 mmol) 7 N ammonia in MeOH at 0° C. was stirred over the weekend at 20° C. Subsequently, 5.5 g (146 mmol) sodium borohydride was added and the mixture allowed to come from 0° C. to room temperature over night. Water and ammonia was added and the mixture was filtered through decalit and washed with ethyl acetate. The aqueous phase was separated washed with ethyl acetate and the combined organic phases washed with NaCL sat. aq., dried with Na₂SO₄ and evaporated to dryness. The residue was purified by column chromatography eluting with a gradient formed from DCM, methanol and ammonia. The product containing fractions were evaporated to dryness and transformed with HCl in diethyl ether to the respective hydrochloride salt. Yield: 4.4 g ( 37%). MS(m/e): 207.9 (MH⁺).

### Intermediate 20

### 1-(4-Fluoro-3-trifluoromethyl-phenyl)-ethyl-methyl amine; hydrochloride

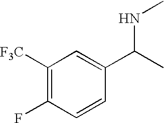

In analogy to the procedure described for the synthesis of Intermediate 20, 1-(4-Fluoro-3-trifluoromethyl-phenyl)-ethylamine; hydrochloride the title compound was prepared from 4-fluoro-3-(trifluoromethyl) acetopheneone (commercially available) and methylamine. MS(m/e): 191.2/222.1 (MH⁺).

### Intermediate 21

### N-Cyclopropyl-N-piperidin-4-yl-acetamide

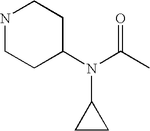

A mixture of 6.25 g (27 mmol) 1-(benzyloxycarbonyl)-4-piperidinone (commercially available, 1.83 g (32 mmol) cyclopropyl amine, 2.3 mL acetic acid and 8.51 g (40 mmol) sodium triacetoxyborohydride was stirred from 0° C. to room temperature over night. Water and Na₂CO₃ aq. was added and the mixture extracted with ethyl acetate. The organic phase was washed with NaCl sat. aq. , dried with Na₂SO₄ and evaporated to dryness. The residue was taken up in 100 mL DCM and 4.48 mL (32 mmol) NEt₃ and 2.09 mL acetyl chloride was added and allowed to stirr at 0° C. for 2 h. The mixture was evaporated and isolute was added and the residue was purified by column chromatography on silica eluting with a gradient formed from DCM, methanol and ammonia. The product containing fractions were evaporated and the residue taken up in methanol and hydrogenated over Pd/C with H₂. Filtration and evaporation yielded 4.5 g (92%) of the title compound as light yellow solid. MS(m/e): 183.2 (MH⁺).

### Intermediate 22

### N-(2-Methoxy-ethyl)-N-piperidin-4-yl-acetamide, hydrochloride

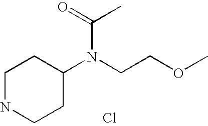

In analogy to the procedure described for the synthesis of N-cyclopropyl-N-piperidin-4-yl-acetamide (intermediate 22) the title compound was prepared from 1-(benzyloxycarbonyl)-4-piperidinone (commercially available and 2-methoxyethyloamine.

The title compound was obtained as hydrochloride salt by treatment of the free base with HCl. MS(m/e): 201.1 (MH⁺).

### Example 1

### 2-(3,4-Dichloro-phenyl)-N-(3-fluoro-4-methoxy-benzyl)-4-(4-methanesulfonylamino-piperidin-1-yl)-2,N-dimethyl-butyramide

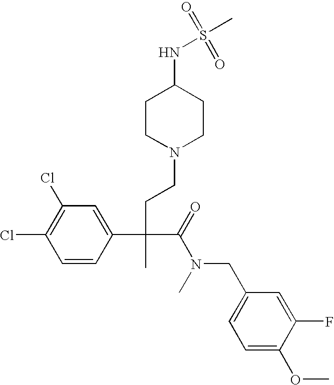

A mixture of 30 mg crude 2-(3,4-dichloro-phenyl)-4-(4-methanesulfonylamino-piperidin-1-yl)-2-methyl-butyric acid, 14 mg (0.068 mmol) (3-fluoro-4-methoxy-benzyl)-methyl-amine, hydrochloride, 0.33 mL TBTU/DMF (0.2N) and 50 uL DIPEA in 1.2 mL DMF was stirred at room temperature for 16 h. The mixture was subjected to purification by preparative HPLC on reversed phase eluting with a gradient formed from acetonitrile, water and NEt₃. The product containing fractions were evaporated to yield 6.5 mg of the title compound. MS(m/e): 574.3 (MH⁺).

In analogy to the procedure described for the synthesis of 2-(3,4-dichloro-phenyl)-N-(3-fluoro-4-methoxy-benzyl)-4-(4-methanesulfonylamino-piperidin-1-yl)-2,N-dimethyl-butyramide (example 1) further piperidine derivatives have been prepared from the starting materials listed in table 1. Optionally diastereoisomeric mixture have been separated by column chromatography. Table 1 comprises example 2-example 110.

TABLE 1

| | | | | | MW | hNK3 |
|---|---|---|---|---|---|---|
| | | | | | found | Ki |
| No | structure | MW | Systematic Name | starting materials | (MH+) | (uM) |
| 1 | 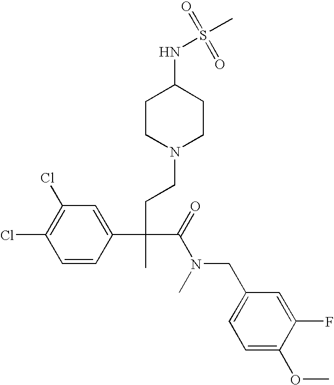 | 574.5 | 2-(3,4-Dichloro-phenyl)-N-(3-fluoro-4-methoxy-benzyl)-4-(4-methanesulfonylami-no-piperidin-1-yl)-2,N-dimethyl-butyramide | 2-(3,4-Dichloro-phenyl)-4-(4-methanesulfonylamino-piperidin-1-yl)-2-methyl-butyricacid (intermediate1) and (3-Fluoro-4-methoxy-benzyl)-methyl-amine(commerciallyavailable | 574.3 | 0.1872 |
| 2 | 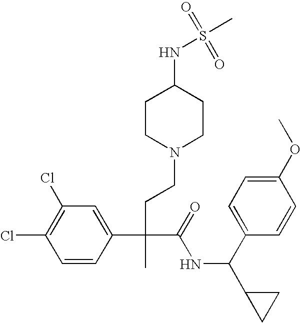 | 582.6 | N-[Cyclopropyl-(4-methoxy-phenyl)-methyl]-2-(3,4-dichloro-phenyl)-4-(4-methanesulfonylamino-piperidin-1-yl)-2-methyl-butyramide | 2-(3,4-Dichloro-phenyl)-4-(4-methanesulfonylami-no-piperidin-1-yl)-2-methyl-butyricacid (intermediate1) and C-Cyclopropyl-C-(4-methoxy-phenyl)-methylamine(commerciallyavailable) | 584.3 | 0.7035 |
| 3 | 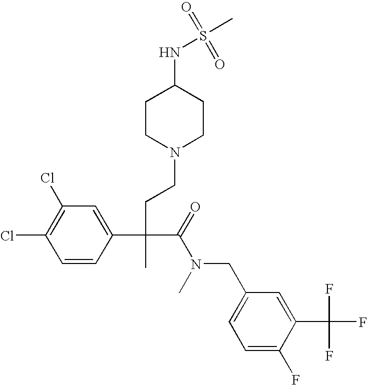 | 612.5 | 2-(3,4-Dichloro-phenyl)-N-(4-fluoro-3-trifluoromethyl-benzyl)-4-(4-methanesulfonylamino-piperidin-1-yl)-2,N-dimethyl-butyramide | 2-(3,4-Dichloro-phenyl)-4-(4-methanesulfonylami-no-piperidin-1-yl)-2-methyl-butyricacid (intermediate1) and (4-Fluoro-3-trifluoromethyl-benzyl)-methyl-amine,hyrdochloride(intermediate 13) | 612.1 | 0.0232 |
| 4 | 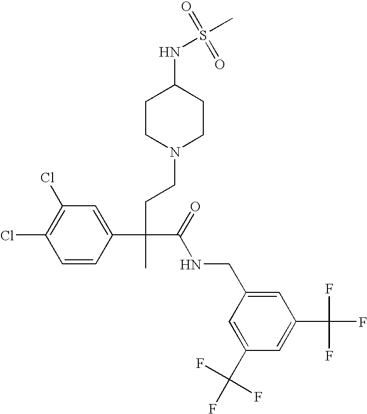 | 648.5 | N-(3,5-Bis-trifluoromethyl-benzyl)-2-(3,4-dichloro-phenyl)-4-(4-methanesulfonylamino-piperidin-1-yl)-2-methyl-butyramide | 2-(3,4-Dichloro-phenyl)-4-(4-methanesulfonylami-no-piperidin-1-yl)-2-methyl-butyricacid (intermediate1) and 3,5-Bis-trifluoromethyl-benzylamine(commerciallyavailable) | 648.3 | 0.1396 |
| 5 | 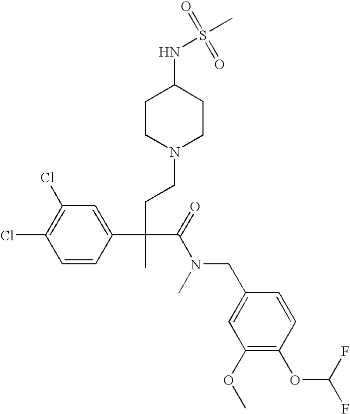 | 622.6 | 2-(3,4-Dichloro-phenyl)-N-(4-difluoromethoxy-3-methoxy-benzyl)-4-(4-methanesulfonylamino-piperidin-1-yl)-2,N-dimethyl-butyramide | 2-(3,4-Dichloro-phenyl)-4-(4-methanesulfonylamino-piperidin-1-yl)-2-methyl-butyricacid (intermediate1) and (4-Difluoromethoxy-3-methoxy-benzyl)-methyl-amine(commerciallyavailable) | 622.2 | 0.2716 |
| 6 | 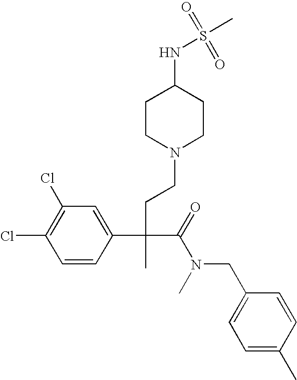 | 540.6 | 2-(3,4-Dichloro-phenyl)-4-(4-methanesulfonylamino-piperidin-1-yl)-2,N-dimethyl-N-(4-methyl-benzyl)-butyramide | 2-(3,4-Dichloro-phenyl)-4-(4-methanesulfonylami-no-piperidin-1-yl)-2-methyl-butyricacid (intermediate1) and Methyl-(4-methyl-benzyl)-amine(commerciallyavailable) | 540.4 | 0.0774 |
| 7 | 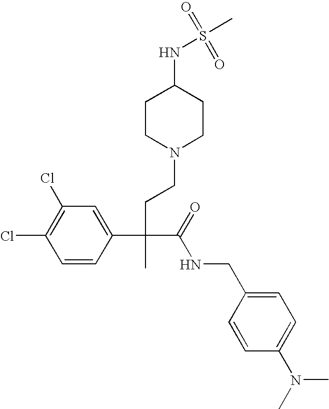 | 555.6 | 2-(3,4-Dichloro-phenyl)-N-(4-dimethylamino-benzyl)-4-(4-methanesulfonylami-no-piperidin-1-yl)-2-methyl-butyramide | 2-(3,4-Dichloro-phenyl)-4-(4-methanesulfonylami-no-piperidin-1-yl)-2-methyl-butyricacid (intermediate1) and (4-Aminomethyl-phenyl)-dimethyl-amine(commerciallyavailable) | 555.3 | 0.6205 |
| 8 | 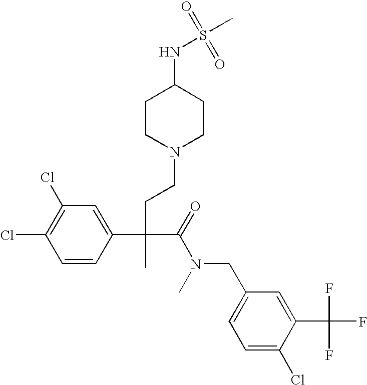 | 629.0 | N-(4-Chloro-3-trifluoromethyl-benzyl)-2-(3,4-dichloro-phenyl)-4-(4-methanesulfonylamino-piperidin-1-yl)-2,N-dimethyl-butyramide | 2-(3,4-Dichloro-phenyl)-4-(4-methanesulfonylami-no-piperidin-1-yl)-2-methyl-butyricacid (intermediate1) and (4-Chloro-3-trifluoromethyl-benzyl)-methyl-amine(commerciallyavailable) | 630.3 | 0.0226 |
| 9 | 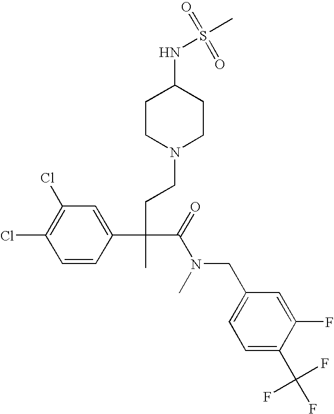 | 612.5 | 2-(3,4-Dichloro-phenyl)-N-(3-fluoro-4-trifluoromethyl-benzyl)-4-(4-methanesulfonylamino-piperidin-1-yl)-2,N-dimethyl-butyramide | 2-(3,4-Dichloro-phenyl)-4-(4-methanesulfonylami-no-piperidin-1-yl)-2-methyl-butyricacid (intermediate1) and (3-Fluoro-4-trifluoromethyl-benzyl)-methyl-amine,hydrochloride(intermediate 12) | 612.3 | 0.03 |
| 10 | 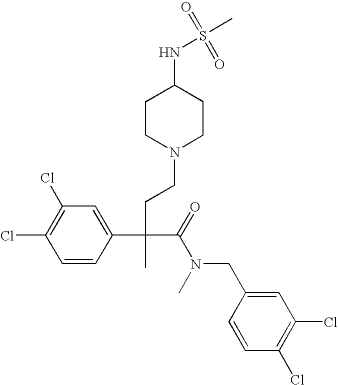 | 595.4 | N-(3,4-Dichloro-benzyl)-2-(3,4-dichloro-phenyl)-4-(4-methanesulfonylamino-piperidin-1-yl)-2,N-dimethyl-butyramide | 2-(3,4-Dichloro-phenyl)-4-(4-methanesulfonylami-no-piperidin-1-yl)-2-methyl-butyricacid (intermediate1) and (3,4-Dichloro-benzyl)-methyl-amine(commerciallyavailable) | 596.1 | 0.024 |
| 11 | 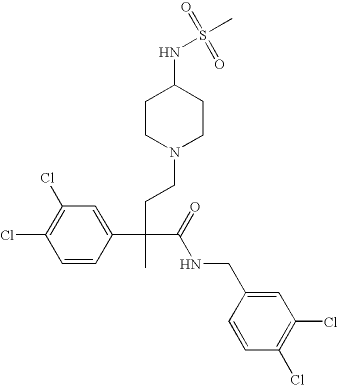 | 581.4 | N-(3,4-Dichloro-benzyl)-2-(3,4-dichloro-phenyl)-4-(4-methanesulfonylamino-piperidin-1-yl)-2-methyl-butyramide | 2-(3,4-Dichloro-phenyl)-4-(4-methanesulfonylami-no-piperidin-1-yl)-2-methyl-butyricacid (intermediate1) and 3,4-Dichloro-benzylamine(commerciallyavailable) | 582 | 0.0442 |
| 12 | 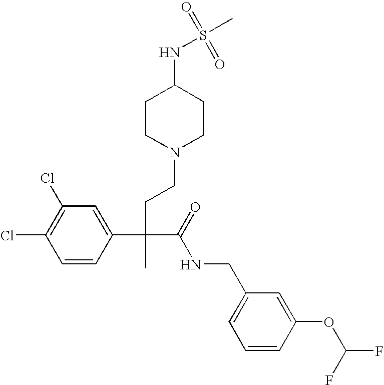 | 578.5 | 2-(3,4-Dichloro-phenyl)-N-(3-difluoromethoxy-benzyl)-4-(4-methanesulfonylamino-piperidin-1-yl)-2-methyl-butyramide | 2-(3,4-Dichloro-phenyl)-4-(4-methanesulfonylami-no-piperidin-1-yl)-2-methyl-butyricacid (intermediate1) and 3-Difluoromethoxy-benzylamine(commerciallyavailable) | 578.1 | 0.3094 |
| 13 | 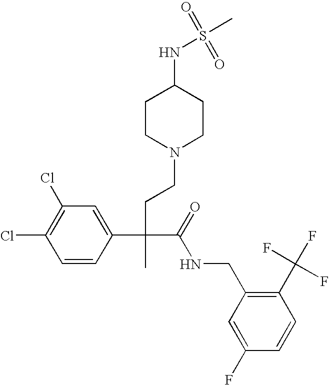 | 598.5 | 2-(3,4-Dichloro-phenyl)-N-(5-fluoro-2-trifluoromethyl-benzyl)-4-(4-methanesulfonylamino-piperidin-1-yl)-2-methyl-butyramide | 2-(3,4-Dichloro-phenyl)-4-(4-methanesulfonylami-no-piperidin-1-yl)-2-methyl-butyricacid (intermediate1) and 5-Fluoro-2-trifluoromethyl-benzylamine(commerciallyavailable) | 598.1 | 0.5804 |
| 14 | 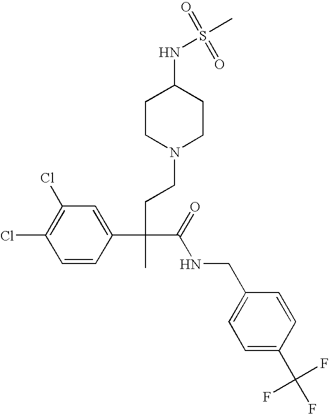 | 580.5 | 2-(3,4-Dichloro-phenyl)-4-(4-methanesulfonylamino-piperidin-1-yl)-2-methyl-N-(4-trifluoromethyl-benzyl)-butyramide | 2-(3,4-Dichloro-phenyl)-4-(4-methanesulfonylami-no-piperidin-1-yl)-2-methyl-butyricacid (intermediate1) and 4-Trifluoromethyl-benzylamine(commerciallyavailable) | 580 | 0.058 |
| 15 | 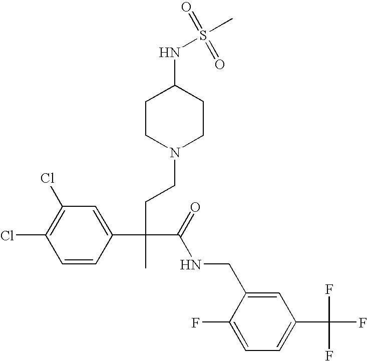 | 598.5 | 2-(3,4-Dichloro-phenyl)-N-(2-fluoro-5-trifluoromethyl-benzyl)-4-(4-methanesulfonylamino-piperidin-1-yl)-2-methyl-butyramide | 2-(3,4-Dichloro-phenyl)-4-(4-methanesulfonylami-no-piperidin-1-yl)-2-methyl-butyricacid (intermediate1) and 2-Fluoro-5-trifluoromethyl-benzylamine(commerciallyavailable) | 598.3 | 0.1661 |
| 16 | 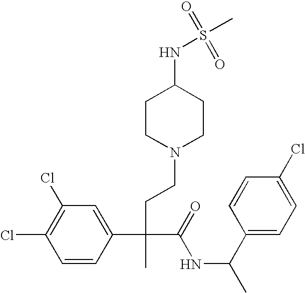 | 561.0 | N-[1-(4-Chloro-phenyl)-ethyl]-2-(3,4-dichloro-phenyl)-4-(4-methanesulfonylamino-piperidin-1-yl)-2-methyl-butyramide | 2-(3,4-Dichloro-phenyl)-4-(4-methanesulfonylami-no-piperidin-1-yl)-2-methyl-butyricacid (intermediate1) and 1-(4-Chloro-phenyl)-ethylamine(commerciallyavailable) | 562.5 | 0.1735 |
| 17 | 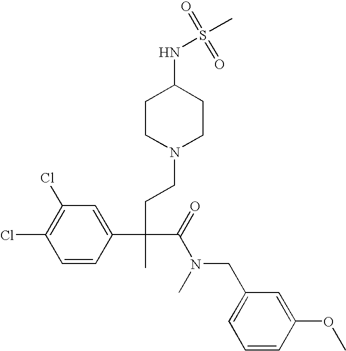 | 556.6 | 2-(3,4-Dichloro-phenyl)-4-(4-methanesulfonylamino-piperidin-1-yl)-N-(3-methoxy-benzyl)-2,N-dimethyl-butyramide | 2-(3,4-Dichloro-phenyl)-4-(4-methanesulfonylami-no-piperidin-1-yl)-2-methyl-butyricacid (intermediate1) and (3-Methoxy-benzyl)-methyl-amine(commerciallyavailable) | 556.1 | 0.2127 |
| 18 | 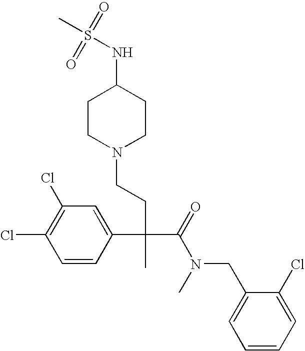 | 561.0 | N-(2-Chloro-benzyl)-2-(3,4-dichloro-phenyl)-4-(4-methanesulfonylamino-piperidin-1-yl)-2,N-dimethyl-butyramide | 2-(3,4-Dichloro-phenyl)-4-(4-methanesulfonylami-no-piperidin-1-yl)-2-methyl-butyricacid (intermediate1) and (2-Chloro-benzyl)-methyl-amine(commerciallyavailable) | 560.2 | 0.0333 |
| 19 | 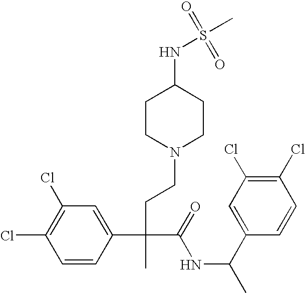 | 595.4 | 2-(3,4-Dichloro-phenyl)-N-[1-(3,4-dichloro-phenyl)-ethyl]-4-(4-methanesulfonylamino-piperidin-1-yl)-2-methyl-butyramide | 2-(3,4-Dichloro-phenyl)-4-(4-methanesulfonylami-no-piperidin-1-yl)-2-methyl-butyricacid (intermediate1) and 1-(3,4-Dichloro-phenyl)-ethylamine(commerciallyavailable) | 596.3 | 0.0599 |
| 20 | 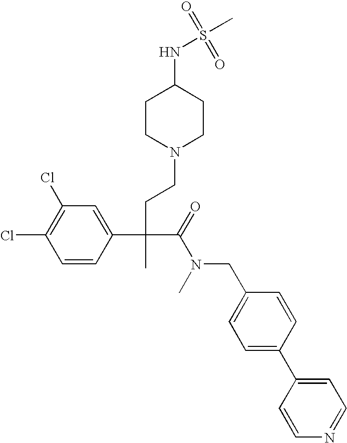 | 603.6 | 2-(3,4-Dichloro-phenyl)-4-(4-methanesulfonylamino-piperidin-1-yl)-2,N-dimethyl-N-(4-pyridin-4-yl-benzyl)-butyramide | 2-(3,4-Dichloro-phenyl)-4-(4-methanesulfonylami-no-piperidin-1-yl)-2-methyl-butyricacid (intermediate1) and Methyl-(4-pyridin-4-yl-benzyl)-amine(commerciallyavailable) | 603.3 | 0.2032 |
| 21 | 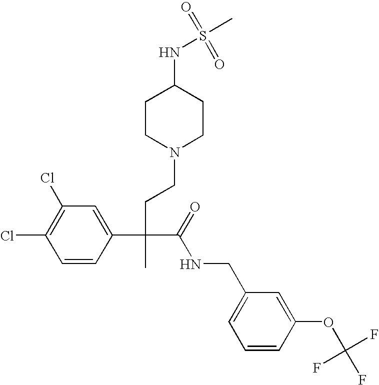 | 596.5 | 2-(3,4-Dichloro-phenyl)-4-(4-methanesulfonylamino-piperidin-1-yl)-2-methyl-N-(3-trifluoromethoxy-benzyl)-butyramide | 2-(3,4-Dichloro-phenyl)-4-(4-methanesulfonylami-no-piperidin-1-yl)-2-methyl-butyricacid (intermediate1) and 3-Trifluoromethoxy-benzylamine(commerciallyavailable) | 596.2 | 0.1468 |
| 22 | 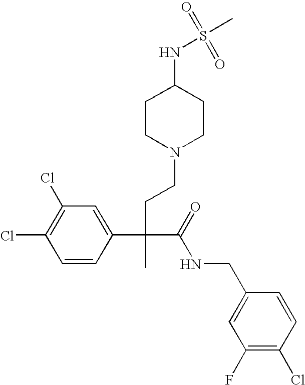 | 564.9 | N-(4-Chloro-3-fluoro-benzyl)-2-(3,4-dichloro-phenyl)-4-(4-methanesulfonylamino-piperidin-1-yl)-2-methyl-butyramide | 2-(3,4-Dichloro-phenyl)-4-(4-methanesulfonylami-no-piperidin-1-yl)-2-methyl-butyricacid (intermediate1) and 4-Chloro-3-fluoro-benzylamine(commerciallyavailable) | 566.3 | 0.0708 |
| 23 | 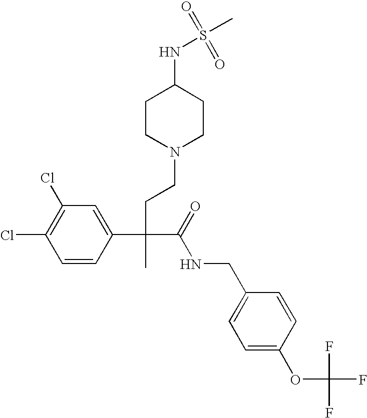 | 596.5 | 2-(3,4-Dichloro-phenyl)-4-(4-methanesulfonylamino-piperidin-1-yl)-2-methyl-N-(4-trifluoromethoxy-benzyl)-butyramide | 2-(3,4-Dichloro-phenyl)-4-(4-methanesulfonylami-no-piperidin-1-yl)-2-methyl-butyricacid (intermediate1) and 4-Trifluoromethoxy-benzylamine(commerciallyavailable) | 596.2 | 0.1403 |
| 24 | 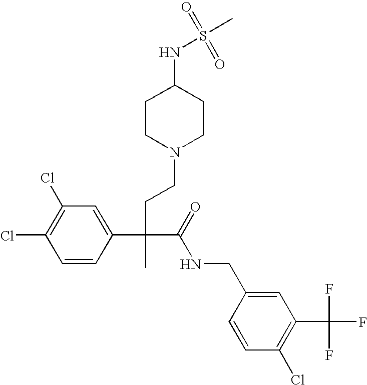 | 614.9 | N-(4-Chloro-3-trifluoromethyl-benzyl)-2-(3,4-dichloro-phenyl)-4-(4-methanesulfonylamino-piperidin-1-yl)-2-methyl-butyramide | 2-(3,4-Dichloro-phenyl)-4-(4-methanesulfonylami-no-piperidin-1-yl)-2-methyl-butyricacid (intermediate1) and 4-Chloro-3-trifluoromethyl-benzylamine(commerciallyavailable) | 616.3 | 0.0714 |
| 25 | 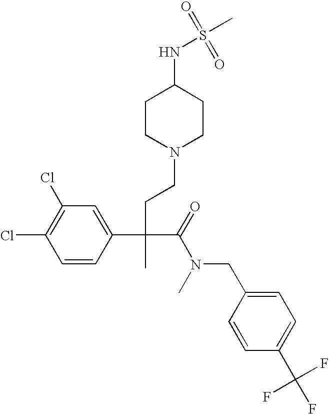 | 594.5 | 2-(3,4-Dichloro-phenyl)-4-(4-methanesulfonylamino-piperidin-1-yl)-2,N-dimethyl-N-(4-trifluoromethyl-benzyl)-butyramide | 2-(3,4-Dichloro-phenyl)-4-(4-methanesulfonylami-no-piperidin-1-yl)-2-methyl-butyricacid (intermediate1) and Methyl-(4-trifluoromethyl-benzyl)-amine(commerciallyavailable | 594.2 | 0.0287 |
| 26 | 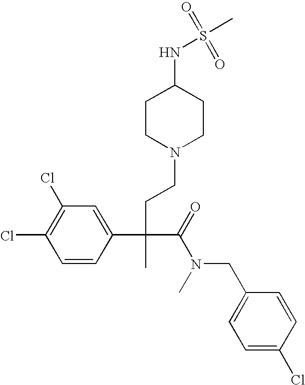 | 561.0 | N-(4-Chloro-benzyl)-2-(3,4-dichloro-phenyl)-4-(4-methanesulfonylamino-piperidin-1-yl)-2,N-dimethyl-butyramide | 2-(3,4-Dichloro-phenyl)-4-(4-methanesulfonylami-no-piperidin-1-yl)-2-methyl-butyricacid (intermediate1) and (4-Chloro-benzyl)-methyl-amine(commerciallyavailable) | 560.3 | 0.0265 |
| 27 | 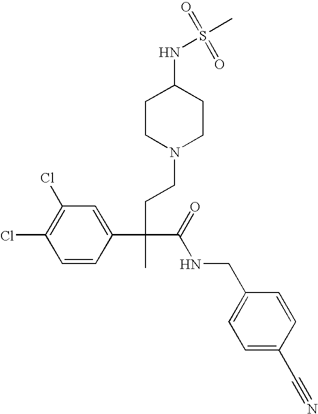 | 537.5 | N-(4-Cyano-benzyl)-2-(3,4-dichloro-phenyl)-4-(4-methanesulfonylamino-piperidin-1-yl)-2-methyl-butyramide | 2-(3,4-Dichloro-phenyl)-4-(4-methanesulfonylami-no-piperidin-1-yl)-2-methyl-butyricacid (intermediate1) and 4-Aminomethyl-benzonitrile(commerciallyavailable) | 537.3 | 0.2633 |
| 28 | 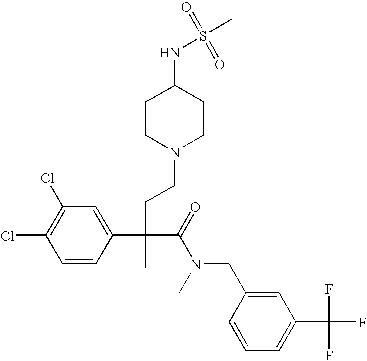 | 594.5 | 2-(3,4-Dichloro-phenyl)-4-(4-methanesulfonylamino-piperidin-1-yl)-2,N-dimethyl-N-(3-trifluoromethyl-benzyl)-butyramide | 2-(3,4-Dichloro-phenyl)-4-(4-methanesulfonylami-no-piperidin-1-yl)-2-methyl-butyricacid (intermediate1) and Methyl-(3-trifluoromethyl-benzyl)-amine(commerciallyavailable) | 594.2 | 0.0217 |
| 29 | 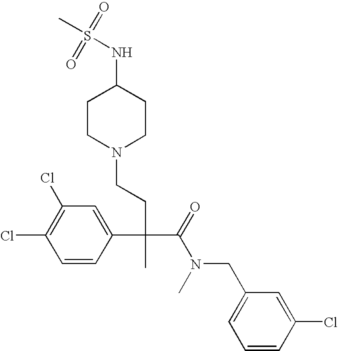 | 561.0 | N-(3-Chloro-benzyl)-2-(3,4-dichloro-phenyl)-4-(4-methanesulfonylamino-piperidin-1-yl)-2,N-dimethyl-butyramide | 2-(3,4-Dichloro-phenyl)-4-(4-methanesulfonylami-no-piperidin-1-yl)-2-methyl-butyricacid (intermediate1) and (3-Chloro-benzyl)-methyl-amine(commerciallyavailable) | 560.5 | 0.0485 |
| 30 | 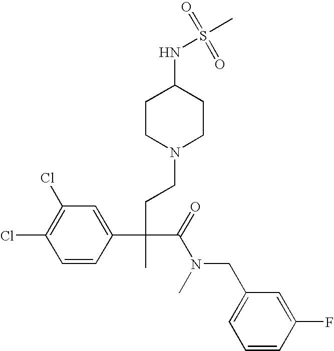 | 544.5 | 2-(3,4-Dichloro-phenyl)-N-(3-fluoro-benzyl)-4-(4-methanesulfonylamino-piperidin-1-yl)-2,N-dimethyl-butyramide | 2-(3,4-Dichloro-phenyl)-4-(4-methanesulfonylami-no-piperidin-1-yl)-2-methyl-butyricacid (intermediate1) and (3-Fluoro-benzyl)-methyl-amine(commerciallyavailable) | 544.2 | 0.1233 |
| 31 | 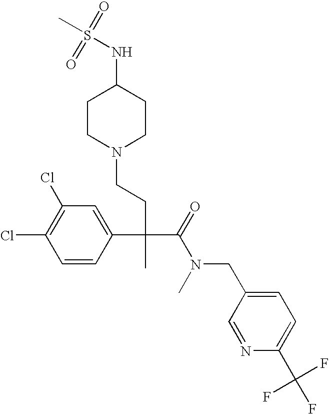 | 595.5 | 2-(3,4-Dichloro-phenyl)-4-(4-methanesulfonylamino-piperidin-1-yl)-2,N-dimethyl-N-(6-trifluoromethyl-pyridin-3-ylmethyl)-butyramide | 2-(3,4-Dichloro-phenyl)-4-(4-methanesulfonylami-no-piperidin-1-yl)-2-methyl-butyricacid (intermediate1) and Methyl-(6-trifluoromethyl-pyridin-3-ylmethyl)-amine(commerciallyavailable) | 595.2 | 0.5004 |
| 32 | 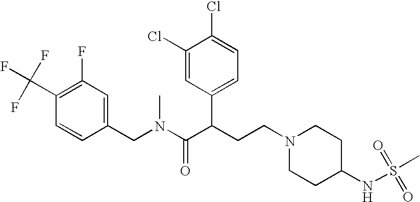 | 598.5 | 2-(3,4-Dichloro-phenyl)-N-(3-fluoro-4-trifluoromethyl-benzyl)-4-(4-methanesulfonylamino-piperidin-1-yl)-N-methyl-butyramide | 2-(3,4-Dichloro-phenyl)-4-(4-methanesulfonylami-no-piperidin-1-yl)-butyric acid(intermediate 2)and (3-Fluoro-4-trifluoromethyl-benzyl)-methyl-amine,hydrochloride(intermediate 12) | 598.3 | 0.0617 |
| 33 | 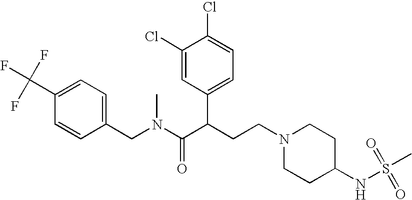 | 580.5 | 2-(3,4-Dichloro-phenyl)-4-(4-methanesulfonylamino-piperidin-1-yl)-N-methyl-N-(4-trifluoromethyl-benzyl)-butyramide | 2-(3,4-Dichloro-phenyl)-4-(4-methanesulfonylami-no-piperidin-1-yl)-butyric acid(intermediate 2)and Methyl-(4-trifluoromethyl-benzyl)-amine(commerciallyavailable) | 580.3 | 0.0816 |
| 34 | 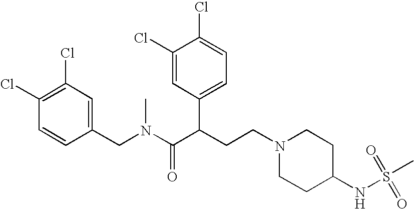 | 581.4 | N-(3,4-Dichloro-benzyl)-2-(3,4-dichloro-phenyl)-4-(4-methanesulfonylamino-piperidin-1-yl)-N-methyl-butyramide | 2-(3,4-Dichloro-phenyl)-4-(4-methanesulfonylami-no-piperidin-1-yl)-butyric acid(intermediate 2)and (3,4-Dichloro-benzyl)-methyl-amine(commerciallyavailable) | 582.1 | 0.0775 |
| 35 | 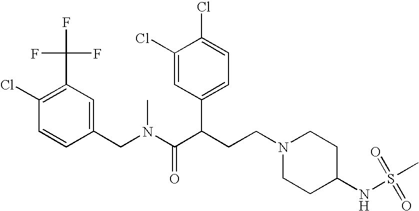 | 614.9 | N-(4-Chloro-3-trifluoromethyl-benzyl)-2-(3,4-dichloro-phenyl)-4-(4-methanesulfonylamino-piperidin-1-yl)-N-methyl-butyramide | 2-(3,4-Dichloro-phenyl)-4-(4-methanesulfonylami-no-piperidin-1-yl)-butyric acid(intermediate 2)and (4-Chloro-3-trifluoromethyl-benzyl)-methyl-amine(commerciallyavailable) | 616.1 | 0.1494 |
| 36 | 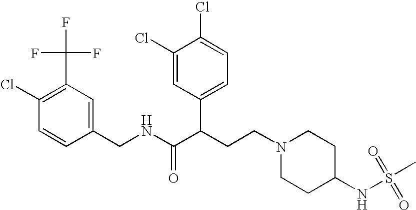 | 600.9 | N-(4-Chloro-3-trifluoromethyl-benzyl)-2-(3,4-dichloro-phenyl)-4-(4-methanesulfonylamino-piperidin-1-yl)-butyramide | 2-(3,4-Dichloro-phenyl)-4-(4-methanesulfonylami-no-piperidin-1-yl)-butyric acid(intermediate 2)and 4-Chloro-3-trifluoromethyl-benzylamine(commerciallyavailable) | 600.2 | 0.3541 |
| 37 | 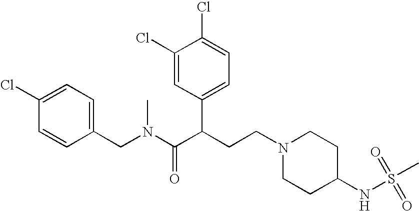 | 546.9 | N-(4-Chloro-benzyl)-2-(3,4-dichloro-phenyl)-4-(4-methanesulfonylamino-piperidin-1-yl)-N-methyl-butyramide | 2-(3,4-Dichloro-phenyl)-4-(4-methanesulfonylami-no-piperidin-1-yl)-butyric acid(intermediate 2)and (4-Chloro-benzyl)-methyl-amine(commerciallyavailable) | 546.2 | 0.0787 |
| 38 | 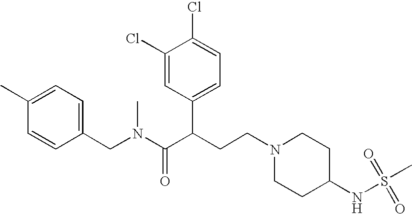 | 526.5 | 2-(3,4-Dichloro-phenyl)-4-(4-methannesulfonylamino-piperidin-1-yl)-N-methyl-N-(4-methyl-benzyl)-butyramide | 2-(3,4-Dichloro-phenyl)-4-(4-methanesulfonylami-no-piperidin-1-yl)-butyric acid(intermediate 2)and Methyl-(4-methyl-benzyl)-amine(commerciallyavailable) | 526.4 | 0.0929 |
| 39 | 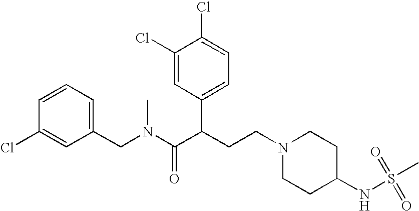 | 546.9 | N-(3-Chloro-benzyl)-2-(3,4-dichloro-phenyl)-4-(4-methanesulfonylamino-piperidin-1-yl)-N-methyl-butyramide | 2-(3,4-Dichloro-phenyl)-4-(4-methanesulfonylami-no-piperidin-1-yl)-butyric acid(intermediate 2)and (3-Chloro-benzyl)-methyl-amine(commerciallyavailable) | 546.2 | 0.2126 |
| 40 | 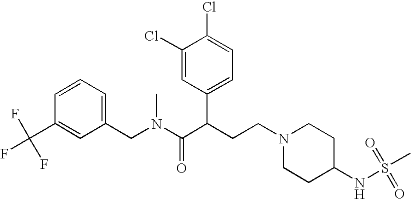 | 580.5 | 2-(3,4-Dichloro-phenyl)-4-(4-methanesulfonylamino-piperidin-1-yl)-N-methyl-N-(3-trifluoromethyl-benzyl)-butyramide | 2-(3,4-Dichloro-phenyl)-4-(4-methanesulfonylami-no-piperidin-1-yl)-butyric acid(intermediate 2)and Methyl-(3-trifluoromethyl-benzyl)-amine(commerciallyavailable) | 580.3 | 0.2801 |
| 41 | 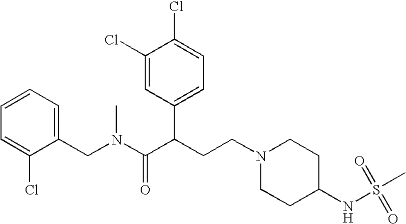 | 546.9 | N-(2-Chloro-benzyl)-2-(3,4-dichloro-phenyl)-4-(4-methanesulfonylamino-piperidin-1-yl)-N-methyl-butyramide | 2-(3,4-Dichloro-phenyl)-4-(4-methanesulfonylami-no-piperidin-1-yl)-butyric acid(intermediate 2)and (2-Chloro-benzyl)-methyl-amine(commerciallyavailable) | 546.2 | 0.2588 |
| 42 | 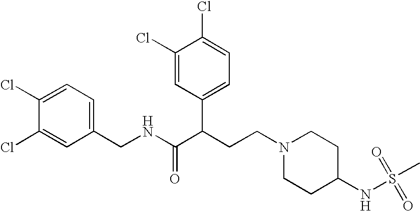 | 567.4 | N-(3,4-Dichloro-benzyl)-2-(3,4-dichloro-phenyl)-4-(4-methanesulfonylamino-piperidin-1-yl)-butyramide | 2-(3,4-Dichloro-phenyl)-4-(4-methanesulfonylami-no-piperidin-1-yl)-butyric acid(intermediate 2)and 3,4-Dichloro-benzylamine(commerciallyavailable) | 568.3 | 0.1919 |
| 43 | 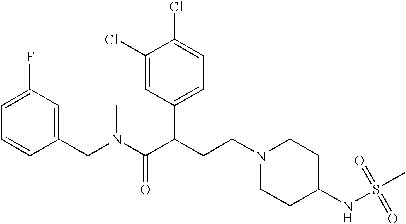 | 530.5 | 2-(3,4-Dichloro-phenyl)-N-(3-fluoro-benzyl)-4-(4-methanesulfonylamino-piperidin-1-yl)-N-methyl-butyramide | 2-(3,4-Dichloro-phenyl)-4-(4-methanesulfonylami-no-piperidin-1-yl)-butyric acid(intermediate 2)and (3-Fluloro-benzyl)-methyl-amine(commerciallyavailable) | 530.1 | 0.2544 |
| 44 | 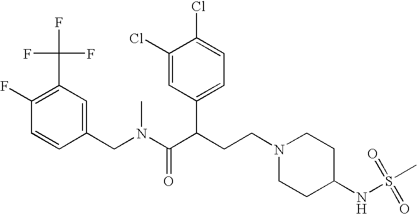 | 598.5 | 2-(3,4-Dichloro-phenyl)-N-(4-fluoro-3-trifluoromethyl-benzyl)-4-(4-methanesulfonylamino-piperidin-1-yl)-N-methyl-butyramide | 2-(3,4-Dichloro-phenyl)-4-(4-methanesulfonylami-no-piperidin-1-yl)-butyric acid(intermediate 2)and (4-Fluoro-3-trifluoromethyl-benzyl)-methyl-amine,hyrdochloride(intermediate 13) | 598.3 | 0.1323 |
| 45 | 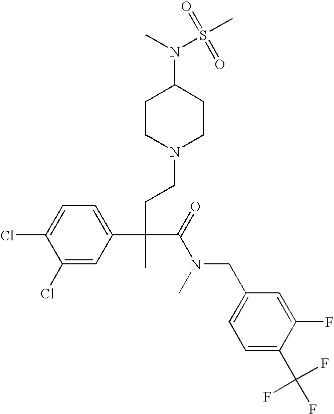 | 626.5 | 2-(3,4-Dichloro-phenyl)-N-(3-fluoro-4-trifluoromethyl-benzyl)-4-[4-(methanesulfonyl-methyl-amino)-piperidin-1-yl]-2,N-dimethyl-butyramide | 2-(3,4-Dichloro-phenyl)-4-[4-(methanesulfonyl-methyl-amino)-piperidin-1-yl]-2-methyl-butyric acid(intermediate 3)and (3-Fluoro-4-trifluoromethyl-benzyl)-methyl-amine,hydrochloride(intermediate 12) | 625.8 | 0.0085 |
| 46 | 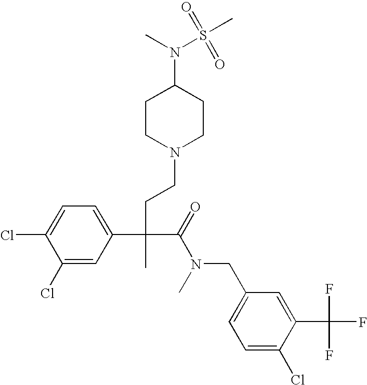 | 643.0 | N-(4-Chloro-3-trifluoromethyl-benzyl)-2-(3,4-dichloro-phenyl)-4-[4-(methanesulfonyl-methyl-amino)-piperidin-1-yl]-2,N-dimethyl-butyramide | 2-(3,4-Dichloro-phenyl)-4-[4-(methanesulfonyl-methyl-amino)-piperidin-1-yl]-2-methyl-butyric acid(intermediate 3)and (4-Chloro-3-trifluoromethyl-benzyl)-methyl-amine(commerciallyavailable) | 641.8 | 0.0024 |
| 47 | 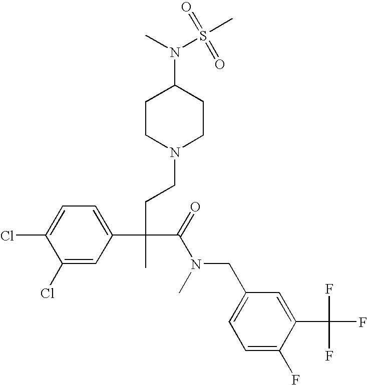 | 626.5 | 2-(3,4-Dichloro-phenyl)-N-(4-fluoro-3-trifluoromethyl-benzyl)-4-[4-(methanesulfonyl-methyl-amino)-piperidin-1-yl]-2,N-dimethyl-butyramide | 2-(3,4-Dichloro-phenyl)-4-[4-(methanesulfonyl-methyl-amino)-piperidin-1-yl]-2-methyl-butyric acid(intermediate 3)and (4-Fluoro-3-trifluoromethyl-benzyl)-methyl-amine,hydrochloride(intermediate 13) | 625.7 | 0.0021 |
| 48 | 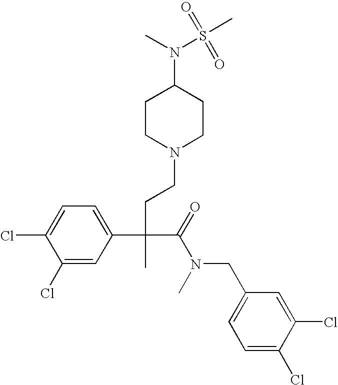 | 609.4 | N-(3,4-Dichloro-benzyl)-2-(3,4-dichloro-phenyl)-4-[4-(methanesulfonyl-methyl-amino)-piperidin-1-yl]-2,N-dimethyl-butyramide | 2-(3,4-Dichloro-phenyl)-4-[4-(methanesulfonyl-methyl-amino)-piperidin-1-yl]-2-methyl-butyric acid(intermediate 3)and (3,4-Dichloro-benzyl)-methyl-amine(commerciallyavailable) | 609.5 | 0.0036 |
| 49 | 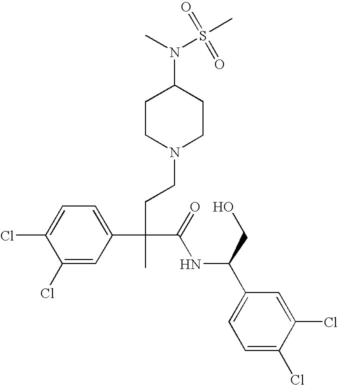 | 625.4 | 2-(3,4-Dichloro-phenyl)-N-[1-(3,4-dichloro-phenyl)-2-hydroxy-ethyl]-4-[4-(methanesulfonyl-methyl-amino)-piperidin-1-yl]-2-methyl-butyramide;diastereoisomer 1 | 2-(3,4-Dichloro-phenyl)-4-[4-(methanesulfonyl-methyl-amino)-piperidin-1-yl]-2-methyl-butyric acid(intermediate 3)and 2-Amino-2-(3,4-dichloro-phenyl)-ethanol(WO 2005058892) | 626.2 | 0.0055 |
| 50 | 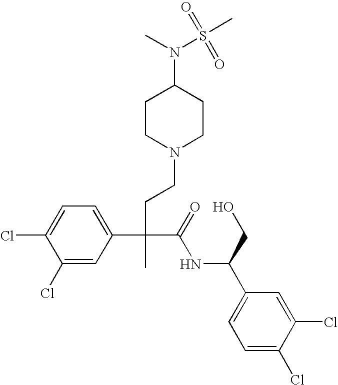 | 625.4 | 2-(3,4-Dichloro-phenyl)-N-[1-(3,4-dichloro-phenyl)-2-hydroxy-ethyl]-4-[4-(methanesulfonyl-methyl-amino)-piperidin-1-yl]-2-methyl-butyramide;diastereoisomer 2 | 2-(3,4-Dichloro-phenyl)-4-[4-(methanesulfonyl-methyl-amino)-piperidin-1-yl]-2-methyl-butyric acid(intermediate 3)and 2-Amino-2-(3,4-dichloro-phenyl)-ethanol(WO 2005058892) | 626.2 | 0.0521 |
| 51 | 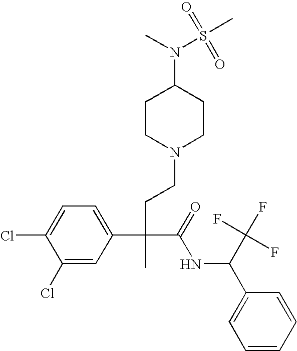 | 594.5 | 2-(3,4-Dichloro-phenyl)-4-[4-(methanesulfonyl-methyl-amino)-piperidin-1-yl]-2-methyl-N-(2,2,2-trifluoro-1-phenyl-ethyl)-butyramide | 2-(3,4-Dichloro-phenyl)-4-[4-(methanesulfonyl-methyl-amino)-piperidin-1-yl]-2-methyl-butyric acid(intermediate 3)and 2,2,2-Trifluoro-1-phenyl-ethylamine(commerciallyavailable) | 594.3 | 0.0187 |
| 52 | 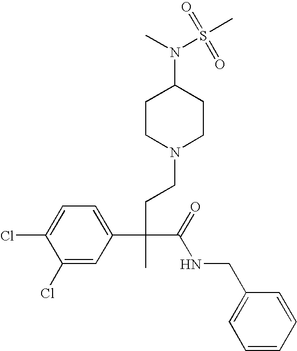 | 526.5 | N-Benzyl-2-(3,4-dichloro-phenyl)-4-[4-(methanesulfonyl-methyl-amino)-piperidin-1-yl]-2-methyl-butyramide | 2-(3,4-Dichloro-phenyl)-4-[4-(methanesulfonyl-methyl-amino)-piperidin-1-yl]-2-methyl-butyric acid(intermediate 3)and benzylamine(commerciallyavailable) | 525.8 | 0.0648 |
| 53 | 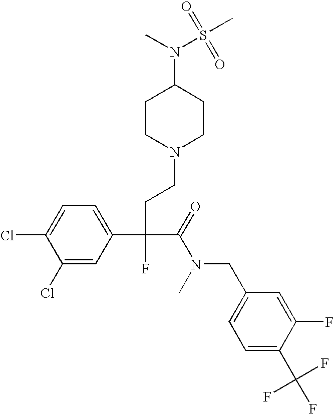 | 630.5 | 2-(3,4-Dichloro-phenyl)-2-fluoro-N-(3-fluoro-4-trifluoromethyl-benzyl)-4-[4-(methanesulfonyl-methyl-amino)-piperidin-1-yl]-N-methyl-butyramide | 2-(3,4-Dichloro-phenyl)-2-fluoro-4-[4-(methanesulfonyl-methyl-amino)-piperidin-1-yl]-butyric acid(intermediate 4)and (3-Fluoro-4-trifluoromethyl-benzyl)-methyl-amine,hydrochloride(intermediate 12) | 629.8 | 0.0183 |
| 54 | 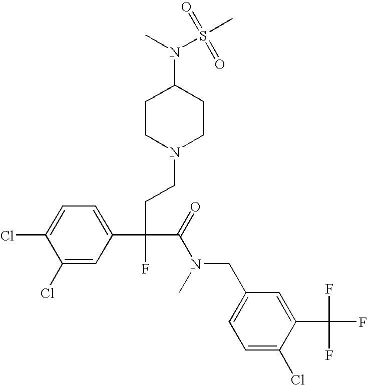 | 647.0 | N-(4-Chloro-3-trifluoromethyl-benzyl)-2-(3,4-dichloro-phenyl)-2-fluoro-4-[4-(methanesulfonyl-methyl-amino)-piperidin-1-yl]-N-methyl-butyramide | 2-(3,4-Dichloro-phenyl)-2-fluoro-4-[4-(methanesulfonyl-methyl-amino)-piperidin-1-yl]-butyric acid(intermediate 4)and (4-Chloro-3-trifluoromethyl-benzyl)-methyl-amine(commerciallyavailable) | 645.7 | 0.0245 |
| 55 | 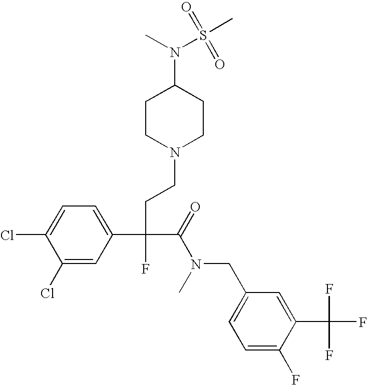 | 630.5 | 2-(3,4-Dichloro-phenyl)-2-fluoro-N-(4-fluoro-3-trifluoromethyl-benzyl)-4-[4-(methanesulfonyl-methyl-amino)-piperidin-1-yl]-N-methyl-butyramide | 2-(3,4-Dichloro-phenyl)-2-fluoro-4-[4-(methanesulfonyl-methyl-amino)-piperidin-1-yl]-butyric acid(intermediate 4)and (4-Fluoro-3-trifluoromethyl-benzyl)-methyl-amine,hyrdochloride(intermediate 13) | 629.8 | 0.0234 |
| 56 | 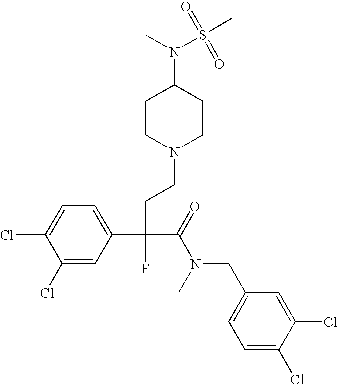 | 613.4 | N-(3,4-Dichloro-benzyl)-2-(3,4-dichloro-phenyl)-2-fluoro-4-[4-(methanesulfonyl-methyl-amino)-piperidin-1-yl]-N-methyl-butyramide | 2-(3,4-Dichloro-phenyl)-2-fluoro-4-[4-(methanesulfonyl-methyl-amino)-piperidin-1-yl]-butyric acid(intermediate 4)and (3,4-Dichloro-benzyl)-methyl-amine(commerciallyavailable) | 613.7 | 0.0228 |
| 57 | 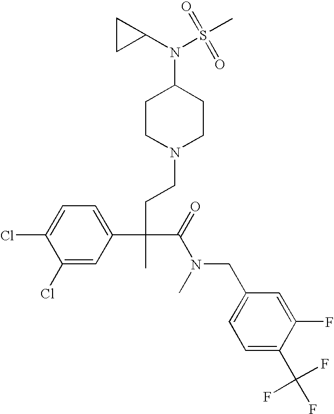 | 652.6 | 4-[4-(Cyclopropyl-methanesulfonyl-amino)-piperidin-1-yl]-2-(3,4-dichloro-phenyl)-N-(3-fluoro-4-trifluoromethyl-benzyl)-2,N-dimethyl-butyramide | 4-[4-(Cyclopropyl-methanesulfonyl-amino)-piperidin-1-yl]-2-(3,4-dichloro-phenyl)-2-methyl-butyric acid(intermediate 6)and (3-Fluoro-4-trifluoromethyl-benzyl)-methyl-amine,hydrochloride(intermediate 12) | 653.8 | 0.0059 |
| 58 | 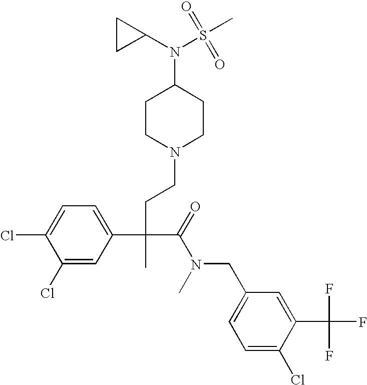 | 669.0 | N-(4-Chloro-3-trifluoromethyl-benzyl)-4-[4-(cyclopropyl-methanesulfonyl-amino)-piperidin-1-yl]-2-(3,4-dichloro-phenyl)-2,N-dimethyl-butyramide | 4-[4-(Cyclopropyl-methanesulfonyl-amino)-piperidin-1-yl]-2-(3,4-dichloro-phenyl)-2-methyl-butyric acid(intermediate 6)and (4-Chloro-3-trifluoromethyl-benzyl)-methyl-amine(commerciallyavailable) | 669.8 | 0.0036 |
| 59 | 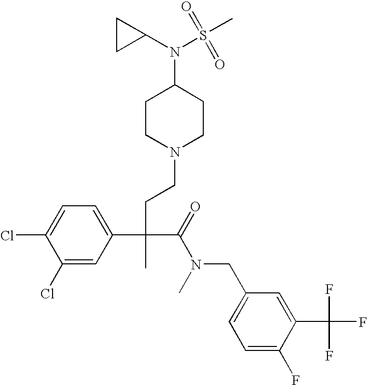 | 652.6 | 4-[4-(Cyclopropyl-methanesulfonyl-amino)-piperidin-1-yl]-2-(3,4-dichloro-phenyl)-N-(4-fluoro-3-trifluoromethyl-benzyl)-2,N-dimethyl-butyramide | 4-[4-(Cyclopropyl-methanesulfonyl-amino)-piperidin-1-yl]-2-(3,4-dichloro-phenyl)-2-methyl-butyric acid(intermediate 6)and (4-Fluoro-3-trifluoromethyl-benzyl)-methyl-amine,hyrdochloride(intermediate 13) | 653.7 | 0.0043 |
| 60 | 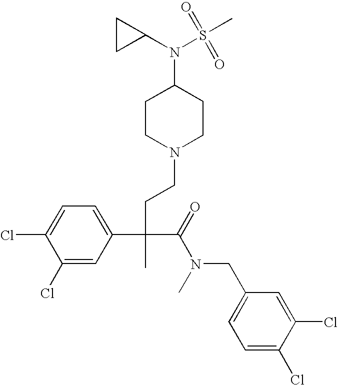 | 635.5 | 4-[4-(Cyclopropyl-methanesulfonyl-amino)-piperidin-1-yl]-N-(3,4-dichloro-benzyl)-2-(3,4-dichloro-phenyl)-2,N-dimethyl-butyramide | 4-[4-(Cyclopropyl-methanesulfonyl-amino)-piperidin-1-yl]-2-(3,4-dichloro-phenyl)-2-methyl-butyric acid(intermediate 6)and (3,4-Dichloro-benzyl)-methyl-amine(commerciallyavailable) | 635.5 | 0.0026 |
| 61 | 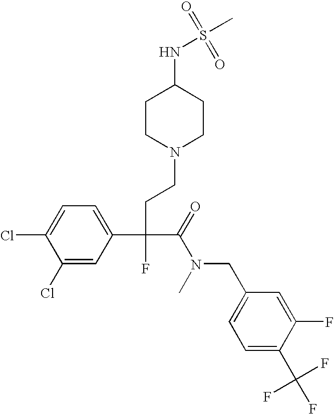 | 616.5 | 2-(3,4-Dichloro-phenyl)-2-fluoro-N-(3-fluoro-4-trifluoromethyl-benzyl)-4-(4-methanesulfonylamino-piperidin-1-yl)-N-methyl-butyramide | 2-(3,4-Dichloro-phenyl)-2-fluoro-4-(4-methanesulfonylami-no-piperidin-1-yl)-butyric acid(intermediate 7)and (3-Fluoro-4-trifluoromethyl-benzyl)-methyl-amine,hydrochloride(intermediate 12) | 616.1 | 0.08 |
| 62 | 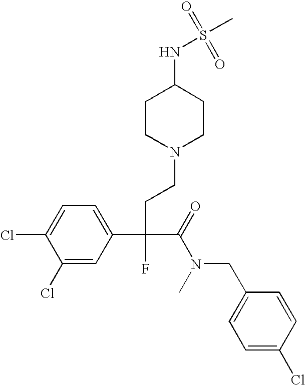 | 564.9 | N-(4-Chloro-benzyl)-2-(3,4-dichloro-phenyl)-2-fluoro-4-(4-methanesulfonylamino-piperidin-1-yl)-N-methyl-butyramide | 2-(3,4-Dichloro-phenyl)-2-fluoro-4-(4-methanesulfonylami-no-piperidin-1-yl)-butyric acid(intermediate 7)and (4-Chloro-benzyl)-methyl-amine(commerciallyavailable) | 564.2 | 0.1052 |
| 63 | 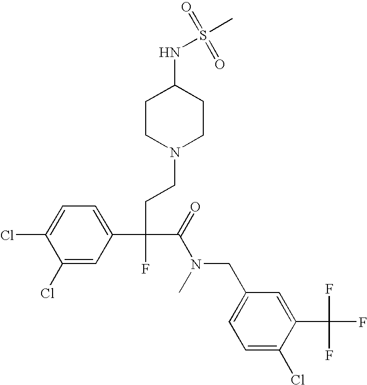 | 632.9 | N-(4-Chloro-3-trifluoromethyl-benzyl)-2-(3,4-dichloro-phenyl)-2-fluoro-4-(4-methanesulfonylamino-piperidin-1-yl)-N-methyl-butyramide | 2-(3,4-Dichloro-phenyl)-2-fluoro-4-(4-methanesulfonylami-no-piperidin-1-yl)-butyric acid(intermediate 7)and (4-Chloro-3-trifluoromethyl-benzyl)-methyl-amine(commerciallyavailable) | 632.2 | 0.1169 |
| 64 | 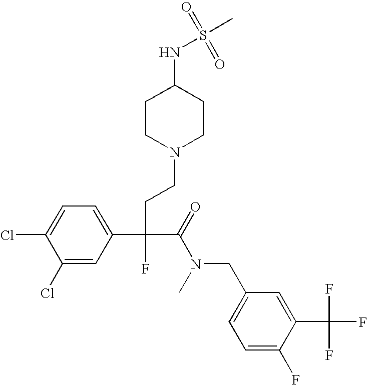 | 616.5 | 2-(3,4-Dichloro-phenyl)-2-fluoro-N-(4-fluoro-3-trifluoromethyl-benzyl)-4-(4-methanesulfonylamino-piperidin-1-yl)-N-methyl-butyramide | 2-(3,4-Dichloro-phenyl)-2-fluoro-4-(4-methanesulfonylami-no-piperidin-1-yl)-butyric acid(intermediate 7)and (4-Fluoro-3-trifluoromethyl-benzyl)-methyl-amine,hyrdochloride(intermediate 13) | 616.1 | 0.1219 |
| 65 | 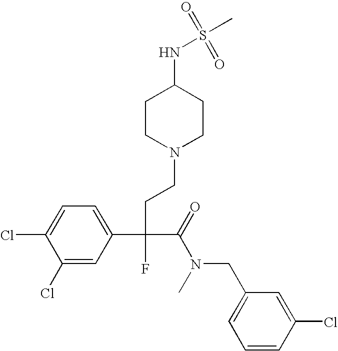 | 564.9 | N-(3-Chloro-benzyl)-2-(3,4-dichloro-phenyl)-2-fluoro-4-(4-methanesulfonylamino-piperidin-1-yl)-N-methyl-butyramide | 2-(3,4-Dichloro-phenyl)-2-fluoro-4-(4-methanesulfonylami-no-piperidin-1-yl)-butyric acid(intermediate 7)and (3-Chloro-benzyl)-methyl-amine(commerciallyavailable) | 564.2 | 0.3067 |
| 66 | 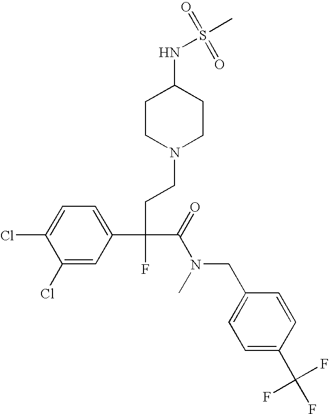 | 598.5 | 2-(3,4-Dichloro-phenyl)-2-fluoro-4-(4-methanesulfonylamino-piperidin-1-yl)-N-methyl-N-(4-trifluoromethyl-benzyl)-butyramide | 2-(3,4-Dichloro-phenyl)-2-fluoro-4-(4-methanesulfonylami-no-piperidin-1-yl)-butyric acid(intermediate 7)and Methyl-(4-trifluoromethyl-benzyl)-amine(commerciallyavailable) | 598.3 | 0.0843 |
| 67 | 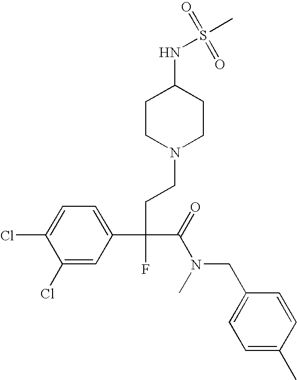 | 544.5 | 2-(3,4-Dichloro-phenyl)-2-fluoro-4-(4-methanesulfonylamino-piperidin-1-yl)-N-methyl-N-(4-methyl-benzyl)-butyramide | 2-(3,4-Dichloro-phenyl)-2-fluoro-4-(4-methanesulfonylami-no-piperidin-1-yl)-butyric acid(intermediate 7)and Methyl-(4-methyl-benzyl)-amine(commerciallyavailable) | 546.2 | 0.3227 |
| 68 | 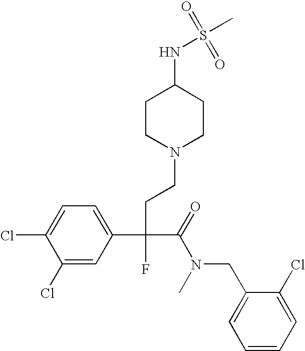 | 564.9 | N-(2-Chloro-benzyl)-2-(3,4-dihcloro-phenyl)-2-fluoro-4-(4-methanesulfonylamino-piperidin-1-yl)-N-methyl-butyramide | 2-(3,4-Dichloro-phenyl)-2-fluoro-4-(4-methanesulfonylami-no-piperidin-1-yl)-butyric acid(intermediate 7)and (2-Chloro-benzyl)-methyl-amine(commerciallyavailable) | 564.2 | 0.1585 |
| 69 | 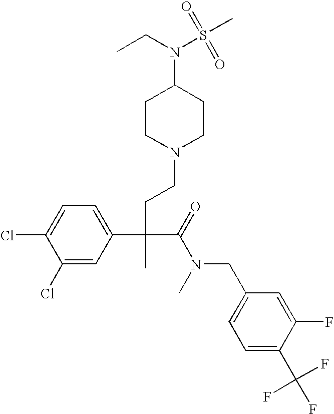 | 640.6 | 2-(3,4-Dichloro-phenyl)-4-[4-(ethyl-methanesulfonyl-amino)-piperidin-1-yl]-N-(3-fluoro-4-trifluoromethyl-benzyl)-2,N-dimethyl-butyramide | 2-(3,4-Dichloro-phenyl)-4-[4-(ethyl-methanesulfonyl-amino)-piperidin-1-yl]-2-methyl-butyric acid(intermediate 9)and (3-Fluoro-4-trifluoromethyl-benzyl)-methyl-amine,hydrochloride(intermediate 12) | 640.3 | 0.0024 |
| 70 | 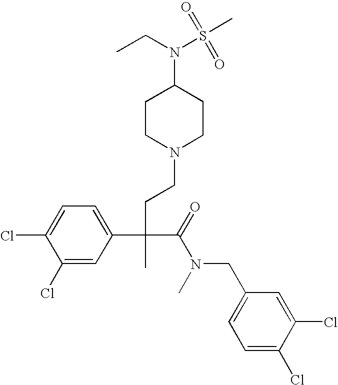 | 623.5 | N-(3,4-Dichloro-benzyl)-2-(3,4-dichloro-phenyl)-4-[4-(ethyl-methanesulfonyl-amino)-piperidin-1-yl]-2,N-dimethyl-butyramide | 2-(3,4-Dichloro-phenyl)-4-[4-(ethyl-methanesulfonyl-amino)-piperidin-1-yl]-2-methyl-butyric acid(intermediate 9)and (3,4-Dichloro-benzyl)-methyl-amine(commerciallyavailable) | 624.3 | 0.0012 |
| 71 | 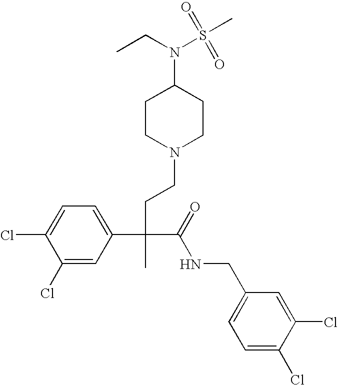 | 609.4 | N-(3,4-Dichloro-benzyl)-2-(3,4-dichloro-phenyl)-4-[4-(ethyl-methanesulfonyl-amino)-piperidin-1-yl]-2-methyl-butyramide | 2-(3,4-Dichloro-phenyl)-4-[4-(ethyl-methanesulfonyl-amino)-piperidin-1-yl]-2-methyl-butyric acid(intermediate 9)and 3,4-Dichloro-benzylamine(commerciallyavailable) | 610.1 | 0.0028 |
| 72 | 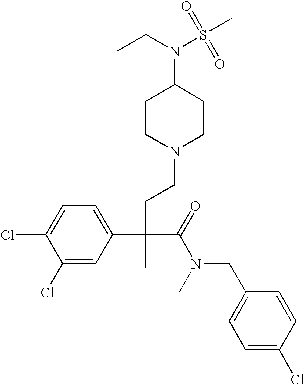 | 589.0 | N-(4-Chloro-benzyl)-2-(3,4-dichloro-phenyl)-4-[4-(ethyl-methanesulfonyl-amino)-piperidin-1-yl]-2,N-dimethyl-butyramide | 2-(3,4-Dichloro-phenyl)-4-[4-(ethyl-methanesulfonyl-amino)-piperidin-1-yl]-2-methyl-butyric acid(intermediate 9)and (4-Chloro-benzyl)-methyl-amine(commerciallyavailable) | 590.2 | 0.0022 |
| 73 | 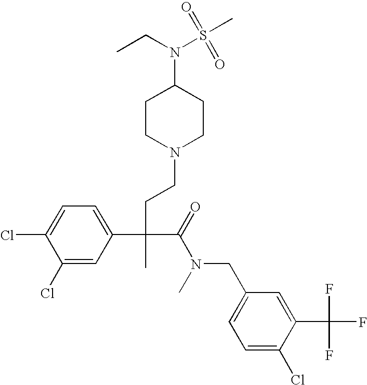 | 657.0 | N-(4-Chloro-3-trifluoromethyl-benzyl)-2-(3,4-dichloro-phenyl)-4-[4-(ethyl-methanesulfonyl-amino)-piperidin-1-yl]-2,N-dimethyl-butyramide | 2-(3,4-Dichloro-phenyl)-4-[4-(ethyl-methanesulfonyl-amino)-piperidin-1-yl]-2-methyl-butyric acid(intermediate 9)and 4-Chloro-3-trifluoromethyl-benzylamine(commerciallyavailable) | 658.3 | 0.0015 |
| 74 | 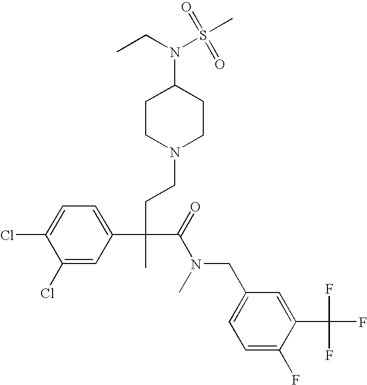 | 640.6 | 2-(3,4-Dichloro-phenyl)-4-[4-(ethyl-methanesulfonyl-amino)-piperidin-1-yl]-N-(4-fluoro-3-trifluoromethyl-benzyl)-2,N-dimethyl-butyramide | 2-(3,4-Dichloro-phenyl)-4-[4-(ethyl-methanesulfonyl-amino)-piperidin-1-yl]-2-methyl-butyric acid(intermediate 9)and (4-Fluoro-3-trifluoromethyl-benzyl)-methyl-amine,hyrdochloride(intermediate 13) | 640.2 | 0.0013 |
| 75 | 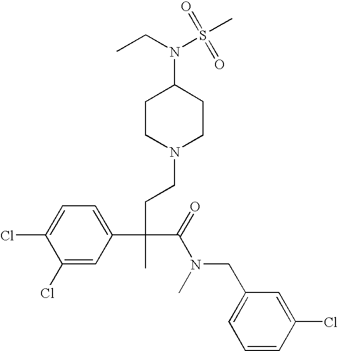 | 589.0 | N-(3-Chloro-benzyl)-2-(3,4-dichloro-phenyl)-4-[4-(ethyl-methanesulfonyl-amino)-piperidin-1-yl]-2,N-dimethyl-butyramide | 2-(3,4-Dichloro-phenyl)-4-[4-(ethyl-methanesulfonyl-amino)-piperidin-1-yl]-2-methyl-butyric acid(intermediate 9)and (3-Chloro-benzyl)-methyl-amine(commerciallyavailable) | 590.2 | 0.0039 |
| 76 | 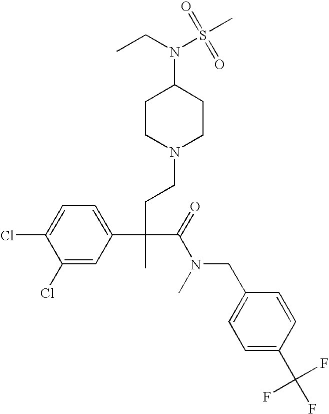 | 622.6 | 2-(3,4-Dichloro-phenyl)-4-[4-(ethyl-methanesulfonyl-amino)-piperidin-1-yl]-2,N-dimethyl-N-(4-trifluoromethyl-benzyl)-butyramide | 2-(3,4-Dichloro-phenyl)-4-[4-(ethyl-methanesulfonyl-amino)-piperidin-1-yl]-2-methyl-butyric acid(intermediate 9)and Methyl-(4-trifluoromethyl-benzyl)-amine(commerciallyavailable) | 622.4 | 0.0017 |
| 77 | 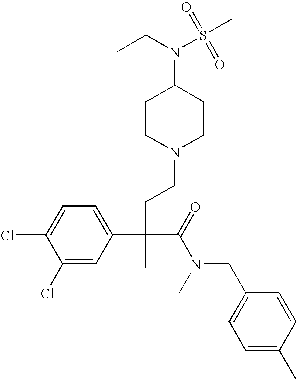 | 568.6 | 2-(3,4-Dichloro-phenyl)-4-[4-(ethyl-methanesulfonyl-amino)-piperidin-1-yl]-2,N-dimethyl-N-(4-methyl-benzyl)-butyramide | 2-(3,4-Dichloro-phenyl)-4-[4-(ethyl-methanesulfonyl-amino)-piperidin-1-yl]-2-methyl-butyric acid(intermediate 9)and Methyl-(4-methyl-benzyl)-amine(commerciallyavailable) | 568.3 | 0.0031 |
| 78 | 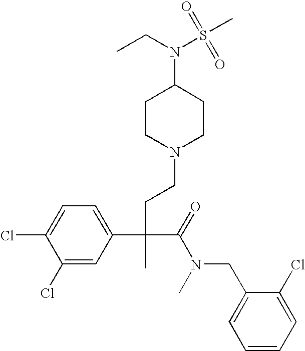 | 589.0 | N-(2-Chloro-benzyl)-2-(3,4-dichloro-phenyl)-4-[4-(ethyl-methanesulfonyl-amino)-piperidin-1-yl]-2,N-dimethyl-butyramide | 2-(3,4-Dichloro-phenyl)-4-[4-(ethyl-methanesulfonyl-amino)-piperidin-1-yl]-2-methyl-butyric acid(intermediate 9)and (2-Chloro-benzyl)-methyl-amine(commerciallyavailable) | 590.2 | 0.0036 |
| 79 | 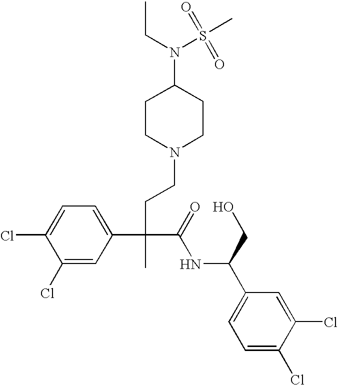 | 639.5 | 2-(3,4-Dichloro-phenyl)-N-[1-(3,4-dichloro-phenyl)-2-hydroxy-ethyl]-4-[4-(ethyl-methanesulfonyl-amino)-piperidin-1-yl]-2-methyl-butyramide;diastereoisomer 1 | 2-(3,4-Dichloro-phenyl)-4-[4-(ethyl-methanesulfonyl-amino)-piperidin-1-yl]-2-methyl-butyric acid(intermediate 9)and 2-Amino-2-(3,4-dichloro-phenyl)-ethanol(WO 2005058892) | 640.3 | 0.0015 |
| 80 | 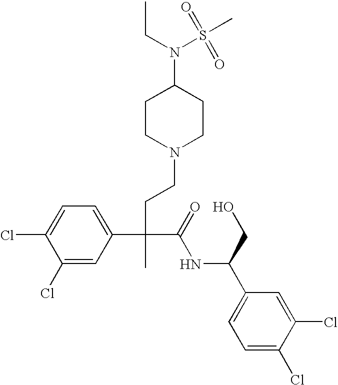 | 639.5 | 2-(3,4-Dichloro-phenyl)-N-[1-(3,4-dichloro-phenyl)-2-hydroxy-ethyl]-4-[4-(ethyl-methanesulfonyl-amino)-piperidin-1-yl]-2-methyl-butyramide;diastereoisomer 2 | 2-(3,4-Dichloro-phenyl)-4-[4-(ethyl-methanesulfonyl-amino)-piperidin-1-yl]-2-methyl-butyric acid(intermediate 9)and 2-Amino-2-(3,4-dichloro-phenyl)-ethanol(WO 2005058892) | 640.2 | 0.0133 |
| 81 | 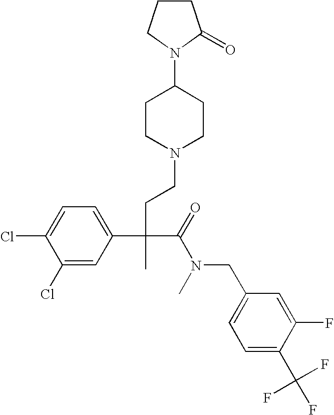 | 602.5 | 2-(3,4-Dichloro-phenyl)-N-(3-fluoro-4-trifluoromethyl-benzyl)-2,N-dimethyl-4-[4-(2-oxo-pyrrolidin-1-yl)-piperidin-1-yl]-butyramide | 2-(3,4-Dichloro-phenyl)-2-methyl-4-[4-(2-oxo-pyrrolidin-1-yl)-piperidin-1-yl]-butyric acid(intermediate 10)and (3-Fluoro-4-trifluoromethyl-benzyl)-methyl-amine,hydrochloride(intermediate 12) | 601.9 | 0.003 |
| 82 | 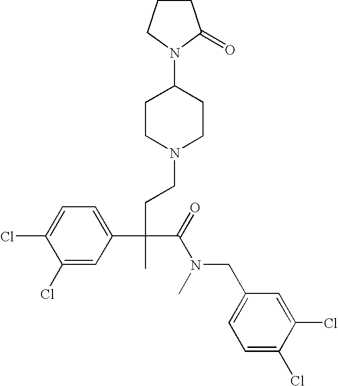 | 585.4 | N-(3,4-Dichloro-benzyl)-2-(3,4-dichloro-phenyl)-2,N-dimethyl-4-[4-(2-oxo-pyrrolidin-1-yl)-piperidin-1-yl]-butyramide | 2-(3,4-Dichloro-phenyl)-2-methyl-4-[4-(2-oxo-pyrrolidin-1-yl)-piperidin-1-yl]-butyric acid(intermediate 10)and (3,4-Dichloro-benzyl)-methyl-amine(commerciallyavailable) | 585.7 | 0.0012 |
| 83 | 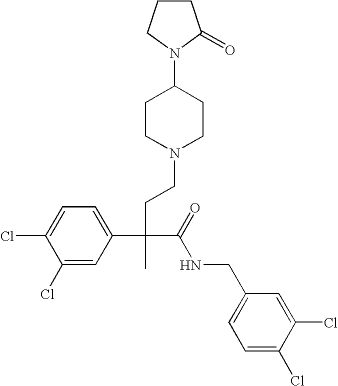 | 571.4 | N-(3,4-Dichloro-benzyl)-2-(3,4-dichloro-phenyl)-2-methyl-4-[4-(2-oxo-pyrrolidin-1-yl)-piperidin-1-yl]-butyramide | 2-(3,4-Dichloro-phenyl)-2-methyl-4-[4-(2-oxo-pyrrolidin-1-yl)-piperidin-1-yl]-butyric acid(intermediate 10)and 3,4-Dichloro-benzylamine(commerciallyavailable) | 569.7 | 0.0023 |
| 84 | 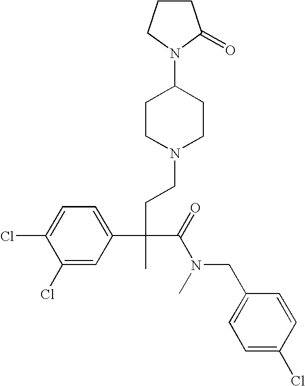 | 551.0 | N-(4-Chloro-benzyl)-2-(3,4-dichloro-phenyl)-2,N-dimethyl-4-[4-(2-oxo-pyrrolidin-1-yl)-piperidin-1-yl]-butyramide | 2-(3,4-Dichloro-phenyl)-2-methyl-4-[4-(2-oxo-pyrrolidin-1-yl)-piperidin-1-yl]-butyric acid(intermediate 10)and (4-Chloro-benzyl)-methyl-amine(commerciallyavailable) | 549.9 | 0.0021 |
| 85 | 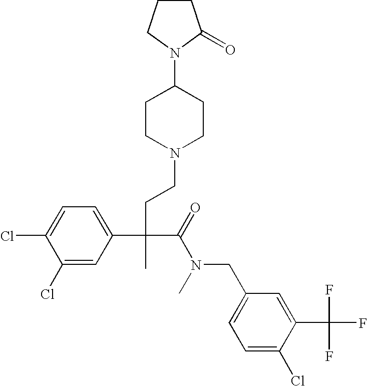 | 619.0 | N-(4-Chloro-3-trifluoromethyl-benzyl)-2-(3,4-dichloro-phenyl)-2,N-dimethyl-4-[4-(2-oxo-pyrrolidin-1-yl)-piperidin-1-yl]-butyramide | 2-(3,4-Dichloro-phenyl)-2-methyl-4-[4-(2-oxo-pyrrolidin-1-yl)-piperidin-1-yl]-butyric acid(intermediate 10)and 4-Chloro-3-trifluoromethyl-benzylamine(commerciallyavailable) | 619.7 | 0.0014 |
| 86 | 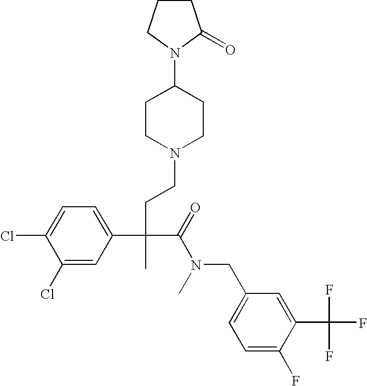 | 602.5 | 2-(3,4-Dichloro-phenyl)-N-(4-fluoro-3-trifluoromethyl-benzyl)-2,N-dimethyl-4-[4-(2-oxo-pyrrolidin-1-yl)-piperidin-1-yl]-butyramide | 2-(3,4-Dichloro-phenyl)-2-methyl-4-[4-(2-oxo-pyrrolidin-1-yl)-piperidin-1-yl]-butyric acid(intermediate 10)and (4-Fluoro-3-trifluoromethyl-benzyl)-methyl-amine,hyrdochloride(intermediate 13) | 601.9 | 0.0014 |
| 87 | 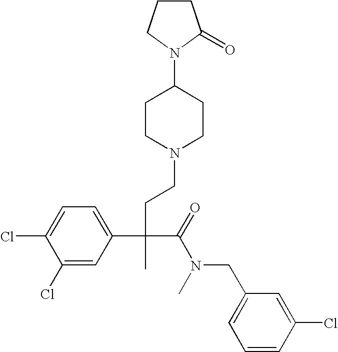 | 551.0 | N-(3-Chloro-benzyl)-2-(3,4-dichloro-phenyl)-2,N-dimethyl-4-[4-(2-oxo-pyrrolidin-1-yl)-piperidin-1-yl]-butyramide | 2-(3,4-Dichloro-phenyl)-2-methyl-4-[4-(2-oxo-pyrrolidin-1-yl)-piperidin-1-yl]-butyric acid(intermediate 10)and (3-Chloro-benzyl)-methyl-amine(commerciallyavailable) | 549.9 | 0.0046 |
| 88 | 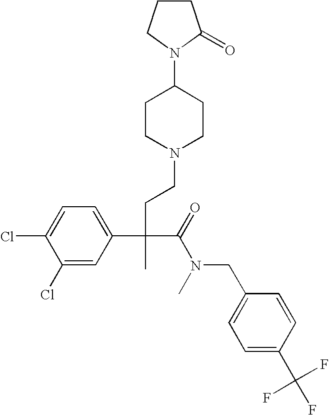 | 584.5 | 2-(3,4-Dichloro-phenyl)-2,N-dimethyl-4-[4-(2-oxo-pyrrolidin-1-yl)-piperidin-1-yl]-N-(4-trifluoromethyl-benzyl)-butyramide | 2-(3,4-Dichloro-phenyl)-2-methyl-4-[4-(2-oxo-pyrrolidin-1-yl)-piperidin-1-yl]-butyric acid(intermediate 10)and Methyl-(4-trifluoromethyl-benzyl)-amine(commerciallyavailable) | 583.7 | 0.0018 |
| 89 | 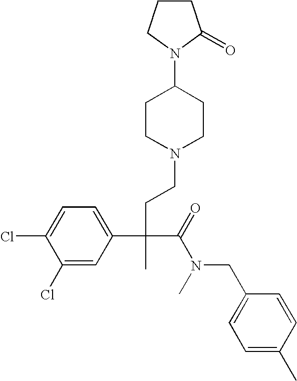 | 530.5 | 2-(3,4-Dichloro-phenyl)-2,N-dimethyl-N-(4-methyl-benzyl)-4-[4-(2-oxo-pyrrolidin-1-yl)-piperidin-1-yl]-butyramide | 2-(3,4-Dichloro-phenyl)-2-methyl-4-[4-(2-oxo-pyrrolidin-1-yl)-piperidin-1-yl]-butyric acid(intermediate 10)and Methyl-(4-methyl-benzyl)-amine(commerciallyavailable) | 529.9 | 0.0053 |
| 90 | 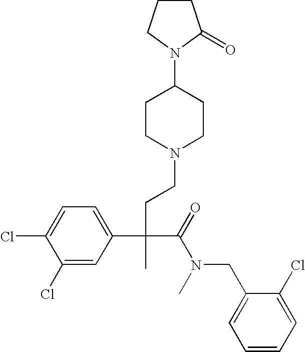 | 551.0 | N-(2-Chloro-benzyl)-2-(3,4-dichloro-phenyl)-2,N-dimethyl-4-[4-(2-oxo-pyrrolidin-1-yl)-piperidin-1-yl]-butyramide | 2-(3,4-Dichloro-phenyl)-2-methyl-4-[4-(2-oxo-pyrrolidin-1-yl)-piperidin-1-yl]-butyric acid(intermediate 10)and (3-Chloro-benzyl)-methyl-amine(commerciallyavailable) | 551.8 | 0.0025 |
| 91 | 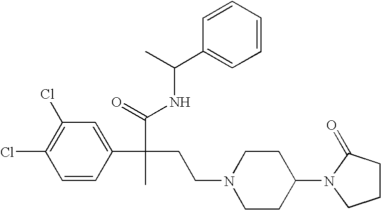 | 516.5 | 2-(3,4-Dichloro-phenyl)-2-methyl-4-[4-(2-oxo-pyrrolidin-1-yl)-piperidin-1-yl]-N-(1-phenyl-ethyl)-butyramide | 2-(3,4-Dichloro-phenyl)-2-methyl-4-[4-(2-oxo-pyrrolidin-1-yl)-piperidin-1-yl]-butyric acid(intermediate 10)and 1-Phenyl-ethylamine(commerciallyavailable) | 516.2 | 0.0512 |
| 92 | 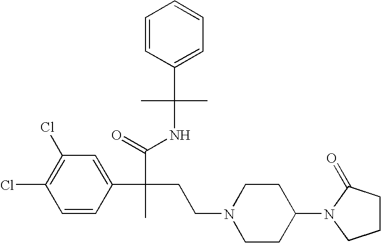 | 530.5 | 2-(3,4-Dichloro-phenyl)-2-methyl-N-(1-methyl-1-phenyl-ethyl)-4-[4-(2-oxo-pyrrolidin-1-yl)-piperidin-1-yl]-butyramide | 2-(3,4-Dichloro-phenyl)-2-methyl-4-[4-(2-oxo-pyrrolidin-1-yl)-piperidin-1-yl]-butyric acid(intermediate 10)and 1-Methyl-1-phenyl-ethylamine(commerciallyavailable) | 530.2 | 0.9912 |
| 93 | 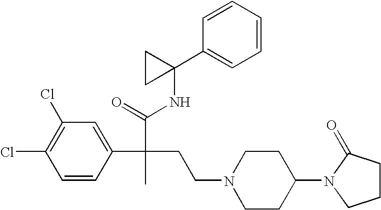 | 528.5 | 2-(3,4-Dichloro-phenyl)-2-methyl-4-[4-(2-oxo-pyrrolidin-1-yl)-piperidin-1-yl]-N-(1-phenyl-cyclopropyl)-butyramide | 2-(3,4-Dichloro-phenyl)-2-methyl-4-[4-(2-oxo-pyrrolidin-1-yl)-piperidin-1-yl]-butyric acid(intermediate 10)and 1-Phenyl-cyclopropylamine(commerciallyavailable) | 528.1 | 0.1842 |
| 94 | 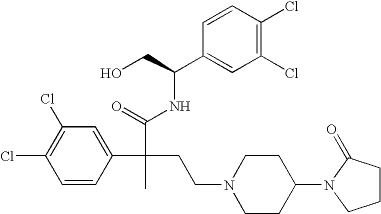 | 601.4 | 2-(3,4-Dichloro-phenyl)-N-[-1-(3,4-dichloro-phenyl)-2-hydroxy-ethyl]-2-methyl-4-[4-(2-oxo-pyrrolidin-1-yl)-piperidin-1-yl]-butyramide;diastereoisomer 1 | 2-(3,4-Dichloro-phenyl)-2-methyl-4-[4-(2-oxo-pyrrolidin-1-yl)-piperidin-1-yl]-butyric acid(intermediate 10)and 2-Amino-2-(3,4-dichloro-phenyl)-ethanol(WO 2005058892) | 602.1 | 0.003 |
| 95 | 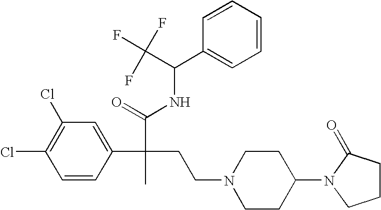 | 570.5 | 2-(3,4-Dichloro-phenyl)-2-methyl-4-[4-(2-oxo-pyrrolidin-1-yl)-piperidin-1-yl]-N-(2,2,2-trifluoro-1-phenyl-ethyl)-butyramide | 2-(3,4-Dichloro-phenyl)-2-methyl-4-[4-(2-oxo-pyrrolidin-1-yl)-piperidin-1-yl]-butyric acid(intermediate 10)and 2,2,2-Trifluoro-1-phenyl-ethylamine(commerciallyavailable) | 572.2 | 0.0047 |
| 96 | 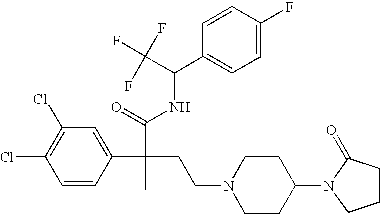 | 588.5 | 2-(3,4-Dichloro-phenyl)-2-methyl-4-[4-(2-oxo-pyrrolidin-1-yl)-piperidin-1-yl]-N-[2,2,2-trifluoro-1-(4-fluoro-phenyl)-ethyl]-butyramide | 2-(3,4-Dichloro-phenyl)-2-methyl-4-[4-(2-oxo-pyrrolidin-1-yl)-piperidin-1-yl]-butyric acid(intermediate 10)and 2,2,2-Trifluoro-1-(4-fluoro-phenyl)-ethylamine(commerciallyavailable) | 588.3 | 0.0072 |
| 97 | 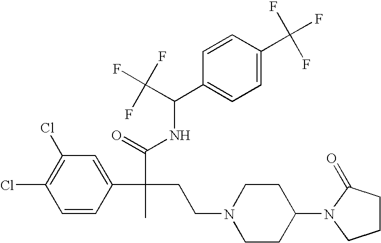 | 638.5 | 2-(3,4-Dichloro-phenyl)-2-methyl-4-[4-(2-oxo-pyrrolidin-1-yl)-piperidin-1-yl]-N-[2,2,2-trifluoro-1-(4-trifluoromethyl-phenyl)-ethyl]-butyramide | 2-(3,4-Dichloro-phenyl)-2-methyl-4-[4-(2-oxo-pyrrolidin-1-yl)-piperidin-1-yl]-butyric acid(intermediate 10)and 2,2,2-Trifluoro-1-(4-trifluromethyl-phenyl)-ethylamine(commerciallyavailable) | 638.2 | 0.0081 |
| 98 | 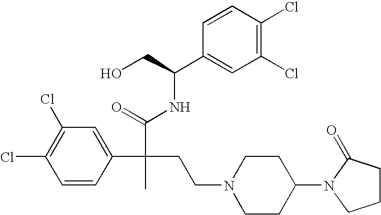 | 601.4 | 2-(3,4-Dichloro-phenyl)-N-[1-(3,4-dichloro-phenyl)-2-hydroxy-ethyl]-2-methyl-4-[4-(2-oxo-pyrrolidin-1-yl)-piperidin-1-yl]-butyramide;diastereoisomer 2 | 2-(3,4-Dichloro-phenyl)-2-methyl-4-[4-(2-oxo-pyrrolidin-1-yl)-piperidin-1-yl]-butyric acid(intermediate 10)and 2-Amino-2-(3,4-dichloro-phenyl)-ethanol(WO 2005058892) | 602.1 | 0.0499 |
| 99 | 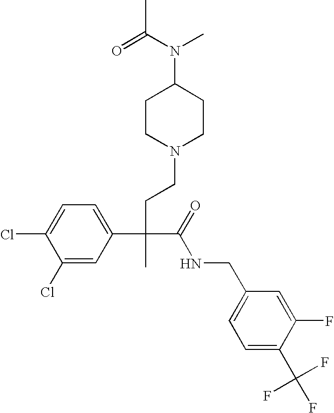 | 590.5 | 4-[4-(Acetyl-methyl-amino)-piperidin-1-yl]-2-(3,4-dichloro-phenyl)-N-(3-fluoro-4-trifluoromethyl-benzyl)-2-methyl-butyramide | 4-[4-(Acetyl-methyl-amino)-piperidin-1-yl]-2-(3,4-dichloro-phenyl)-2-methyl-butyric acid(intermediate 11)and 3-Fluoro-4-trifluoromethyl-benzylamine(commerciallyavailable) | 590.3 | 0.0058 |
| 100 | 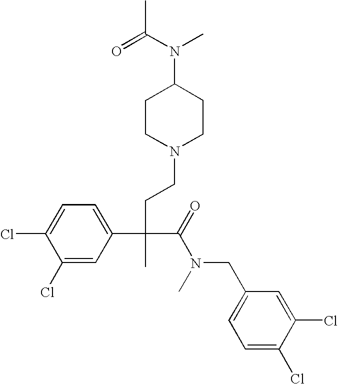 | 573.4 | 4-[4-(Acetyl-methyl-amino)-piperidin-1-yl]-N-(3,4-dichloro-benzyl)-2-(3,4-dichloro-phenyl)-2,N-dimethyl-butyramide | 4-[4-(Acetyl-methyl-amino)-piperidin-1-yl]-2-(3,4-dichloro-phenyl)-2-methyl-butyric acid(intermediate 11)and (3,4-Dichloro-benzyl)-methyl-amine(commerciallyavailable) | 574.2 | 0.003 |
| 101 | 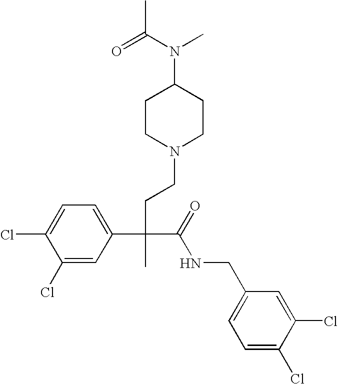 | 559.4 | 4-[4-(Acetyl-methyl-amino)-piperidin-1-yl]-N-(3,4-dichloro-benzyl)-2-(3,4-dichloro-phenyl)-2-methyl-butyramide | 4-[4-(Acetyl-methyl-amino)-piperidin-1-yl]-2-(3,4-dichloro-phenyl)-2-methyl-butyric acid(intermediate 11)and 3,4-Dichloro-benzylamine(commerciallyavailable) | 560.2 | 0.0068 |
| 102 | 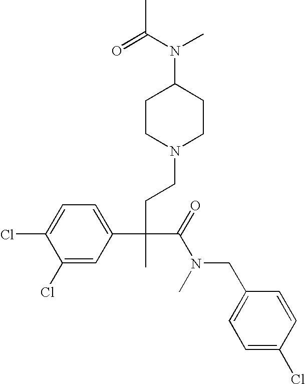 | 539.0 | 4-[4-(Acetyl-methyl-amino)-piperidin-1-yl]-N-(4-chloro-benzyl)-2-(3,4-dichloro-phenyl)-2,N-dimethyl-butyramide | 4-[4-(Acetyl-methyl-amino)-piperidin-1-yl]-2-(3,4-dichloro-phenyl)-2-methyl-butyric acid(intermediate 11)and (4-Chloro-benzyl)-methyl-amine(commerciallyavailable) | 538.3 | 0.0051 |
| 103 | 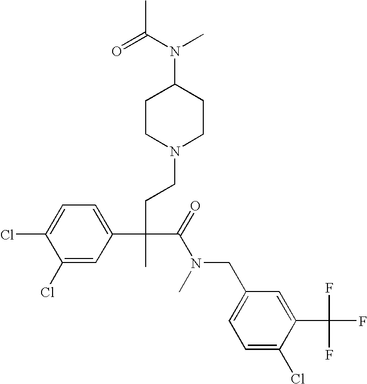 | 606.9 | 4-[4-(Acetyl-methyl-amino)-piperidin-1-yl]-N-(4-chloro-3-trifluoromethyl-benzyl)-2-(3,4-dichloro-phenyl)-2,N-dimethyl-butyramide | 4-[4-(Acetyl-methyl-amino)-piperidin-1-yl]-2-(3,4-dichloro-phenyl)-2-methyl-butyric acid(intermediate 11)and (4-Chloro-3-trifluoromethyl-benzyl)-methyl-amine(commerciallyavailable) | 608.2 | 0.0022 |
| 104 | 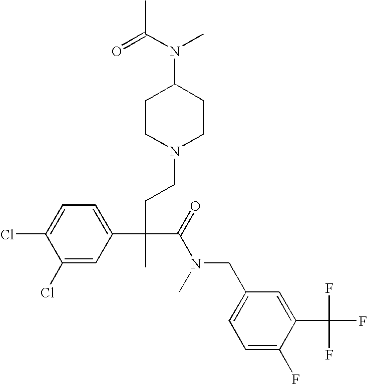 | 590.5 | 4-[4-(Acetyl-methyl-amino)-piperidin-1-yl]-2-(3,4-dichloro-phenyl)-N-(4-fluoro-3-trifluoromethyl-benzyl)-2,N-dimethyl-butyramide | 4-[4-(Acetyl-methyl-amino)-piperidin-1-yl]-2-(3,4-dichloro-phenyl)-2-methyl-butyric acid(intermediate 11)and (4-Fluoro-3-trifluoromethyl-benzyl)-methyl-amine,hyrdochloride(intermediate 13) | 590.3 | 0.0023 |
| 105 | 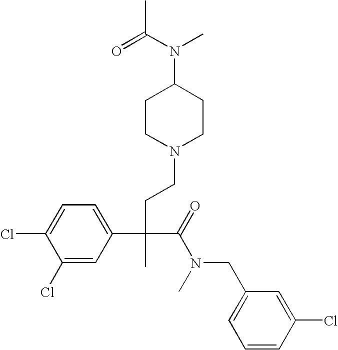 | 539.0 | 4-[4-(Acetyl-methyl-amino)-piperidin-1-yl]-N-(3-chloro-benzyl)-2-(3,4-dichloro-phenyl)-2,N-dimethyl-butyramide | 4-[4-(Acetyl-methyl-amino)-piperidin-1-yl]-2-(3,4-dichloro-phenyl)-2-methyl-butyric acid(intermediate 11)and (3-Chloro-benzyl)-methyl-amine(commerciallyavailable) | 538.3 | 0.0087 |
| 106 | 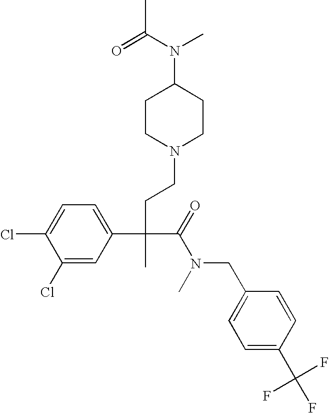 | 572.5 | 4-[4-(Acetyl-methyl-amino)-piperidin-1-yl]-2-(3,4-dichloro-phenyl)-2,N-dimethyl-N-(4-trifluoromethyl-benzyl)-butyramide | 4-[4-(Acetyl-methyl-amino)-piperidin-1-yl]-2-(3,4-dichloro-phenyl)-2-methyl-butyric acid(intermediate 11)and Methyl-(4-trifluoromethyl-benzyl)-amine(commerciallyavailable) | 572.2 | 0.0051 |
| 107 | 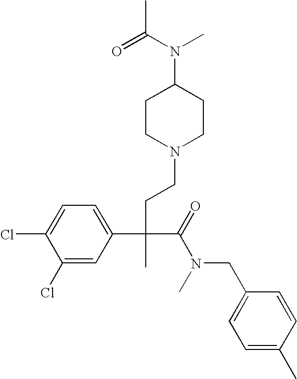 | 518.5 | 4-[4-(Acetyl-methyl-amino)-piperidin-1-yl]-2-(3,4-dichloro-phenyl)-2,N-dimethyl-N-(4-methyl-benzyl)-butyramide | 4-[4-(Acetyl-methyl-amino)-piperidin-1-yl]-2-(3,4-dichloro-phenyl)-2-methyl-butyric acid(intermediate 11)and Methyl-(4-methyl-benzyl)-amine(commerciallyavailable) | 518.4 | 0.0106 |
| 108 | 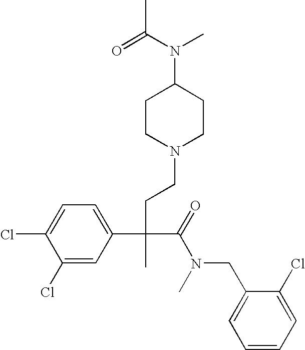 | 539.0 | 4-[4-(Acetyl-methyl-amino)-piperidin-1-yl]-N-(2-chloro-benzyl)-2-(3,4-dichloro-phenyl)-2,N-dimethyl-butyramide | 4-[4-(Acetyl-methyl-amino)-piperidin-1-yl]-2-(3,4-dichloro-phenyl)-2-methyl-butyric acid(intermediate 11)and (2-Chloro-benzyl)-methyl-amine(commerciallyavailable) | 538.5 | 0.012 |
| 109 | 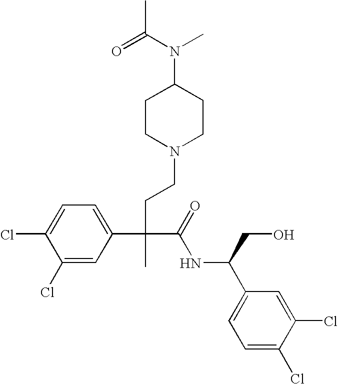 | 589.4 | 4-[4-(Acetyl-methyl-amino)-piperidin-1-yl]-2-(3,4-dichloro-phenyl)-N-[1-(3,4-dichloro-phenyl)-2-hydroxy-ethyl]-2-methyl-butyramide;diastereoisomer 1 | 4-[4-(Acetyl-methyl-amino)-piperidin-1-yl]-2-(3,4-dichloro-phenyl)-2-methyl-butyric acid(intermediate 11)and 2-Amino-2-(3,4-dichloro-phenyl)-ethanol(WO 2005058892) | 590.2 | 0.0056 |
| 110 | 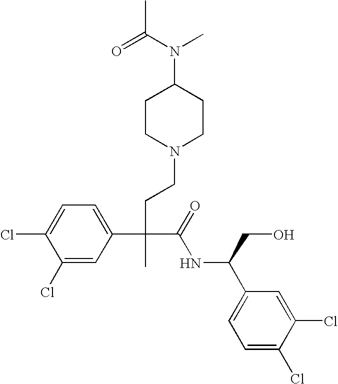 | 589.4 | 4-[4-(Acetyl-methyl-amino)-piperidin-1-yl]-2-(3,4-dichloro-phenyl)-N-[1-(3,4-dichloro-phenyl)-2-hydroxy-ethyl]-2-methyl-butyramide;diastereoisomer 2 | 4-[4-(Acetyl-methyl-amino)-piperidin-1-yl]-2-(3,4-dichloro-phenyl)-2-methyl-butyric acid(intermediate 11)and 2-Amino-2-(3,4-dichloro-phenyl)-ethanol(WO 2005058892) | 590.2 | 0.1103 |

### Example 111

### N-(4-Chloro-benzyl)-2-(4-fluoro-phenyl)-N-methyl-4-[4-(2-oxo-pyrrolidin-1-yl)-piperidin-1-yl]-butyramide

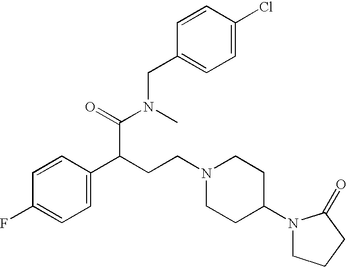

### a) Step 1:

### N-(4-Chloro-benzyl)-2-(4-fluoro-phenyl)-N-methyl-acetamide

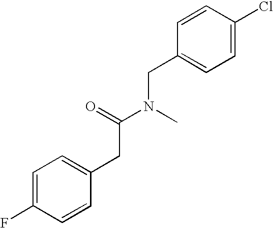

A mixture of 1.09 g (7 mmol) 4-fluorophenylacetic acid, 1.37 g (9 mmol) (4-chloro-benzyl)-methyl-amine, 2.84 g (9 mmol) TBTU and 6 mL (35 mmol) DIPEA in 50 mL DMF was stirred at room temperature for 16 h. After evaporation of all volatiles Na₂CO₃ aq. was added and the mixture was extracted with ethyl acetate. The combined organic phases were washed with NaCl aq., dried with Na₂SO₄, filtered and evaporated to dryness. The residue was purified by column chromatography on silica eluting with a gradient formed from ethyl acetate and heptane. The product containing fractions were evaporated to yield 1.35 g (65%) of the title compound as yellow oil. MS(m/e): 292.2 (MH⁺).

### b) Step 2:

### N-(4-Chloro-benzyl)-2-(4-fluoro-phenyl)-N-methyl-4-oxo-butyramide

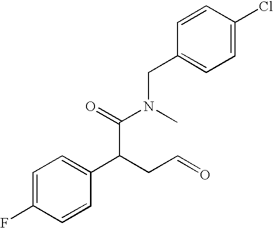

A solution of 1.10 g (3.78 mmol) N-(4-chloro-benzyl)-2-(4-fluoro-phenyl)-N-methyl-acetamide in 10 mL DMF at 0° C. was treated with 0.18 g (4 mmol) NaH (55% in oil) and allowed to stir at 40-50° C. for 3 h. After cooling to 0° C. the mixture was treated with 0.80 g (5 mmol) bromoaetaldehyde dimethylacetal in 10 mL DMF and the mixture was stirred at room temperature for 16 h. After evaporation of all volatiles the residue was treated with water and THF and 1N HCl aq. was added. The mixture was stirred at room temperature for 2 h and extracted with ethyl acetate. The combined organic phases were washed with NaCl aq. sat., dried with Na₂SO₄, filtered and evaporated. The residue was purified by column chromatography on silica eluting with a gradient formed from tert.-butylmethyl ether and heptane. The product containing fractions were evaporated to yield 0.90 g of the title compound which was used without further purification in the consecutive step. MS(m/e): 332.4 (MH⁺).

### c) Step 3:

A mixture of 237 mg (0.71 mmol) N-(4-chloro-benzyl)-2-(4-fluoro-phenyl)-N-methyl-4-oxo-butyramide, 131 mg (0.77 mmol) 1-piperidin-4-yl-pyrrolidin-2-one, 0.226 (10.7 mmol) sodium triacetoxyborohydride and 64 mg acetic acid in 10 mL THF was stirred at room temperature for 17 h. Water and Na₂CO₃ aq. was added and the mixture was extracted with ethyl acetate. The combined organic phases were washed with NaCl aq. sat., dried with Na₂SO₄, filtered and evaporated. The residue was purified by column chromatography on silica eluting with a gradient formed from DCM, methanol and NH₃ aq. The product containing fractions were evaporated to yield 277 mg (0.57 mmol) of the title compound as white foam. MS(m/e): 486.4 (MH⁺).

In analogy to the procedure described for the synthesis of N-(4-chloro-benzyl)-2-(4-fluoro-phenyl)-N-methyl-4-[4-(2-oxo-pyrrolidin-1-yl)-piperidin-1-yl]-butyramide (example 111) further piperidine derivatives have been synthesized from the starting materials listed in table 2. Table 2 comprises example 112 -117.

TABLE 2

| | | | Systematic | | MW | hNK3 |
|---|---|---|---|---|---|---|
| | | | | | found | Ki |
| No | structure | MW | Name | starting materials | (MH+) | (uM) |
| 111 | 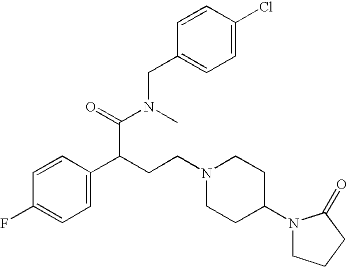 | 486.0 | N-(4-Chloro-benzyl)-2-(4-fluoro-phenyl)-N-methyl-4-[4-(2-oxo-pyrrolidin-1-yl)-piperidin-1-yl]-butyramide | 4-Fluorophenylaceticacid, (4-Chloro-benzyl)-methyl-amine,bromoaetaldehydedimethylacetal and 1-Piperidin-4-yl-pyrrolidin-2-one | 486.4 | 0.4084 |
| 112 | 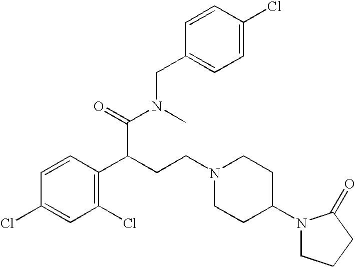 | 536.9 | N-(4-Chloro-benzyl)-2-(2,4-dichloro-phenyl)-N-methyl-4-[4-(2-oxo-pyrrolidin-1-yl)-piperidin-1-yl]-butyramide | 2,4-Dichlorophenylaceticacid, (4-Chloro-benzyl)-methyl-amine,bromoaetaldehydedimethylacetal and 1-Piperidin-4-yl-pyrrolidin-2-one | 536.2 | 0.1281 |
| 113 | 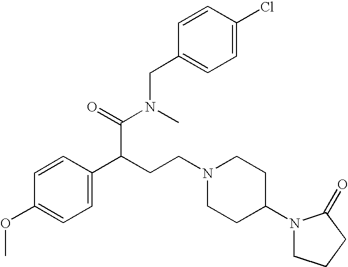 | 498.1 | N-(4-Chloro-benzyl)-2-(4-methoxy-phenyl)-N-methyl-4-[4-(2-oxo-pyrrolidin-1-yl)-piperidin-1-yl]-butyramide | 4-Methoxyphenylaceticacid, (4-Chloro-benzyl)-methyl-amine,bromoaetaldehydedimethylacetal and 1-Piperidin-4-yl-pyrrolidin-2-one | 498.1 | 0.7748 |
| 114 | 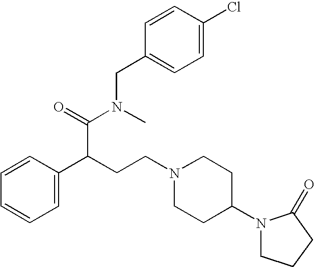 | 468.0 | N-(4-Chloro-benzyl)-N-methyl-4-[4-(2-oxo-pyrrolidin-1-yl)-piperidin-1-yl]-2-phenyl-butyramide | Phenylacetic acid, (4-Chloro-benzyl)-methyl-amine,bromoacetaldehydedimethylacetal and 1-Piperidin-4-yl-pyrrolidin-2-one | 468.3 | 0.5603 |
| 115 | 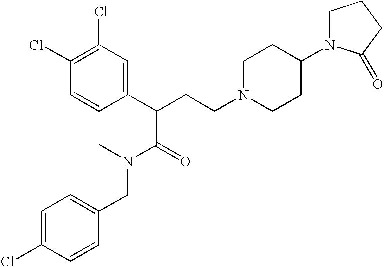 | 536.9 | N-(4-Chloro-benzyl)-2-(3,4-dichloro-phenyl)-N-methyl-4-[4-(2-oxo-pyrrolidin-1-yl)-piperidin-1-yl]-butyramide | 3,4-Dichlorophenylaceticacid, (4-Chloro-benzyl)-methyl-amine,bromoaetaldehydedimethylacetal and 1-Piperidin-4-yl-pyrrolidin-2-one | 536.1 | 0.0081 |
| 116 | 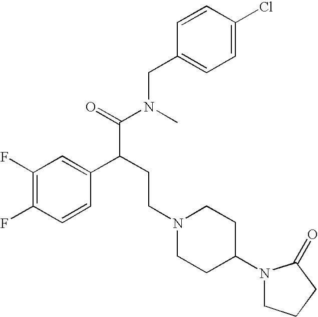 | 504.0 | N-(4-Chloro-benzyl)-2-(3,4-difluoro-phenyl)-N-methyl-4-[4-(2-oxo-pyrrolidin-1-yl)-piperidin-1-yl]-butyramide | 3,4-Difluorophenylaceticacid, (4-Chloro-benzyl)-methyl-amine,bromoaetaldehydedimethylacetal and 1-Piperidin-4-yl-pyrrolidin-2-one | 504.2 | 0.1977 |
| 117 | 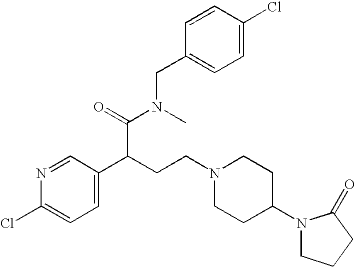 | 503.5 | N-(4-Chloro-benzyl)-2-(6-chloro-pyridin-3-yl)-N-methyl-4-[4-(2-oxo-pyrrolidin-1-yl)-piperidin-1-yl]-butyramide | (6-Chloro-pyridin-3-yl)-acetic acid, (4-Chloro-benzyl)-methyl-amine,bromoaetaldehydedimethylacetal and 1-Piperidin-4-yl-pyrrolidin-2-one | 503.2 | 0.3872 |

Enantiomers were accessed from their respective starting materials (as listed in table 3) by column chromatography on appropriate chiral phase. Isolated compounds were optionally transferred into their respective salts by treatment with acid. Table 3 comprises example 118-124.

TABLE 3

| | | | | | MW | hNK3 |
|---|---|---|---|---|---|---|
| | | | | starting | found | Ki |
| No | structure | MW | Systematic Name | materials | (MH+) | (uM) |
| 118 | 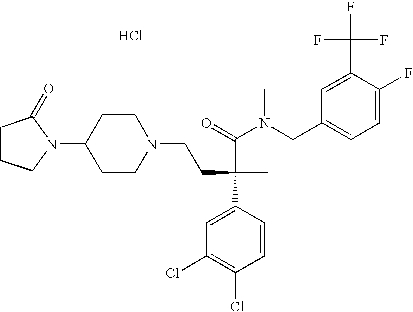 | 639.0 | (S or R)-2-(3,4-Dichloro-phenyl)-N-(4-fluoro-3-trifluoromethyl-benzyl)-2,N-dimethyl-4-[4-(2-oxo-pyrrolidin-1-yl)-piperidin-1-yl]-butyramide;hydrochloride | chiralseparationfromexample 86 | 602.4 | 0.1709 |
| 119 | 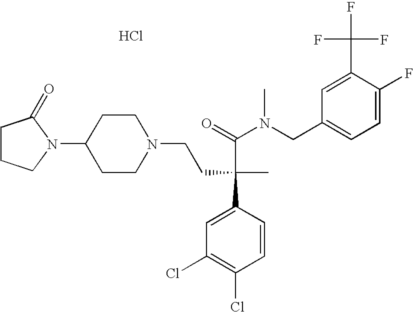 | 639.0 | (R or S)-2-(3,4-Dichloro-phenyl)-N-(4-fluoro-3-trifluoromethyl-benzyl)-2,N-dimethyl-4-[4-(2-oxo-pyrrolidin-1-yl)-piperidin-1-yl]-butyramide;hydrochloride | chiralseparationfromexample 86 | 602.4 | 0.0008 |
| 120 | 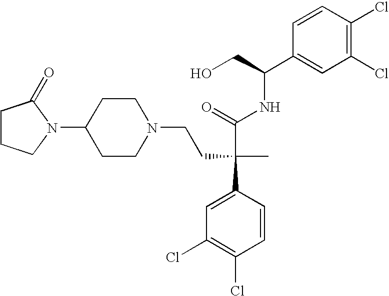 | 601.4 | (R or S)-2-(3,4-Dichloro-phenyl)-N-[(S or R)-1-(3,4-dichloro-phenyl)-2-methyl-4-[4-(2-oxo-pyrrolidin-1-yl)-piperidin-1-yl]-butyramide | chiralseparationfromexample 94 | 602.4 | 0.002 |
| 121 | 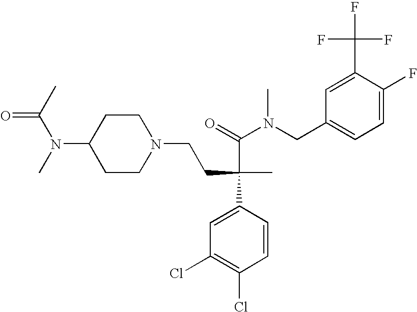 | 590.5 | (S or R)-4-[4-(Acetyl-methyl-amino)-piperidin-1-yl]-2-(3,4-dichloro-phenyl)-N-(4-fluoro-3-trifluoromethyl-benzyl)-2,N-dimethyl-butyramide | chiralseparationfromexample104 | 590.3 | 0.1735 |
| 122 | 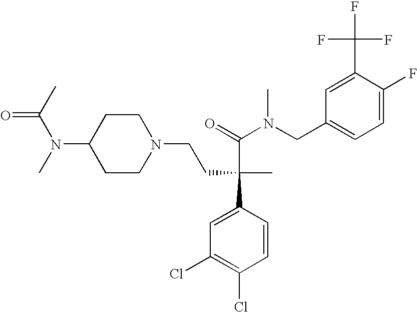 | 590.5 | (R or S)-4-[4-(Acetyl-methyl-amino)-piperidin-1-yl]-2-(3,4-dichloro-phenyl)-N-(4-fluoro-3-trifluoromethyl-benzyl)-2,N-dimethyl-butyramide | chiralseparationfromexample104 | 590.3 | 0.0015 |
| 123 | 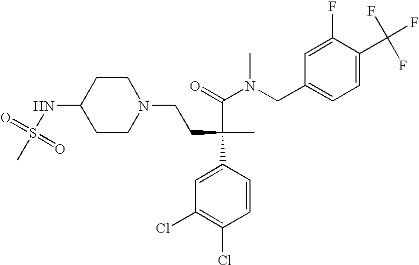 | 612.5 | (S or R)-2-(3,4-Dichloro-phenyl)-N-(3-fluoro-4-trifluoromethyl-benzyl)-4-(4-methanesulfonylamino-piperidin-1-yl)-2,N-dimethyl-butyramide | chiralseparationfromexample 45 | 612.2 | 0.8004 |
| 124 | 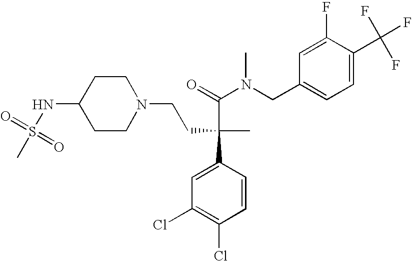 | 612.5 | (R or S)-2-(3,4-Dichloro-phenyl)-N-(3-fluoro-4-trifluoromethyl-benzyl)-4-(4-methanesulfonylamino-piperidin-1-yl)-2,N-dimethyl-butyramide | chiralseparationfromexample 45 | 612.2 | 0.0188 |

### Example 125

### 2-(3,4-Dichloro-phenyl)-N-[-(4-fluoro-3 -trifluoromethyl-phenyl)-ethyl]-2-methyl-4-[4-(2-oxo-pyrrolidin-1-yl)-piperidin-1-yl]-butyramide

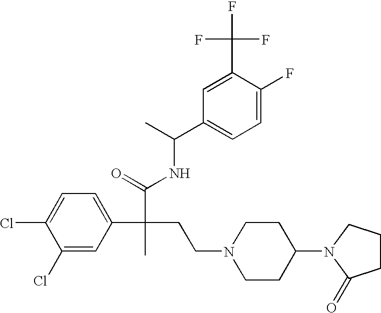

A mixture of 0.1 g (0.22 mmol) 2-(3,4-dichloro-phenyl)-2-methyl-4-[4-(2-oxo-pyrrolidin-1-yl)-piperidin-1-yl]-butyric acid (intermediate 10), 0.068 g (0.027 mmol) 1-(4-Fluoro-3-trifluoromethyl-phenyl)-ethylamine; hydrochloride (intermediate 20), 0.016 g (0.027 mmol) HATU and 0.23 mL DIPEA in 1.5 mL DMF was stirred at room temperature over night. The mixture was subjected to purification by reversed phase HPLC eluting with a gradient formed from acetonitrile, water and NEt3. the product containing fractions were evaporated to yield 0.079 g (59%) of the title compound as light yellow viscous oil. MS(m/e): 601.9 (MH⁺).

In analogy to the procedure described for the synthesis of2-(3,4-dichloro-phenyl)-N-[1-(4-fluoro-3-trifluoromethyl-phenyl)-ethyl]-2-methyl-4-[4-(2-oxo-pyrrolidin-1-yl)-piperidin-1-yl]-butyramide (example 125) further piperidine derivatives have been prepared from the starting materials listed in table 4. Optionally diastereoisomeric/epimeric mixture have been separated by column chromatography. Table 4 comprises example 125-example 169.

TABLE 4

| | | | | | MW | |
|---|---|---|---|---|---|---|
| | | | | | found | hNK3 |
| No | structure | MW | Systematic Name | starting materials | (MH+) | Ki(uM) |
| 125 | 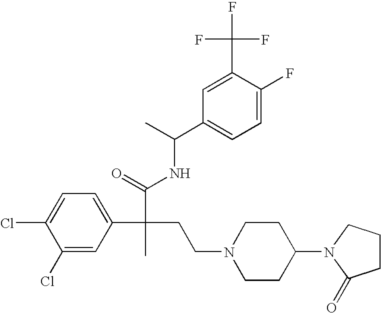 | 602.497 | 2-(3,4-Dichloro-phenyl)-N-[1-(4-fluoro-3-trifluoromethyl-phenyl)-ethyl]-2-methyl-4-[4-(2-oxo-pyrrolidin-1-yl)-piperidin-1-yl]-butyramide | 2-(3,4-Dichloro-phenyl)-2-methyl-4-[4-(2-oxo-pyrrolidin-1-yl)-piperidin-1-yl]-butyric acid(intermediate 10) and1-(4-Fluoro-3-trifluoromethyl-phenyl)-ethylamine;hydrochloride(intermediate 19) | 601.9 | 0.0254 |
| 126 | 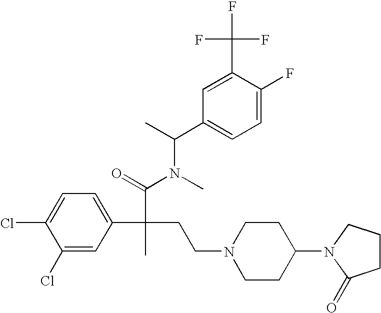 | 616.523 | 2-(3,4-Dichloro-phenyl)-N-[1-(4-fluoro-3-trifluoromethyl-phenyl)-ethyl]-2,N-dimethyl-4-[4-(2-oxo-pyrrolidin-1-yl)-piperidin-1-yl]-butyramide | 2-(3,4-Dichloro-phenyl)-2-methyl-4-[4-(2-oxo-pyrrolidin-1-yl)-piperidin-1-yl]-butyric acid(intermediate 10) and1-(4-Fluoro-3-trifluoromethyl-phenyl)-ethyl-methylamine; hydrochloride(Intermediate 20) | 615.9 | 0.0303 |
| 127 | 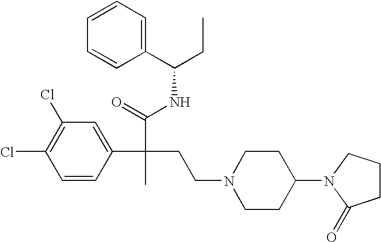 | 530.536 | 2-(3,4-Dichloro-phenyl)-2-methyl-4-[4-(2-oxo-pyrrolidin-1-yl)-piperidin-1-yl]-N-((S)-1-phenyl-propyl)-butyramide | 2-(3,4-Dichloro-phenyl)-2-methyl-4-[4-(2-oxo-pyrrolidin-1-yl)-piperidin-1-yl]-butyric acid(intermediate 10) and(S)-(−)-1-phenylpropylamine(commerciallyavailable) | 530.2 | 0.0052 |
| 128 | 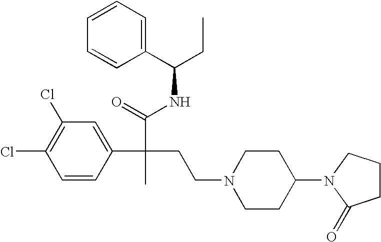 | 530.536 | 2-(3,4-Dichloro-phenyl)-2-methyl-4-[4-(2-oxo-pyrrolidin-1-yl)-piperidin-1-yl]-N-((R)-1-phenyl-propyl)-butyramide | 2-(3,4-Dichloro-phenyl)-2-methyl-4-[4-(2-oxo-pyrrolidin-1-yl)-piperidin-1-yl]-butyric acid(intermediate 10) and(R)-(+)-1-phenylpropylamine(commerciallyavailable) | 530.2 | 0.0902 |
| 129 | 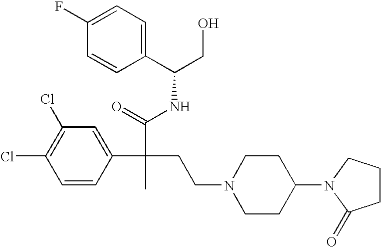 | 550.499 | 2-(3,4-Dichloro-phenyl)-N-[(R)-1-(4-fluoro-phenyl)-2-hydroxy-ethyl]-2-(R or S) methyl-4-[4-(2-oxo-pyrrolidin-1-yl)-piperidin-1-yl]-butyramide | 2-(3,4-Dichloro-phenyl)-2-methyl-4-[4-(2-oxo-pyrrolidin-1-yl)-piperidin-1-yl]-butyric acid(intermediate 10) and(R)-2-Amino-2-(4-fluoro-phenyl)-ethanol(commerciallyavailable) | 550.4 | 0.0082 |
| 130 | 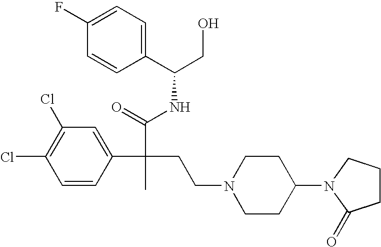 | 550.499 | 2-(3,4-Dichloro-phenyl)-N-[(R)-1-(4-fluoro-phenyl)-2-hydroxy-ethyl]-2-(S or R) methy-4-[4-(2-oxo-pyrrolidin-1-yl)-piperidin-1-yl]-butyramide | 2-(3,4-Dichloro-phenyl)-2-methyl-4-[4-(2-oxo-pyrrolidin-1-yl)-piperidin-1-yl]-butyric acid(intermediate 10) | 550.4 | 0.1007 |
| 131 | 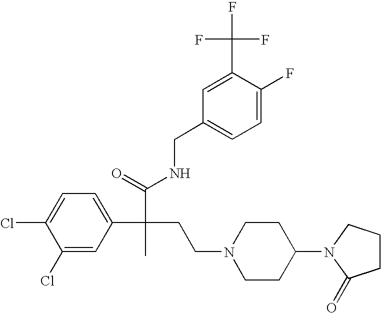 | 588.47 | 2-(3,4-Dichloro-phenyl)-N-(4-fluoro-3-trifluoromethyl-benzyl)-2-methyl-4-[4-(2-oxo-pyrrolidin-1-yl)-piperidin-1-yl]-butyramide | 2-(3,4-Dichloro-phenyl)-2-methyl-4-[4-(2-oxo-pyrrolidin-1-yl)-piperidin-1-yl]-butyric acid(intermediate 10) and4-fluoro-3-(trifluoro-methyl)benzyl-amine(commerciallyavailable | 588.1 | 0.0034 |
| 132 | 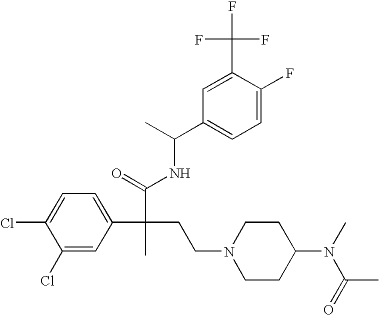 | 590.486 | 4-[4-(Acetyl-methyl-amino)-piperidin-1-yl]-2-(3,4-dichloro-phenyl)-N-[1-(4-fluoro-3-trifluoromethyl-phenyl)-ethyl]-2-methyl-butyramide | 4-[4-(Acetyl-methyl-amino)-piperidin-1-yl]-2-(3,4-dichloro-phenyl)-2-methyl-butyric acid(intermediate 11) and1-(4-Fluoro-3-trifluoromethyl-phenyl)-ethylamine;hydrochloride(intermediate 19) | 589.9 | 0.0462 |
| 133 | 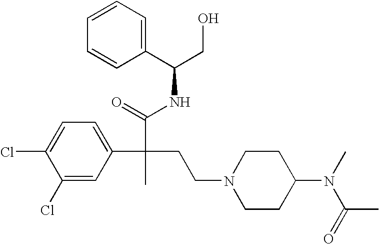 | 520.497 | 4-[4-(Acetyl-methyl-amino)-piperidin-1-yl]-2-(3,4-dichloro-phenyl)-N-((S)-2-hydroxy-1-phenyl-ethyl)-2-methyl-butyramide | 4-[4-(Acetyl-methyl-amino)-piperidin-1-yl]-2-(3,4-dichloro-phenyl)-2-methyl-butyric acid(intermediate 11) and(S)-(−)-1-phenylpropylamine(commerciallyavailable) | 520.2 | 0.5257 |
| 134 | 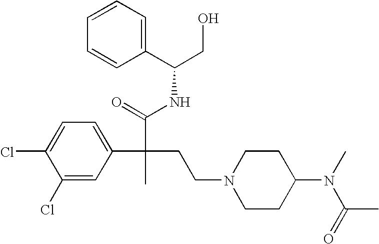 | 520.497 | 4-[4-(Acetyl-methyl-amino)-piperidin-1-yl]-2-(3,4-dichloro-phenyl)-N-((R)-2-hydroxy-1-phenyl-ethyl)-2-methyl-butyramide | 4-[4-(Acetyl-methyl-amino)-piperidin-1-yl]-2-(3,4-dichloro-phenyl)-2-methyl-butyric acid(intermediate 11) and(R)-(−)-1-phenylpropylamine(commerciallyavailable) | 520.3 | 0.0356 |
| 135 | 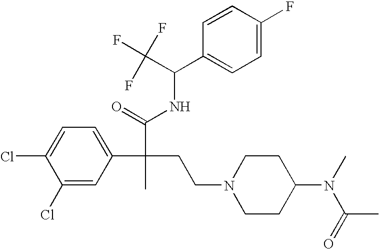 | 576.459 | 4-[4-(Acetyl-methyl-amino)-piperidin-1-yl]-2-(3,4-dichloro-phenyl)-2-methyl-N-[2,2,2-trifluoro-1-(4-fluoro-phenyl)-ethyl]-butyramide | 4-[4-(Acetyl-methyl-amino)-piperidin-1-yl]-2-(3,4-dichloro-phenyl)-2-methyl-butyric acid(intermediate 11) and2,2,2-Trifluoro-1-(4-fluoro-phenyl)ethylamine(commerciallyavailable) | 575.8 | 0.0126 |
| 136 | 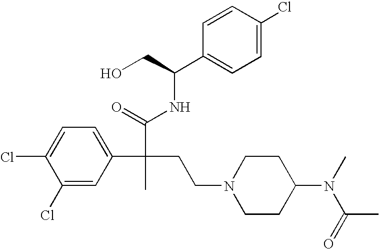 | 554.943 | 4-[4-(Acetyl-methyl-amino)-piperidin-1-yl]-N-[1-(4-chloro-phenyl)-2-hydroxy-ethyl]-2-(3,4-dichloro-phenyl)-2-methyl-butyramide(diastereosisomer1) | 4-[4-(Acetyl-methyl-amino)-piperidin-1-yl]-2-(3,4-dichloro-phenyl)-2-methyl-butyric acid(intermediate 11) and4-Chlorophenylglycinol(commerciallyavailable) | 553.7 | 0.0096 |
| 137 | 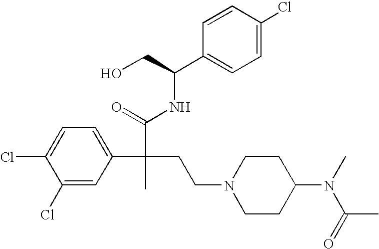 | 554.943 | 4-[4-(Acetyl-methyl-amino)-piperidin-1-yl]-N-[1-(4-chloro-phenyl)-2-hydroxy-ethyl]-2-(3,4-dichloro-phenyl)-2-methyl-butyramide(diastereosisomer2) | 4-[4-(Acetyl-methyl-amino)-piperidin-1-yl]-2-(3,4-dichloro-phenyl)-2-methyl-butyric acid(intermediate 11) and4-Chlorophenylglycinol(commerciallyavailable) | 553.7 | 0.056 |
| 138 | 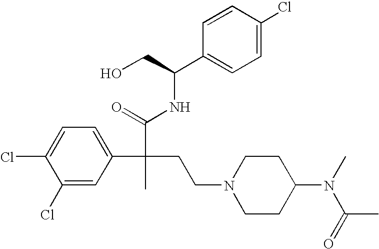 | 538.488 | 4-[4-(Acetyl-methyl-amino)-piperidin-1-yl]-2-(3,4-dichloro-phenyl)-N-[1-(4-fluoro-phenyl)-2-hydroxy-ethyl]-2-methyl-butyramide(diastereoisomer 1) | 4-[4-(Acetyl-methyl-amino)-piperidin-1-yl]-2-(3,4-dichloro-phenyl)-2-methyl-butyric acid(intermediate 11) and4-Fluorophenylglycinol(commerciallyavailable) | 538 | 0.0293 |
| 139 | 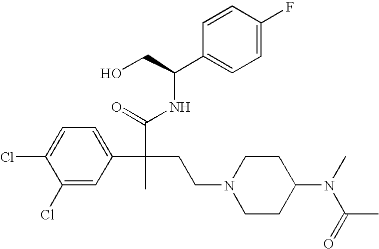 | 538.488 | 4-[4-(Acetyl-methyl-amino)-piperidin-1-yl]-2-(3,4-dichloro-phenyl)-N-[1-(4-fluoro-phenyl)-2-hydroxy-ethyl]-2-methyl-butyramide(diastereoisomer 2) | 4-[4-(Acetyl-methyl-amino)-piperidin-1-yl]-2-(3,4-dichloro-phenyl)-2-methyl-butyric acid(intermediate 11) and4-Fluorophenylglycinol(commerciallyavailable) | 538 | 0.1415 |
| 140 | 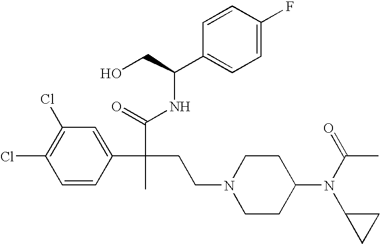 | 564.53 | 4-[4-(Acetyl-cyclopropyl-amino)-piperidin-1-yl]-2-(3,4-dichloro-phenyl)-N-[1-(4-fluoro-phenyl)-2-hydroxy-ethyl]-2-methyl-butyramide(diastereoisomer 1) | 4-[4-(Acetyl-cyclopropyl-amino)-piperidin-1-yl]-2-(3,4-dichloro-phenyl)-2-methyl-butyric acid(intermediate 14) and4-Fluorophenylglycinol(commerciallyavailable) | 564.4 | 0.0333 |
| 141 | 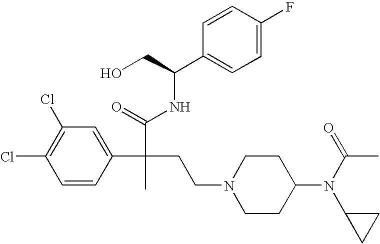 | 564.53 | 4-[4-(Acetyl-cyclopropyl-amino)-piperidin-1-yl]-2-(3,4-dichloro-phenyl)-N-[1-(4-fluoro-phenyl)-2-hydroxy-ethyl]-2-methyl-butyramide(diastereoisomer 2) | 4-[4-(Acetyl-cylcopropyl-amino)-piperidin-1-yl]-2-(3,4-dichloro-phenyl)-2-methyl-butyric acid(intermediate 14) and4-Fluorophenylglycinol(commerciallyavailable) | 564.4 | 0.1648 |
| 142 | 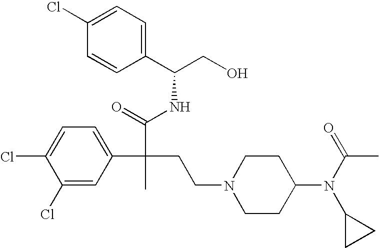 | 580.98 | 4-[4-(Acetyl-cyclopropyl-amino)-piperidin-1-yl]-N-[1-(4-chloro-phenyl)-2-hydroxy-ethyl]-2-(3,4-dichloro-phenyl)-2-methyl-butyramide(diastereoisomer 1) | 4-[4-(Acetyl-cyclopropyl-amino)-piperidin-1-yl]-2-(3,4-dichloro-phenyl)-2-methyl-butyric acid(intermediate 14) and4-Chlorophenylglycinol(commerciallyavailable) | 580.4 | 0.0109 |
| 143 | 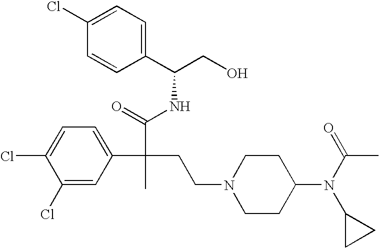 | 580.98 | 4-[4-(Acetyl-cyclopropyl-amino)-piperidin-1-yl]-N-[1-(4-chloro-phenyl)-2-hydroxy-ethyl]-2-(3,4-dichloro-phenyl)-2-methyl-butyramide(diastereoisomer 2) | 4-[4-(Acetyl-cyclopropyl-amino)-piperidin-1-yl]-2-(3,4-dichloro-phenyl)-2-methyl-butyric acid(intermediate 14) and4-Chlorophenylglycinol(commerciallyavailable) | 580.4 | 0.0877 |
| 144 | 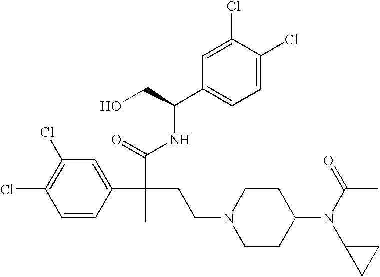 | 615.43 | 4-[4-(Acetyl-cylcopropyl-amino)-piperidin-1-yl]-2-(3,4-dichloro-phenyl)-N-[1-(3,4-dichloro-phenyl)-2-hydroxy-ethyl]-2-methyl-butyramide(diastereosiomer 1) | 4-[4-(Acetyl-cyclopropyl-amino)-piperidin-1-yl]-2-(3,4-dichloro-phenyl)-2-methyl-butyric acid(intermediate 14) and3,4-di-Chlorophenylglycinol(commerciallyavailable) | 616.2 | 0.0101 |
| 145 | 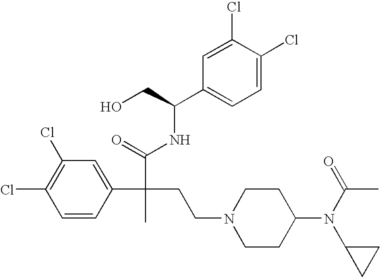 | 615.43 | 4-[4-(Acetyl-cyclopropyl-amino)-piperidin-1-yl]-2-(3,4-dichloro-phenyl)-N-[1-(3,4-dichloro-phenyl)-2-hydroxy-ethyl]-2-methyl-butyramide(diastereoisomer 2) | 4-[4-(Acetyl-cyclopropyl-amino)-piperidin-1-yl]-2-(3,4-dichloro-phenyl)-2-methyl-butyric acid(intermediate 14) and3,4-di-Chlorophenylglycinol(commerciallyavailable) | 616.2 | 0.0502 |
| 146 | 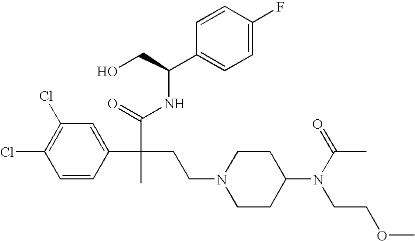 | 582.54 | 4-{4-[Acetyl-(2-methoxy-ethyl)-amino]-piperidin-1-yl}-2-(3,4-dichloro-phenyl)-N-[1-(4-fluoro-phenyl)-2-hydroxy-ethyl]-2-methyl-butyramidediastereoisomer 1) | 4-{4-[Acetyl-(2-methoxy-ethyl)-amino]-piperidin-1-yl}-2-(3,4-dichloro-phenyl)-2-methyl-butyric acid(intermediate 15) and4-Fluorophenylglycinol(commerciallyavailable) | 582.2 | 0.0007 |
| 147 | 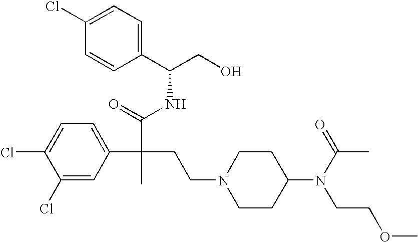 | 598.995 | 4-{4-[Acetyl-(2-methoxy-ethyl)-amino]-piperidin-1-yl}-N-[1-(4-chloro-phenyl)-2-hydroxy-ethyl]-2-(3,4-dichloro-phenyl)-2-methyl-butyramide | 4-{4-[Acetyl-(2-methoxy-ethyl)-amino]-piperidin-1-yl}-2-(3,4-dichloro-phenyl)-2-methyl-butyric acid(intermediate 15) and4-Chlorophenylglycinol(commerciallyavailable) | 598.3 | 0.3824 |
| 148 | 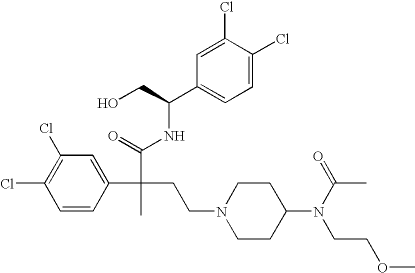 | 633.44 | 4-{4-[Acetyl-(2-methoxy-ethyl)-amino]-piperidin-1-yl}-2-(3,4-dichloro-phenyl)-N-[1-(3,4-dichloro-phenyl)-2-hydroxy-ethyl]-2-methyl-butyramide(diastreoisomer 1) | 4-{4-[Acetyl-(2-methoxy-ethyl)-amino]-piperidin-1-yl}-2-(3,4-dichloro-phenyl)-2-methyl-butyric acid(intermediate 15) and3,4-di-Chlorophenylglycinol(commerciallyavailable) | 634.2 | 0.0038 |
| 149 | 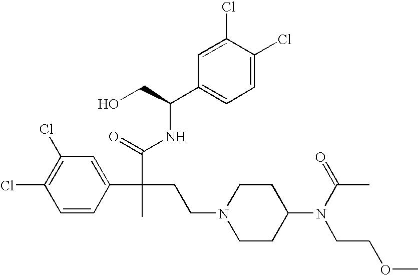 | 633.44 | 4-{4-[Acetyl-(2-methoxy-ethyl)-amino]-piperidin-1-yl}-2-(3,4-dichloro-phenyl)-N-[(S)-1-(3,4-dichloro-phenyl)-2-hydroxy-ethyl]-2-methyl-butyramide(diastreoisomer 2) | 4-{4-[Acetyl-(2-methoxy-ethyl)-amino]-piperidin-1-yl}-2-(3,4-dichloro-phenyl)-2-methyl-butyric acid(intermediate 15) and3,4-di-Chlorophenylglycinol(commerciallyavailable) | 634.3 | 0.0194 |
| 150 | 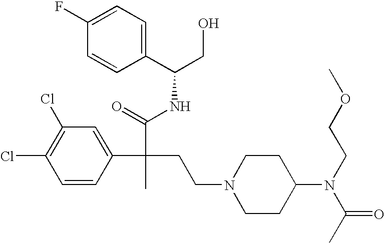 | 582.54 | 4-{4-[Acetyl-(2-methoxy-ethyl)-amino]-piperidin-1-yl}-2-(3,4-dichloro-phenyl)-N-[(R)-1-(4-fluoro-phenyl)-2-hydroxy-ethyl]-2-(R or S)methyl-butyramide | 4-{4-[Acetyl-(2-methoxy-ethyl)-amino]-piperidin-1-yl}-2-(3,4-dichloro-phenyl)-2-methyl-butyric acid(intermediate 15) and(R)-4-Fluorophenylglycinol(commerciallyavailable) | 582.2 | 0.0017 |
| 151 | 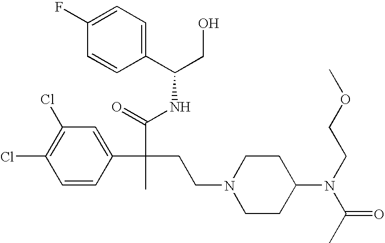 | 582.54 | 4-{4-[Acetyl-(2-methoxy-ethyl)-amino]-piperidin-1-yl}-2-(3,4-dichloro-phenyl)-N-[(R)-1-(4-fluoro-phenyl)-2-hydroxy-ethyl]-2-(S or R)methyl-butyramide | 4-{4-[Acetyl-(2-methoxy-ethyl)-amino]-piperidin-1-yl}-2-(3,4-dichloro-phenyl)-2-methyl-butyric acid(intermediate 15) and(R)-4-Fluorophenylglycinol(commerciallyavailable) | 582.2 | 0.0211 |
| 152 | 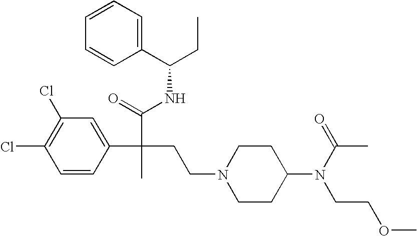 | 562.578 | 4-{4-[Acetyl-(2-methoxy-ethyl)-amino]-piperidin-1-yl}-2-(3,4-dichloro-phenyl)-2-methyl-N-((S)-1-phenyl-propyl)-butyramide | 4-{4-[Acetyl-(2-methoxy-ethyl)-amino]-piperidin-1-yl}-2-(3,4-dichloro-phenyl)-2-methyl-butyric acid(intermediate 15) and(S)-(−)-1-phenylpropylamine(commerciallyavailable) | 562.2 | 0.0011 |
| 153 | 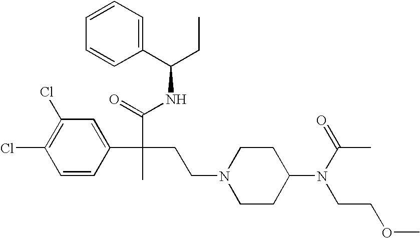 | 562.578 | 4-{4-[Acetyl-(2-methoxy-ethyl)-amino]-piperidin-1-yl}-2-(3,4-dichloro-phenyl)-2-methyl-N-((R)-1-phenyl-propyl)-butyramide | 4-{4-[Acetyl-(2-methoxy-ethyl)-amino]-piperidin-1-yl}-2-(3,4-dichloro-phenyl)-2-methyl-butyric acid(intermediate 15) and(R)-(+)-1-phenylpropylamine(commerciallyavailable) | 562.2 | 0.0609 |
| 154 | 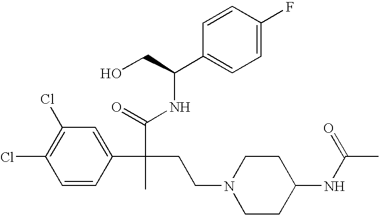 | 524.46 | 4-(4-Acetylamino-piperidin-1-yl)-2-(3,4-dichloro-phenyl)-N-[1-(4-fluoro-phenyl)-2-hydroxy-ethyl]-2-methyl-butyramide(diastereoisomer 1) | 4-(4-Acetylamino-piperidin-1-yl)-2-(3,4-dichloro-phenyl)-2-methylbutyric acid(intermediate 16) and4-Fluorophenylglycinol(commerciallyavailable) | 524.3 | 0.1703 |
| 155 | 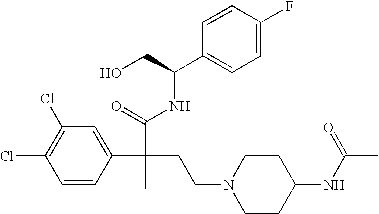 | 524.46 | 4-(4-Acetylamino-piperidin-1-yl)-2-(3,4-dichloro-phenyl)-N-[1-(4-fluoro-phenyl)-2-hydroxy-ethyl]-2-methyl-butyramide(diastereoisomer 2) | 4-(4-Acetylamino-piperidin-1-yl)-2-(3,4-dichloro-phenyl)-2-methylbutyric acid(intermediate 16) and4-Fluorophenylglycinol(commerciallyavailable) | 524.3 | 0.0138 |
| 156 | 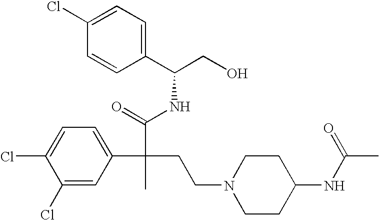 | 540.916 | 4-(4-Acetylamino-piperidin-1-yl)-N-[1-(4-chloro-phenyl)-2-hydroxy-ethyl]-2-(3,4-dichloro-phenyl)-2-methyl-butyramide(diastereoisomer 1) | 4-(4-Acetylamino-piperidin-1-yl)-2-(3,4-dichloro-phenyl)-2-methylbutyric acid(intermediate 16) and4-Chlorophenylglycinol(commerciallyavailable) | 540.3 | 0.0505 |
| 157 | 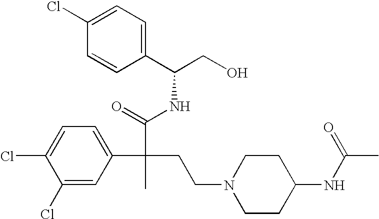 | 540.916 | 4-(4-Acetylamino-piperidin-1-yl)-N-[1-(4-chloro-phenyl)-2-hydroxy-ethyl]-2-(3,4-dichloro-phenyl)-2-methyl-butyramide(diastereoisomer 2) | 4-(4-Acetylamino-piperidin-1-yl)-2-(3,4-dichloro-phenyl)-2-methylbutyric acid(intermediate 16) and4-Chlorophenylglycinol(commerciallyavailable) | 540.3 | 0.7727 |
| 158 | 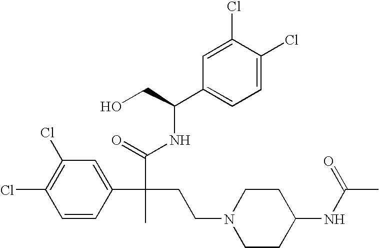 | 575.36 | 4-(4-Acetylamino-piperidin-1-yl)-2-(3,4-dichloro-phenyl)-N-[(R)-1-(3,4-dichloro-phenyl)-2-hydroxy-ethyl]-2-methyl-butyramide(diastereoisomer 1) | 4-(4-Acetylamino-piperidin-1-yl)-2-(3,4-dichloro-phenyl)-2-methylbutyric acid(intermediate 16) and3,4-di-Chlorophenylglycinol(commerciallyavailable) | 576.3 | 0.0346 |
| 159 | 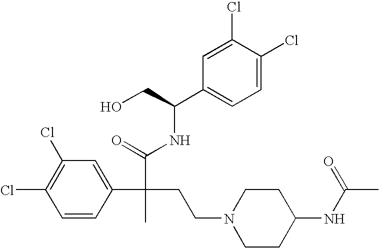 | 575.36 | 4-(4-Acetylamino-piperidin-1-yl)-2-(3,4-dichloro-phenyl)-N-[(S)-1-(3,4-dichloro-phenyl)-2-hydroxy-ethyl]-2-methyl-butyramide(diastereoisomer 2) | 4-(4-Acetylamino-piperidin-1-yl)-2-(3,4-dichloro-phenyl)-2-methylbutyric acid(intermediate 16) and3,4-di-Chlorophenylglycinol(commerciallyavailable) | 576.3 | 0.2582 |
| 160 | 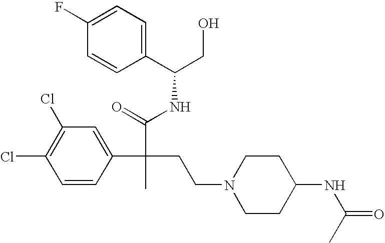 | 524.461 | 4-(4-Acetylamino-piperidin-1-yl)-2-(3,4-dichloro-phenyl)-N-[(R)-1-(4-fluoro-phenyl)-2-hydroxy-ethyl]-2-methyl-butyramide | 4-(4-Acetylamino-piperidin-1-yl)-2-(3,4-dichloro-phenyl)-2-methylbutyric acid(intermediate 16) and(R)-4-Fluorophenylglycinol(commerciallyavailable) | 524.4 | 0.1998 |
| 161 | 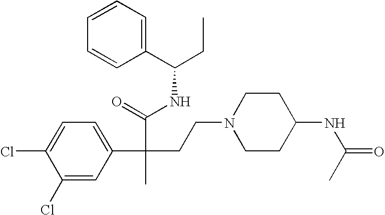 | 504.498 | 4-(4-Acetylamino-piperidin-1-yl)-2-(3,4-dichloro-phenyl)-2-methyl-N-((S)-1-phenyl-propyl)-butyramide | 4-(4-Acetylamino-piperidin-1-yl)-2-(3,4-dichloro-phenyl)-2-methylbutyric acid(intermediate 16) and(S)-(−)-1-phenylpropylamine(commerciallyavailable) | 504.2 | 0.0442 |
| 162 | 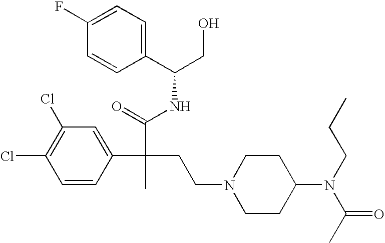 | 566.541 | 4-[4-(Acetyl-propyl-amino)-piperidin-1-yl]-2-(3,4-dichloro-phenyl)-N-[(R)-1-(4-fluoro-phenyl)-2-hydroxy-ethyl]-2-methyl-butyramide(epimer 1) | 4-[4-(Acetyl-propyl-amino)-piperidin-1-yl]-2-(3,4-dichloro-phenyl)-2-methyl-butyric acid(intermediate 17) and(R)-4-Fluorophenylglycinol(commerciallyavailable) | 566.4 | 0.0005 |
| 163 | 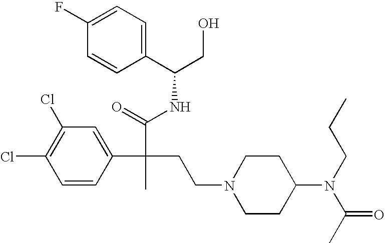 | 566.541 | 4-[4-(Acetyl-propyl-amino)-piperidin-1-yl]-2-(3,4-dichloro-phenyl)-N-[(R)-1-(4-fluoro-phenyl)-2-hydroxy-ethyl]-2-methyl-butyramide(epimer 2) | 4-[4-(Acetyl-propyl-amino)-piperidin-1-yl]-2-(3,4-dichloro-phenyl)-2-methyl-butyric acid(intermediate 17)and (R)-4-Fluorophenylglycinol(commerciallyavailable) | 566.4 | 0.0056 |
| 164 | 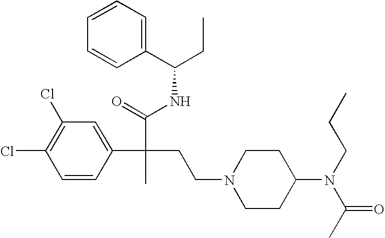 | 546.579 | 4-[4-(Acetyl-propyl-amino)-piperidin-1-yl]-2-(3,4-dichloro-phenyl)-2-methyl-N-((S)-1-phenyl-propyl)-butyramide | 4-[4-(Acetyl-propyl-amino)-piperidin-1-yl]-2-(3,4-dichloro-phenyl)-2-methyl-butyric acid(intermediate 17) and(S)-(−)-1-phenylpropylamine(commerciallyavailable) | 546.2 | 0.0008 |
| 165 | 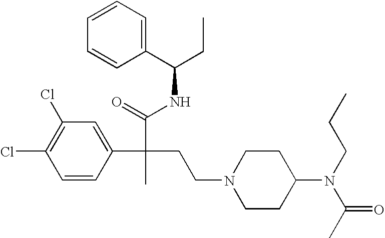 | 546.579 | 4-[4-(Acetyl-propyl-amino)-piperidin-1-yl]-2-(3,4-dichloro-phenyl)-2-methyl-N-((R)-1-phenyl-propyl)-butyramide | 4-[4-(Acetyl-propyl-amino)-piperidin-1-yl]-2-(3,4-dichloro-phenyl)-2-methyl-butyric acid(intermediate 17) and(R)-(+)-1-phenylpropylamine(commerciallyavailable) | 546.2 | 0.0145 |
| 166 | 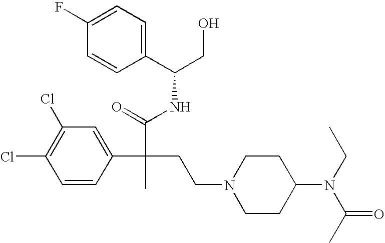 | 552.514 | 4-[4-(Acetyl-ethyl-amino)-piperidin-1-yl]-2-(3,4-dichloro-phenyl)-N-[(R)-1-(4-fluoro-phenyl)-2-hydroxy-ethyl]-2-methyl-butyramide (epimer1) | 4-[4-(Acetyl-ethyl-amino)-piperidin-1-yl]-2-(3,4-dichloro-phenyl)-2-methyl-butyric acid(intermediate 18) and(R)-4-Fluorophenylgycinol(commerciallyavailable) | 552.4 | 0.0022 |
| 167 | 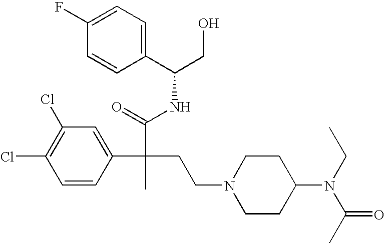 | 552.514 | 4-[4-(Acetyl-ethyl-amino)-piperidin-1-yl]-2-(3,4-dichloro-phenyl)-N-[(R)-1-(4-fluoro-phenyl)-2-hydroxy-ethyl]-2-methyl-butyramide (epimer2) | 4-[4-(Acetyl-ethyl-amino)-piperidin-1-yl]-2-(3,4-dichloro-phenyl)-2-methyl-butyric acid(intermediate 18) and(R)-4-Fluorophenylglycinol(commerciallyavailable) | 552.4 | 0.0349 |
| 168 | 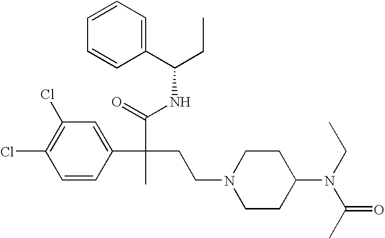 | 532.552 | 4-[4-(Acetyl-ethyl-amino)-piperidin-1-yl]-2-(3,4-dichloro-phenyl)-2-methyl-N-((S)-1-phenyl-propyl)-butyramide | 4-[4-(Acetyl-ethyl-amino)-piperidin-1-yl]-2-(3,4-dichloro-phenyl)-2-methyl-butyric acid(intermediate 18) and(S)-(−)-1-phenylpropylamine(commerciallyavailable) | 532.2 | 0.003 |
| 169 | 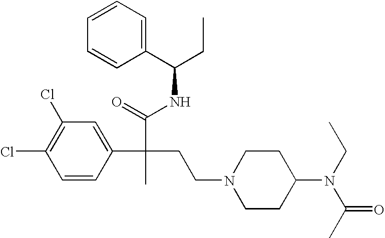 | 532.552 | 4-[4-(Acetyl-ethyl-amino)-piperidin-1-yl]-2-(3,4-dichloro-phenyl)-2-methyl-N-((R)-1-phenyl-propyl)-butyramide | 4-[4-(Acetyl-ethyl-amino)-piperidin-1-yl]-2-(3,4-dichloro-phenyl)-2-methyl-butyric acid(intermediate 18) and(R)-(+)-1-phenylpropylamine(commerciallyavailable) | 532.3 | 0.0576 |

The salt formation is effected at room temperature in accordance with methods which are known per se and which are familiar to any person skilled in the art. Not only salts with inorganic acids, but also salts with organic acids come into consideration. Hydrochlorides, hydrobromides, sulphates, nitrates, citrates, acetates, maleates, succinates, methan-sulphonates, p-toluenesulphonates and the like are examples of such salts.

As mentioned earlier, the compounds of formula I and their pharmaceutically usable addition salts possess valuable pharmacological properties. The compounds of the present invention are antagonists of neurokinin 3 (NK-3) receptors. The compounds were investigated in accordance with the tests given hereinafter.

### [³H]SR142801 Competition Binding Assay

hNK3 receptor binding experiment were performed using [³H]SR142801 (Catalog No. TRK1035, specific activity: 74.0 Ci/mmol, Amersham, GE Healthcare UK limited, Buckinghamshire, UK) and membrane isolated from HEK293 cells transiently expressing recombinant human NK3 receptor. After thawing, the membrane homogenates were centrifuged at 48,000×g for 10 min at 4° C., the pellets were resuspended in the 50 mM Tris-HCl, 4 mM MnCl₂, 1 μM phosphoramidon, 0.1% BSA binding buffer at pH 7.4 to a final assay concentration of 5 μg protein/well. For inhibition experiments, membranes were incubated with [³H]SR142801 at a concentration equal to K_{D} value of radioligand and 10 concentrations of the inhibitory compound (0.0003-10 μM) (in a total reaction volume of 500 μl) for 75 min at room temperature (RT). At the end of the incubation, membranes were filtered onto unitfilter (96-well white microplate with bonded GF/C filter preincubated 1 h in 0.3% PEI +0.3% BSA, Packard BioScience, Meriden, Conn.) with a Filtermate 196 harvester (Packard BioScience) and washed 4 times with ice-cold 50 mM Tris-HCl, pH 7.4 buffer. Nonspecific binding was measured in the presence of 10 μM SB222200 for both radioligands. The radioactivity on the filter was counted (5 min) on a Packard Top-count microplate scintillation counter with quenching correction after addition of 45 μl of microscint 40 (Canberra Packard S.A., Zütrich, Switzerland) and shaking for 1 h. Inhibition curves were fitted according to the Hill equation: y 32 100/(1+(x/IC₅₀)^{nH}), where n_{H}=slope factor using Excel-fit 4 software (Microsoft). IC₅₀ values were derived from the inhibition curve and the affinity constant (Kᵢ) values were calculated using the Cheng-Prussoff equation Kᵢ=IC₅₀/(1+[L]/K_{D}) where [L] is the concentration of radioligand and K_{D} is its dissociation constant at the receptor, derived from the saturation isotherm. All experiments were performed in duplicate and the mean +standard error (SEM) of the individual Kᵢ values was calculated.

Results of some representative compounds of the hNK-3 receptor affinity are shown in the following Table:

| Example No. | Kᵢ NK3 h (μM) |
|---|---|
| 45 | 0.0085 |
| 46 | 0.0024 |
| 47 | 0.0021 |
| 48 | 0.0036 |
| 49 | 0.0055 |
| 57 | 0.0059 |
| 58 | 0.0036 |
| 59 | 0.0043 |
| 60 | 0.0026 |
| 69 | 0.0024 |
| 70 | 0.0012 |
| 71 | 0.0028 |
| 72 | 0.0022 |
| 73 | 0.0015 |
| 74 | 0.0013 |
| 75 | 0.0039 |
| 76 | 0.0017 |
| 77 | 0.0031 |
| 78 | 0.0036 |
| 79 | 0.0015 |
| 81 | 0.003 |
| 82 | 0.0012 |
| 83 | 0.0023 |
| 84 | 0.0021 |
| 85 | 0.0014 |
| 86 | 0.0014 |
| 87 | 0.0046 |
| 88 | 0.0018 |
| 89 | 0.0053 |
| 90 | 0.0025 |
| 94 | 0.003 |
| 95 | 0.0047 |
| 96 | 0.0072 |
| 99 | 0.0058 |
| 100 | 0.003 |
| 101 | 0.0068 |
| 102 | 0.0051 |
| 103 | 0.0022 |
| 104 | 0.0023 |
| 105 | 0.0087 |
| 106 | 0.0051 |
| 109 | 0.0056 |
| 115 | 0.0081 |
| 119 | 0.0008 |
| 120 | 0.002 |
| 122 | 0.0015 |
| 127 | 0.0052 |
| 129 | 0.0082 |
| 131 | 0.0034 |
| 136 | 0.0096 |
| 146 | 0.0007 |
| 148 | 0.0038 |
| 150 | 0.0017 |
| 152 | 0.0011 |
| 162 | 0.0005 |
| 163 | 0.0056 |
| 164 | 0.0008 |
| 166 | 0.0022 |
| 168 | 0.003 |

The present invention also provides pharmaceutical compositions containing compounds of the invention, for example, compounds of formula I or pharmaceutically acceptable salts thereof and a pharmaceutically acceptable carrier. Such pharmaceutical compositions can be in the form of tablets, coated tablets, dragees, hard and soft gelatin capsules, solutions, emulsions or suspensions. The pharmaceutical compositions also can be in the form of suppositories or injectable solutions.

The pharmaceutical compositions of the invention, in addition to one or more compounds of the invention, contain a pharmaceutically acceptable carrier. Suitable pharmaceutically acceptable carriers include pharmaceutically inert, inorganic or organic carriers. Lactose, corn starch or derivatives thereof, talc, stearic acid or its salts etc can be used as such excipients e.g. for tablets, dragees and hard gelatin capsules. Suitable excipients for soft gelatin capsules are e.g. vegetable oils, waxes, fats, semi-solid and liquid polyols etc. Suitable excipients for the manufacture of solutions and syrups are e.g. water, polyols, saccharose, invert sugar, glucose etc. Suitable excipients for injection solutions are e.g. water, alcohols, polyols, glycerol, vegetable oils etc. Suitable excipients for suppositories are e.g. natural or hardened oils, waxes, fats, semi-liquid or liquid polyols etc.

Moreover, the pharmaceutical compositions can contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

The present invention also provides a method for the manufacture of pharmaceutical compositions. Such process comprises bringing one or more compounds of formula I and/or pharmaceutically acceptable acid addition salts thereof and, if desired, one or more other therapeutically valuable substances into a galenical administration form together with one or more therapeutically inert carriers.

The compounds and compositions of the present invention can be administered in a conventional manner, for example, orally, rectally, or parenterally. The pharmaceutical compositions of the invention can be administered orally, for example, in the form of tablets, coated tablets, dragees, hard and soft gelatin capsules, solutions, emulsions, or suspensions. The pharmaceutical compositions also can be administered rectally, for example, in the form of suppositories, or parenterally, for example, in the form of injectable solutions.

The dosage at which compounds of the invention can be administered can vary within wide limits and will, of course, be fitted to the individual requirements in each particular case. In general, in the case of oral administration a daily dosage of about 10 to 1000 mg per person of a compound of general formula I should be appropriate, although the above upper limit can also be exceeded when necessary.

### Example A

Tablets of the following composition are manufactured in the usual manner:

| | mg/tablet |
|---|---|
| Active substance | 5 |
| Lactose | 45 |
| Corn starch | 15 |
| Microcrystalline cellulose | 34 |
| Magnesium stearate | 1 |
| Tablet weight | 100 |

### Example B

Capsules of the following composition are manufactured:

| | mg/capsule |
|---|---|
| Active substance | 10 |
| Lactose | 155 |
| Corn starch | 30 |
| Talc | 5 |
| Capsule fill weight | 200 |

The active substance, lactose and corn starch can be firstly mixed in a mixer and then in a comminuting machine. The mixture can be returned to the mixer, the talc can be added thereto and mixed thoroughly. The mixture then can be filled by machine into hard gelatin capsules.

### Example C

Suppositories of the following composition are manufactured:

| | mg/supp. |
|---|---|
| Active substance | 15 |
| Suppository mass | 1285 |
| Total | 1300 |

The suppository mass can be melted in a glass or steel vessel, mixed thoroughly and cooled to 45° C. Thereupon, the finely powdered active substance can be added thereto and stirred until it has dispersed completely. The mixture can be poured into suppository moulds of suitable size and left to cool. The suppositories then can be removed from the moulds and packed individually in wax paper or metal foil.

1. A compound of formula I 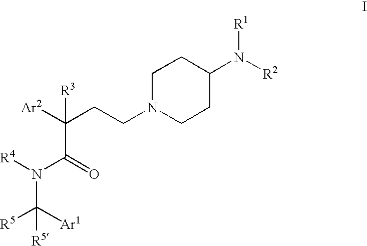
wherein
Ar¹ and Ar²are each independently phenyl or pyridinyl, each of which is optionally substituted by one or two substituents, selected from the group consisting of halogen, lower alkyl, lower alkoxy, lower alkyl substituted by halogen, lower alkoxy substituted by halogen, lower alkyl substituted by alkoxy, lower alkyl substituted by cyano, lower di-alkyl amino, pyridinyl and cyano;
R¹ is hydrogen, lower alkyl, —(CH₂)₂O-lower alkyl, or cycloalkyl:
R² is —S(O)₂-lower alkyl or —C(O)-lower alkyl;
or R¹ and R² together with the N-atom to which they are attached form a pyrrolidin-2-one or a piperidin-2-one group;
R³ is hydrogen, halogen or lower alkyl;
R⁴ is hydrogen or lower alkyl;
R⁵ and R^{5′} are each independently hydrogen, lower alkyl, lower alkyl substituted by halogen, lower alkyl substituted by hydroxy or cycloalkyl;
or R⁵ and R^{5′} together with the carbon-atom to which they are attached form a cycloalkyl group;
or a pharmaceutically active salt thereof.

2. The compound of claim 1, wherein Ar¹ and Ar² are both phenyl.

3. The compound of claim 2, wherein R¹ is methyl and R² is S(O)₂CH₃.

4. The compound of claim 3, selected from the group consisting of
2-(3,4-dichloro-phenyl)-N-(3-fluoro-4-trifluoromethyl-benzyl)-4-[4-(methanesulfonyl-methyl-amino)-piperidin-1-yl]-2,N-dimethyl-butyramide,
N-(4-chloro-3-trifluoromethyl-benzyl)-2-(3,4-dichloro-phenyl)-4-[4-(methanesulfonyl-methyl-amino)-piperidin-1-yl]-2,N-dimethyl-butyramide,
2-(3,4-dichloro-phenyl)-N-(4-fluoro-3-trifluoromethyl-benzyl)-4-[4-(methanesulfonyl-methyl-amino)-piperidin-1-yl]-2,N-dimethyl-butyramide,
N-(3,4-dichloro-benzyl)-2-(3,4-dichloro-phenyl)-4-[4-(methanesulfonyl-methyl-amino)-piperidin-1-yl]-2,N-dimethyl-butyramide, and
2-(3,4-dichloro-phenyl)-N-[1-(3,4-dichloro-phenyl)-2-hydroxy-ethyl]-4-[4-(methanesulfonyl-methyl-amino)-piperidin-1-yl]-2-methyl-butyramide; diastereoisomer 1.

5. The compound of claim 2, wherein R¹ is cyclopropyl and R² is S(O)₂CH₃.

6. The compound of claim 5, selected from the group consisting of
4-[4-(cyclopropyl-methanesulfonyl-amino)-piperidin-1-yl]-2-(3,4-dichloro-phenyl)-N-(3-fluoro-4-trifluoromethyl-benzyl)-2,N-dimethyl-butyramide,
N-(4-chloro-3-trifluoromethyl-benzyl)-4-[4-(cyclopropyl-methanesulfonyl-amino)-piperidin-1-yl]-2-(3,4-dichloro-phenyl)-2,N-dimethyl-butyramide,
4-[4-(cyclopropyl-methanesulfonyl-amino)-piperidin-1-yl]-2-(3,4-dichloro-phenyl)-N-(4-fluoro-3-trifluoromethyl-benzyl)-2,N-dimethyl-butyramide, and
4-[4-(cyclopropyl-methanesulfonyl-amino)-piperidin-1-yl]-N-(3,4-dichloro-benzyl)-2-(3,4-dichloro-phenyl)-2,N-dimethyl-butyramide.

7. The compound of claim 2, wherein R¹ is ethyl and R² is S(O)₂CH₃.

8. The compound of claim 7, selected from the group consisting of
2-(3,4-dichloro-phenyl)-4-[4-(ethyl-methanesulfonyl-amino)-piperidin-1-yl]-N-(3-fluoro-4-trifluoromethyl-benzyl)-2,N-dimethyl-butyramide,
N-(3,4-dichloro-benzyl)-2-(3,4-dichloro-phenyl)-4-[4-(ethyl-methanesulfonyl-amino)-piperidin-1-yl]-2,N-dimethyl-butyramide,
N-(3,4-dichloro-benzyl)-2-(3,4-dichloro-phenyl)-4-[4-(ethyl-methanesulfonyl-amino)-piperidin-1-yl]-2-methyl-butyramide,
N-(4-chloro-benzyl)-2-(3,4-dichloro-phenyl)-4-[4-(ethyl-methanesulfonyl-amino)-piperidin-1-yl]-2,N-dimethyl-butyramide,
N-(4-chloro-3-trifluoromethyl-benzyl)-2-(3,4-dichloro-phenyl)-4-[4-(ethyl-methanesulfonyl-amino)-piperidin-1-yl]-2,N-dimethyl-butyramide,
2-(3,4-dichloro-phenyl)-4-[4-(ethyl-methanesulfonyl-amino)-piperidin-1-yl]-N-(4-fluoro-3-trifluoromethyl-benzyl)-2,N-dimethyl-butyramide,
N-(3-chloro-benzyl)-2-(3,4-dichloro-phenyl)-4-[4-(ethyl-methanesulfonyl-amino)-piperidin-1-yl]-2,N-dimethyl-butyramide,
2-(3,4-dichloro-phenyl)-4-[4-(ethyl-methanesulfonyl-amino)-piperidin-1-yl]-2,N-dimethyl-N-(4-trifluoromethyl-benzyl)-butyramide,
2-(3,4-dichloro-phenyl)-4-[4-(ethyl-methanesulfonyl-amino)-piperidin-1-yl]-2,N-dimethyl-N-(4-methyl-benzyl)-butyramide,
N-(2-chloro-benzyl)-2-(3,4-dichloro-phenyl)-4-[4-(ethyl-methanesulfonyl-amino)-piperidin-1-yl]-2,N-dimethyl-butyramide, and
2-(3,4-dichloro-phenyl)-N-[1-(3,4-dichloro-phenyl)-2-hydroxy-ethyl]-4-[4-(ethyl-methanesulfonyl-amino)-piperidin-1-yl]-2-methyl-butyramide; diastereoisomer 1.

9. The compound of claim 2, wherein R¹ and R² together with the N-atom to which they are attached form a pyrrolidin-2-one group.

10. The compound of claim 9, selected from the group consisting of
2-(3,4-dichloro-phenyl)-N-(3-fluoro-4-trifluoromethyl-benzyl)-2,N-dimethyl-4-[4-(2-oxo-pyrrolidin-1-yl)-piperidin-1-yl]-butyramide,
N-(3,4-dichloro-benzyl)-2-(3,4-dichloro-phenyl)-2,N-dimethyl-4-[4-(2-oxo-pyrrolidin-1-yl)-piperidin-1-yl]-butyramide,
N-(3,4-dichloro-benzyl)-2-(3,4-dichloro-phenyl)-2-methyl-4-[4-(2-oxo-pyrrolidin-1-yl)-piperidin-1-yl]-butyramide,
N-(4-chloro-benzyl)-2-(3,4-dichloro-phenyl)-2,N-dimethyl-4-[4-(2-oxo-pyrrolidin-1-yl)-piperidin-1-yl]-butyramide,
N-(4-chloro-3-trifluoromethyl-benzyl)-2-(3,4-dichloro-phenyl)-2,N-dimethyl-4-[4-(2-oxo-pyrrolidin-1-yl)-piperidin-1-yl]-butyramide,
2-(3,4-dichloro-phenyl)-N-(4-fluoro-3-trifluoromethyl-benzyl)-2,N-dimethyl-4-[4-(2-oxo-pyrrolidin-1-yl)-piperidin-1-yl]-butyramide,
N-(3-chloro-benzyl)-2-(3,4-dichloro-phenyl)-2,N-dimethyl-4-[4-(2-oxo-pyrrolidin-1-yl)-piperidin-1-yl]-butyramide,
2-(3,4-dichloro-phenyl)-2,N-dimethyl-4-[4-(2-oxo-pyrrolidin-1-yl)-piperidin-1-yl]-N-(4-trifluoromethyl-benzyl)-butyramide, and
2-(3,4-dichloro-phenyl)-2,N-dimethyl-N-(4-methyl-benzyl)-4-[4-(2-oxo-pyrrolidin-1-yl)-piperidin-1-yl]-butyramide.

11. The compound of claim 9, selected from the group consisting of
N-(2-chloro-benzyl)-2-(3,4-dichloro-phenyl)-2,N-dimethyl-4-[4-(2-oxo-pyrrolidin-1-yl)-piperidin-1-yl]-butyramide,
2-(3,4-dichloro-phenyl)-N-[-1-(3,4-dichloro-phenyl)-2-hydroxy-ethyl]-2-methyl-4-[4-(2-oxo-pyrrolidin-1-yl)-piperidin-1-yl]-butyramide; diastereoisomer 1,
2-(3,4-dichloro-phenyl)-2-methyl-4-[4-(2-oxo-pyrrolidin-1-yl)-piperidin-1-yl]-N-(2,2,2-trifluoro-1-phenyl-ethyl)-butyramide,
2-(3,4-dichloro-phenyl)-2-methyl-4-[4-(2-oxo-pyrrolidin-1-yl)-piperidin-1-yl]-N-[2,2,2-trifluoro-1-(4-fluoro-phenyl)-ethyl]-butyramide,
N-(4-chloro-benzyl)-2-(3,4-dichloro-phenyl)-N-methyl-4-[4-(2-oxo-pyrrolidin-1-yl)-piperidin-1-yl]-butyramide,
(R or S)-2-(3,4-dichloro-phenyl)-N-(4-fluoro-3-trifluoromethyl-benzyl)-2,N-dimethyl-4-[4-(2-oxo-pyrrolidin-1-yl)-piperidin-1-yl]-butyramide,
(R or S)-2-(3,4-dichloro-phenyl)-N-[(S or R)-1-(3,4-dichloro-phenyl)-2-hydroxy-ethyl]-2-methyl-4-[4-(2-oxo-pyrrolidin-1-yl)-piperidin-1-yl]-butyramide,
2-(3,4-dichloro-phenyl)-2-methyl-4-[4-(2-oxo-pyrrolidin-1-yl)-piperidin-1-yl]-N-((S)-1-phenyl-propyl)-butyramide,
2-(3,4-dichloro-phenyl)-N—[(R)-1-(4-fluoro-phenyl)-2-hydroxy-ethyl]-2-(R or S) methyl-4-[4-(2-oxo-pyrrolidin-1-yl)-piperidin-1-yl]-butyramide, and
2-(3,4-dichloro-phenyl)-N-(4-fluoro-3-trifluoromethyl-benzyl)-2-methyl-4-[4-(2-oxo-pyrrolidin-1-yl)-piperidin-1-yl]-butyramide.

12. The compound of claim 2, wherein R¹ is lower alkyl and R² is C(O)CH₃.

13. The compound of claim 11, selected from the group consisting of
4-[4-(acetyl-methyl-amino)-piperidin-1-yl]-2-(3,4-dichloro-phenyl)-N-(3-fluoro-4-trifluoromethyl-benzyl)-2-methyl-butyramide,
4-[4-(acetyl-methyl-amino)-piperidin-1-yl]-N-(3,4-dichloro-benzyl)-2-(3,4-dichloro-phenyl)-2,N-dimethyl-butyramide,
4-[4-(acetyl-methyl-amino)-piperidin-1-yl]-N-(3,4-dichloro-benzyl)-2-(3,4-dichloro-phenyl)-2-methyl-butyramide,
4-[4-(acetyl-methyl-amino)-piperidin-1-yl]-N-(4-chloro-benzyl)-2-(3,4-dichloro-phenyl)-2,N-dimethyl-butyramide,
4-[4-(acetyl-methyl-amino)-piperidin-1-yl]-N-(4-chloro-3-trifluoromethyl-benzyl)-2-(3,4-dichloro-phenyl)-2,N-dimethyl-butyramide,
4-[4-(acetyl-methyl-amino)-piperidin-1-yl]-2-(3,4-dichloro-phenyl)-N-(4-fluoro-3-trifluoromethyl-benzyl)-2,N-dimethyl-butyramide,
4-[4-(acetyl-methyl-amino)-piperidin-1-yl]-N-(3-chloro-benzyl)-2-(3,4-dichloro-phenyl)-2,N-dimethyl-butyramide, and
4-[4-(acetyl-methyl-amino)-piperidin-1-yl]-2-(3,4-dichloro-phenyl)-2,N-dimethyl-N-(4-trifluoromethyl-benzyl)-butyramide.

14. The compound of claim 11, selected from the group consisting of
4-[4-(acetyl-methyl-amino)-piperidin-1-yl]-2-(3,4-dichloro-phenyl)-N-[1-(3,4-dichloro-phenyl)-2-hydroxy-ethyl]-2-methyl-butyramide; diastereoisomer 1,
(R or S)-4-[4-(acetyl-methyl-amino)-piperidin-1-yl]-2-(3,4-dichloro-phenyl)-N-(4-fluoro-3-trifluoromethyl-benzyl)-2,N-dimethyl-butyramide,
4-[4-(acetyl-methyl-amino)-piperidin-1-yl]-N-[-1-(4-chloro-phenyl)-2-hydroxy-ethyl]-2-(3,4-dichloro-phenyl)-2-methyl-butyramide (diastereosisomer 1),
4-[4-(acetyl-propyl-amino)-piperidin-1-yl]-2-(3,4-dichloro-phenyl)-N-[(R)-1-(4-fluoro-phenyl)-2-hydroxy-ethyl]-2-methyl-butyramide (epimer 1),
4-[4-(acetyl-propyl-amino)-piperidin-1-yl]-2-(3,4-dichloro-phenyl)-N-[(R)-1-(4-fluoro-phenyl)-2-hydroxy-ethyl]-2-methyl-butyramide (epimer 2),
4-[4-(acetyl-propyl-amino)-piperidin-1-yl]-2-(3,4-dichloro-phenyl)-2-methyl-N-((S)-1-phenyl-propyl)-butyramide,
4-[4-(acetyl-ethyl-amino)-piperidin-1-yl]-2-(3,4-dichloro-phenyl)-N-[(R)-1-(4-fluoro-phenyl)-2-hydroxy-ethyl]-2-methyl-butyramide (epimer 1), and
4-[4-(acetyl-ethyl-amino)-piperidin-1-yl]-2-(3,4-dichloro-phenyl)-2-methyl-N-((S)-1-phenyl-propyl)-butyramide.

15. The compound of claim 2, wherein R¹ is —(CH₂)₂OCH₃ and R² is C(O)CH₃.

16. The compound of claim 13, selected form the group consisting of
4-{4-[acetyl-(2-methoxy-ethyl)-amino]-piperidin-1-yl}-2-(3,4-dichloro-phenyl)-N-[1-(4-fluoro-phenyl)-2-hydroxy-ethyl]-2-methyl-butyramide (diastereoisomer 1),
4-{4-[acetyl-(2-methoxy-ethyl)-amino]-piperidin-1-yl}-2-(3,4-dichloro-phenyl)-N-[-1-(3,4-dichloro-phenyl)-2-hydroxy-ethyl]-2-methyl-butyramide (diastreoisomer 1),
4-{4-[acetyl-(2-methoxy-ethyl)-amino]-piperidin-1-yl}-2-(3,4-dichloro-phenyl)-N-[(R)-1-(4-fluoro-phenyl)-2-hydroxy-ethyl]-2-(R or S) methyl-butyramide, and
4-{4-[acetyl-(2-methoxy-ethyl)-amino]-piperidin-1-yl}-2-(3,4-dichloro-phenyl)-2-methyl-N—((S)-1-phenyl-propyl)-butyramide.

17. A pharmaceutical composition comprising a therapeutically effective amount of a compounds of formula I 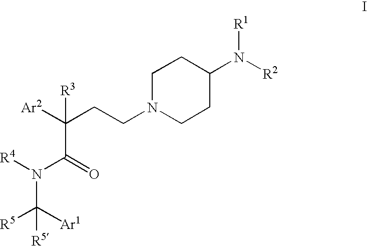
wherein
Ar¹ and Ar²are each independently phenyl or pyridinyl, each of which is optionally substituted by one or two substituents, selected from the group consisting of halogen, lower alkyl, lower alkoxy, lower alkyl substituted by halogen, lower alkoxy substituted by halogen, lower alkyl substituted by alkoxy, lower alkyl substituted by cyano, lower di-alkyl amino, pyridinyl and cyano;
R¹ is hydrogen, lower alkyl, —(CH₂)₂O-lower alkyl, or cycloalkyl:
R² is 13 S(O)₂-lower alkyl or —C(O)-lower alkyl;
or R¹ and R² together with the N-atom to which they are attached form a pyrrolidin-2-one or a piperidin-2-one group;
R³ is hydrogen, halogen or lower alkyl;
R⁴ is hydrogen or lower alkyl;
R⁵ and R^{5′} are each independently hydrogen, lower alkyl, lower alkyl substituted by halogen, lower alkyl substituted by hydroxy or cycloalkyl;
or R⁵ and R^{5′} together with the carbon-atom to which they are attached form a cycloalkyl group;
or a pharmaceutically active salt thereof and a pharmaceutically acceptable carrier.
